(19) United States
(12) Patent Application Publication
(10) Pub. No.: US 2016/0058829 A1
(43) Pub. Date: Mar. 3, 2016

(54) ANTI-FIBROTIC PEPTIDES AND THEIR USE IN METHODS FOR TREATING DISEASES AND DISORDERS CHARACTERIZED BY FIBROSIS

(71) Applicant: Thrasos Innovation, Inc., Montreal (CA)

(72) Inventors: Dattatreyamurty BOSUKONDA, Shrewsbury, MA (US); Peter C. KECK, Millbury, MA (US)

(21) Appl. No.: 14/818,652

(22) Filed: Aug. 5, 2015

Related U.S. Application Data
(63) Continuation of application No. 13/553,685, filed on Jul. 19, 2012, now abandoned.
(60) Provisional application No. 61/509,340, filed on Jul. 19, 2011.
(60) Provisional application No. 61/662,337, filed on Jun. 20, 2012.

(51) Int. Cl. A61K 38/10

(52) U.S. Cl. CPC A61K 38/10

(57) ABSTRACT

The invention provides methods and compositions for inhibiting and/or reversing fibrosis. The invention further provides peptides and polypeptides which are BMP agonists which trigger BMP signaling and inhibit and/or reverse EMT in a cell or tissue.

### PRIORITY AND INCORPORATION BY REFERENCE

This application is a continuation application of U.S. patent application Ser. No. 13/553,685, filed Jul. 19, 2012, which claims priority to U.S. Provisional Application No. 61/662,337, filed Jun. 20, 2012, and U.S. Provisional Application No. 61/509,340, filed Jun. 19, 2011. The entire contents of each of the aforementioned applications are incorporated herein by reference in their entirety. All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND OF THE INVENTION

1. Field of the Invention

The present invention relates to compositions of matter, methods of manufacture of same, and methods for treating fibrosis and/or conditions relating to fibrosis. The invention further relates to design, preparation, and use of polypeptides or peptides for treating fibrosis and/or underlying conditions which result in fibrosis, including reversal and/or inhibition of the epithelial-mesenchymal transition (EMT) process.

2. Background

Fibrosis occurs when the body's natural healing processes go awry, being generally characterized by excessive overgrowth, hardening, and/or scarring of a tissue in response to a chronic inflammatory condition associated with some type of underlying cause, such as tissue damage, infection, autoimmune reactions, chemical insults, allergic responses, toxins, radiation, mechanical injury, or other various persistent stimuli (T A Wynn, J. Pathol., 2008, 214: 199-210). While the range of clinical and etiological manifestations may be wide, fibrotic disorders are similar in that they generally share some kind of underlying persistent irritant that continues to promote the release of various growth factors, proteolytic enzymes, angiogenic factors, and fibrogenic cytokines which lead to increased and excessive accumulation of extracellular matrix components that progressively damage normal tissue and change its cellular architecture to the point functionality is lost. This process usually occurs over many months and years and can eventually lead to organ dysfunction or death. Examples of common fibrotic diseases include, for example, diabetic nephropathy, liver cirrhosis, idiopathic pulmonary fibrosis, rheumatoid arthritis, atherosclerosis, cardiac fibrosis, systemic sclerosis, nepthritis, and scleroderma (Id.)

Typically, the natural healing process following tissue damage begins with the release of inflammatory mediators by epithelial and/or endothelial cells local to the site of damage which initiates a healing cascade beginning with platelet-induced blood clot formation and the formation of a provisional extracellular matrix (ECM). Platelet degranulation also leads to vasodilation and increased permeability of blood vessels, while activated myofibroblasts and epithelial and/or endothelial cells produce matrix metalloproteinases (MMPs) which further disrupt the basement membrane allowing greater recruitability of further inflammatory cells to the injury site (Id.). The cells also produce various growth factors, cytokines, and chemokines which stimulate the recruitment and proliferation of additional immune system cells to the site, leading to a cascade that results, among other responses, in angiogenesis and the secretion of profibrotic cytokines and growth factors by activated lymphocytes, including Tumor Growth Factor-β (TGF-β). These events further activate fibroblasts, which become transformed into myofibroblasts, which migrate into the wound and facilitate wound contraction. At the site of the contracting wound, the epithelial and/or endothelial cells divide to regenerate the damaged tissue thereby completing the natural wound healing process (Id).

Fibrosis differs from this process because of the presence of a persistent and chronic inflammation condition, which triggers a cascade that includes the excessive accumulation of ECM materials which are not turned over and which ultimately leads to the generation or formation of scar tissue, and concomitant organ or tissue dysfunction caused by the scarring (Id).

Pro-fibrotic proteins such as transforming growth factor-beta (TGF-β) and connective tissue growth factor (CTGF) have been implicated to be involved in fibrotic diseases. As TGF-β induces fibroblasts to synthesize ECM, this cytokine has long been believed to be a central mediator of the fibrotic response (LeRoy et al., Eur. Cytokine Netw., 1:215-219). CTGF, discovered more than a decade ago as a protein secreted by human endothelial cells (Bradman et al., J. Cell Biol., 1991, 114: 1285-1294), is induced by TGF-β and is considered a downstream mediator of the effects of TGF-β on fibroblasts (Leask et al., J. Invest. Dermatol., 2004, 122:1-6; Grotendorst, G. R., Cytokine GrowthFactor Rev., 1997, 8:171-179). Similarly, TGF-β induces expression of the ED-A form of the matrix protein fibronectin (ED-A FN), a variant of fibronectin that occurs through alternative splicing of the fibronectin transcript (Oyama et al., Biochemistry, 1989, 28:1428-1434). This induction of ED-A FN is required for TGF-β1-triggered enhancement of α-SMA and collagen type I expression (Serini et al., J. Cell Biol., 142:873-881). Thus TGF-β has been implicated as a “master switch” in induction of fibrosis in many tissues, including, for example, lung (Sime et al., Clin. Immunol., 2001, 99:308-319) and kidney (Lan, Int. J. Biol. Sci., 2011, 7:1056-1067). In this regard, TGF-β is upregulated in lungs of patients with idiopathic pulmonary fibrosis, or in kidneys of chronic kidney disease patients and expression of active TGF-β in lungs or kidneys of rats induces a dramatic fibrotic response, whereas the inability to respond to TGF-β1 affords protection from bleomycin-induced fibrosis (Zhao et al., Am. J. Physiol. Lung Cell Mol. Physiol., 2002, 282: L585-L593) or renal interstitial fibrosis (Zeisberg et al., Nat Med, 2003, 9: 964-8).

The process of epithelial-mesenchymal transition (EMT) has also been widely implicated as a common mechanism by which damaged tissues undergo the fibrotic response. EMT is a process whereby fully differentiated epithelial cells undergo transition to a mesenchymal phenotype, which then gives rise to fibroblasts and myofibroblasts, and is increasingly being recognized as playing a central role in fibrosis and scar formation following epithelial injury. The extent to which this process contributes to fibrosis following injury in the lung and other organs is a subject of active investigation. Recently, it was demonstrated that transforming growth factor (TGF)-β induces EMT in alveolar epithelial cells (AEC) in vitro and in vivo, and epithelial and mesenchymal markers have been colocalized to hyperplastic type II (AT2) cells in lung tissue from patients with idiopathic pulmonary fibrosis (IPF), suggesting that AEC may exhibit extreme plasticity and serve as a source of fibroblasts and/or myofibroblasts in lung fibrosis. TGF-β1 was first described as an inducer of EMT in normal mammary epithelial cells (Miettinen et al., 1994, 127: 2021-2036) and has since been shown to mediate EMT in vitro in a number of different epithelial cells, including renal proximal tubular, lens, and most recently alveolar epithelial cells (Fan et al., Kidney Int, 1999, 56: 1455-1467; Hales et al., Curr Eye Res, 1994, 13:885-890; Kasai et al., Respir Res, 2005, 6:56; Saika et al., Am J Pathol, 2004, 164:651-663; and Willis et al., Am J Pathol, 2005, 166:1321-1332). Accordingly, EMT may play a common, universal role in fibrosis, no matter the underlying disease etiology.

Despite the extent of knowledge (or lack thereof) regarding fibrosis and its underlying molecular processes, fibrotic disease represents one of the largest groups of disorders for which there is no effective therapy and thus represents a major unmet medical need. Often the only redress for patients with fibrosis is organ transplantation. However, since the supply of organs is insufficient to meet the demand, patients often die while waiting to receive suitable organs. Lung fibrosis alone can be a major cause of death in scleroderma lung disease, idiopathic pulmonary fibrosis, radiation- and chemotherapy-induced lung fibrosis and in conditions caused by occupational inhalation of dust particles. The lack of appropriate anti-fibrotic therapies arises primarily because the etiology of fibrotic disease is essentially unknown. It will be critical to understand how normal tissue repair is controlled and how this process goes awry in fibrotic disease in order to identify effective therapeutic approaches.

New therapeutic solutions that are capable of treating fibrotic conditions would advance the art. In particular, therapies that successfully target underlying causes common to any type of fibrotic disease and which are capable of slowing, reversing, and/or eliminating fibrosis or the underlying molecular processes, including EMT, causing fibrosis.

### SUMMARY OF THE INVENTION

The present invention is based, in part, on the discovery by the inventors that a subclass of previously-disclosed polypeptides/peptides are agonists of BMP (bone morphogenetic protein) receptors, including both Type I and Type II receptors, and that such polypeptides/peptides are capable of inhibiting and/or reversing epithelial to mesenchymal transition (EMT) and fibrosis and can thus be used to therapeutically treat fibrosis and conditions relating to or involving fibrosis. Accordingly, the present invention relates to the design, preparation, and use of certain polypeptides/peptides for treating, inhibiting, reversing, and/or eliminating fibrosis and/or certain underlying conditions which result or cause a fibrotic condition, including EMT. The utility of the present invention, and in particular, to the polypeptides/peptides and methods of the invention, extend to the treatment of any fibrotic condition in any tissue and/or organ of the body, including, but not limited to, fibrosis associated with diabetic nephropathy, liver cirrhosis, idiopathic pulmonary fibrosis, rheumatoid arthritis, atherosclerosis, cardiac fibrosis, systemic sclerosis, nepthritis, and scleroderma

Recently, it was found that transforming growth factor β (TGF-β), as a central mediator of fibrogenesis, is an inducer of EMT, which in turn, mediates fibrosis. It was further identified that BMP-7 reversed TGF-β-induced EMT, thereby suggesting the role of BMP-7 in counteracting fibrosis occurring via EMT. The present inventors discovered that a particular subclass (as further described herein) of previously disclosed peptides are BMP agonists, i.e., peptides which mimic BMP or a specific subportion thereof and which bind and activate BMP signaling via BMP receptors, were effective in the inhibition and/or reversal of EMT and fibrosis relating to a variety of conditions, including diabetic nephropathy, liver cirrhosis, idiopathic pulmonary fibrosis, rheumatoid arthritis, atherosclerosis, cardiac fibrosis, systemic sclerosis, nepthritis, and scleroderma.

Accordingly, in a first aspect, the present invention relates to certain peptides or polypeptides (i.e., interchangeably which may be referred to as “compounds,” “peptides,” or “polypeptides” of the invention) which are agonists of a BMP receptor, including type I and type II receptors, and which are a subclass of previously disclosed peptides. It has been discovered that the BMP-agonist compounds of the invention induce BMP signaling, thereby mimicking BMP's counteractive effect on TGF-β-induced EMT and fibrosis. In other aspects, the present invention provides methods for making the BMP-agonist peptides of the invention, including via biological and chemical or synthetic processes. In still other aspects, the present invention relates to isolated nucleic acid molecules which encode the peptides of the invention, or propeptides (which may be cleaved or otherwise modified to form a desired BMP-agonist peptide of the invention), which include nucleic acid molecules used for making the peptides of the invention in vitro or in vivo, e.g., as in for purposes of somatic gene transfer as a means to deliver the peptides of the invention to a subject in need thereof. In yet other aspects, the present invention relates to pharmaceutical compositions of matter which include one or more peptides of the invention, or propeptides of the invention, or one or more nucleic acid molecules encoding such peptides or propeptides, and one or more pharmaceutically acceptable carriers. In still another aspect, the present invention relates to methods for administering therapeutically effective amounts of the peptides or pharmaceutical compositions of the invention to treat or prevent (i.e., prophylactic administration) fibrosis or a related underlying condition that results in fibrosis (e.g., EMT) in a subject having a fibrotic disease, including, but not limited to the treatment of diabetic nephropathy, liver cirrhosis, idiopathic pulmonary fibrosis, rheumatoid arthritis, atherosclerosis, cardiac fibrosis, systemic sclerosis, nepthritis, and scleroderma. In yet another aspect, the present invention relates to kits or pharmaceutical packages which have one or more containers, one or more of the peptides or polypeptides of the invention or a pharmaceutical composition comprising same, and instructions for use the contents of the kit or pharmaceutical package.

In particular embodiments, the BMP-agonist peptides of the invention can include peptides having amino acid sequences selected from the group consisting of SEQ ID NOs: 1-77 (as shown in Table 1 or otherwise herein below). In certain other embodiments, the BMP-agonist peptides of the invention can include peptides that have a similar sequence to those peptides of SEQ ID NOs: 1-77, and which specifically may include peptides have an amino acid sequence that has at least 99% or greater sequence identity to any of SEQ ID NOs: 1-77, or at least 95% or greater sequence identity to any of SEQ ID NOs: 1-77, or at least 90% or greater sequence identity to any of SEQ ID NOs: 1-77, or at least 85% or greater sequence identity to any of SEQ ID NOs: 1-77, or at least 80% or greater sequence identity to any of SEQ ID NOs: 1-77, or at least 75% or greater sequence identity to any of SEQ ID NOs: 1-77, or at least 70% or greater sequence identity to any of SEQ ID NOs: 1-77, or at least 65% or greater sequence identity to any of SEQ ID NOs: 1-77, or at least 60% or greater sequence identity to any of SEQ ID NOs: 1-77.

In other embodiments, the BMP-agonist peptides of the invention can include any suitable variants, analogs, homologs, or fragments of the peptides of the invention (and/or propeptides as the case may be), and small molecules related to these. In one embodiment, the peptides modulate the epithelial to mesenchymal transition (EMT) process. In another embodiment, the peptides modulate fibrosis. In particular embodiments, the BMP-agonist peptides of the invention, which may include any suitable variant, analog, homolog, or fragment thereof, mimic the BMP signaling process. In other particular embodiments, the BMP-agonist peptides of the invention, which may include any suitable variant, analog, homolog, or fragment thereof, will counteract, inhibit, and/or reverse TGF-β-induced EMT. In yet other embodiments, the BMP-agonist peptides of the invention, which may include any suitable variant, analog, homolog, or fragment thereof, will inhibit, reverse, or otherwise eradicate fibrosis.

In another embodiment, the isolated nucleic acid molecules of the invention comprise a nucleotide sequence that encodes those peptides of SEQ ID NOs: 1-77 of Table 1 or any peptide or propeptide in the scope of the invention other than those particular embodiments of Table 1. In yet another embodiment, the isolated nucleic acid molecules can be a DNA expression or cloning vector, and the vector may optionally include a promoter sequence that can be operably linked to the nucleic acid, where the promoter causes expression of the nucleotide sequence encoding the peptides or propeptides of the invention. In still another embodiment, the vector can be transformed into a cell, such as a prokaryotic or eukaryotic cell, preferably a mammalian cell, or more preferably a human cell. In even another embodiment, the vector can be a viral vector capable of infecting a mammalian cell and causing expression of a polypeptide of SEQ ID NOs: 1-77 in an animal infected with the virus. In still other embodiments, the nucleic acid molecule comprises any suitable and/or advantageous elements for executing effective expression in a host cell, whether said host cell is a prokaryotic or eukaryotic host cell and whether the expression is carried out in vitro or in vivo. In yet further embodiments, the nucleic acid molecule of the invention may comprise a somatic gene transfer vector for introducing a nucleic acid sequence that encodes a peptide of the invention, or any variant, analog, homolog, or fragment thereof, including any useful propeptide thereof, for administering to a subject in need thereof a peptide of the invention by somatic gene transfer.

In the case of propeptides, said propeptides are inactive forms of the peptides of the invention, which may be activated under certain conditions. Methods for making prodrugs or proantibodies are known. In one embodiment, the propeptides may include one or more additional polypeptide sequences that are joined to a peptide of interest. In one form, the propeptide is single polypeptide translational product that includes a leader or terminal portion of the complete polypeptide sequence that is initially present with the expression of the product and which reduces or eliminates or masks the activity of the peptide of interest. The leader or terminal portion, once removed (e.g., by protease cleavage) cause the peptide to regain its BMP signaling activity.

In embodiments of the pharmaceutical compositions of the invention, the compositions can include a peptide or polypeptide of the invention with or without a pharmaceutically acceptable carrier.

In other embodiments of the pharmaceutical compositions of the invention, the compositions of the invention can include one or more additional active agents. The one or more additional active agents can include other anti-fibrosis therapies. The one or more additional active agents can also include other therapies relating to the underlying disease or condition that results in or is involved in or relates to the fibrotic condition. For example, in certain embodiments where the fibrosis is a component of diabetic nephropathy, liver cirrhosis, idiopathic pulmonary fibrosis, rheumatoid arthritis, atherosclerosis, cardiac fibrosis, systemic sclerosis, nepthritis, and scleroderma, the additional one or more active agents can include an agent that is effective against treating other symptoms or aspects of these underlying conditions that are different from the fibrosis itself.

In aspects involving the making and/or preparation of the peptides of the invention, the invention relates to certain embodiments that involve the method of culturing a cell containing a nucleic acid molecule encoding SEQ ID NOs: 1-77 under conditions that provide for expression of the peptide; and recovering the expressed peptide. In certain other embodiments, the nucleic acid molecules can encode a suitable variant, analog, homolog, or fragment of SEQ ID NOs: 1-77, or of any other BMP-agonist peptide of the invention.

In aspects involving a kit, in certain embodiments, the kit of the invention includes one or more containers, a peptide or pharmaceutical composition described herein and instructions for using the contents therein. The peptide, in certain embodiments, may be a suitable variant, analog, homolog, or fragment of SEQ ID NOs: 1-77, or of any other BMP-agonist peptide of the invention. The kit in other embodiments may include one or more other active agents, such as those that may be active for treating a condition that results in or includes a fibrotic element, e.g., a second agent for treating diabetic nephropathy, liver cirrhosis, idiopathic pulmonary fibrosis, rheumatoid arthritis, atherosclerosis, cardiac fibrosis, systemic sclerosis, nepthritis, or scleroderma. The kit, in still other embodiments, may also include an isolated nucleic acid molecule which encodes a BMP-agonist peptide of the invention, or a variant, analog, homolog, or fragment of SEQ ID NOs: 1-77, or of any other BMP-agonist peptide of the invention. The nucleic acid molecule of the kit may be suitable for somatic gene transfer in a method of treatment of fibrosis in a subject in need thereof.

In aspects that involve the use of the peptides of the invention, or nucleic acid molecules that encode peptides of the invention, for treating fibrosis or for treating a condition that relates to or underlies the fibrotic condition, the invention provides, in various embodiments, that the fibrosis under treatment relates to diabetic nephropathy, liver cirrhosis, idiopathic pulmonary fibrosis, rheumatoid arthritis, atherosclerosis, cardiac fibrosis, systemic sclerosis, nepthritis, or scleroderma. In certain embodiments, the invention provides a method for treating fibrosis associated with chronic kidney disease (CKD), i.e., renal fibrosis associated with CKD. In certain other embodiments, the method of the invention involves treating idiopathic pulmonary fibrosis. In still other embodiments, the present invention relates to a method for treating fibrosis associated with liver cirrhosis. In yet other embodiments, the invention provides a method for treating cardiac fibrosis. In other embodiments, the invention provides a method for treating fibrosis associated with atherosclerosis. In yet other embodiments, the invention provides a method for treating fibrosis associated with scleroderma.

In certain embodiments, the invention provides methods for treating, inhibiting, and/or reversing fibrosis associated with a disease process, e.g., fibrosis associated with diabetic nephropathy, liver cirrhosis, idiopathic pulmonary fibrosis, rheumatoid arthritis, atherosclerosis, cardiac fibrosis, systemic sclerosis, nepthritis, or scleroderma, by administering to the subject a therapeutically effective amount of a BMP-agonist peptide of the invention, or a variant, analog, homolog, or fragment thereof, including any one or more of those peptides identified as SEQ ID NOs: 1-77 in Table 1 via a suitable means. Administration of the peptides of the invention may be by a suitable means, including orally, parenterally, infusion, injection, inhalation, or via the skin or through any viable means. In still another embodiment, the peptides of the invention (including any propeptides, or any variants, analogs, homologs, or fragments of the peptides of the invention) can be delivered as nucleic acid molecule which are designed to encode and express said peptides of the invention in a host in vivo.

In the methods of the invention, the administered BMP-agonist peptides of the invention can include peptides that have a similar sequence to those peptides of SEQ ID NOs: 1-77, and which specifically may include peptides having an amino acid sequence that has at least 99% or greater sequence identity to any of SEQ ID NOs: 1-77, or at least 95% or greater sequence identity to any of SEQ ID NOs: 1-77, or at least 90% or greater sequence identity to any of SEQ ID NOs: 1-77, or at least 85% or greater sequence identity to any of SEQ ID NOs: 1-77, or at least 80% or greater sequence identity to any of SEQ ID NOs: 1-77, or at least 75% or greater sequence identity to any of SEQ ID NOs: 1-77, or at least 70% or greater sequence identity to any of SEQ ID NOs: 1-77, or at least 65% or greater sequence identity to any of SEQ ID NOs: 1-77, or at least 60% or greater sequence identity to any of SEQ ID NOs: 1-77.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts a quantitative colorimetric analysis of E-cadherin fluorescence for two concentrations (100 μM and 200 μM) of the identified peptides, SEQ ID NOs: 1-11. Loss of E-cadherin expression as indicated by level of fluorescence is an indication of the loss of the epithelial phenotype. Similar analysis may be conducted with other markers of loss of epithelial phenotype, including loss of cytokeratins and apical actin-binding transmembrane protein-1 (MUC-1). Loss of E-cadherin expression is a universal feature of EMT, regardless of the initiating stimulus (Hay E D, Acta Anat., 1995, 154:8-20). Reversal of the mesenchymal phenotype may be observed by increased production of E-cadherin (Vanderburg C R, Acta Anat, 1996, 157:87-104).

FIGS. 2-16 are fluorescence micrographs showing the effect of the tested compounds (SEQ ID NOs: 1-11) on the level of E-cadherin (marker of the epithelial phenotype). FIG. 2 shows the fluorescence due to immunofluorescent staining of E-cadherin expressed in HK-2 cells exposed to culture medium only, while FIG. 3 (cells in the presence of 100 mM D-glucose) shows the D-glucose-induced loss of E-cadherin expression (as observed by immunofluorescent staining of cells).FIGS. 4-16 show the immunofluorescence for HK-2 cells treated with 100 mM D-glucose and 100 uM of compound SEQ ID NO: 1 (TFA, acetate, and chloride salts), and of compounds SEQ ID NOs: 2-11, respectively. Because it is not clear in all cases how to evaluate the effect of a compound from a fluoromicrographs, a colorimetric analysis method was developed (see Methods and Materials for Examples 1-4, Section C) and the results of that analysis are shown in Table 3 and FIG. 1.

FIG. 17 depicts the STZ experimental protocol behind the study discussed in Example 2. Experiments were performed on out-bred CD 1 mice maintained on a normal diet under standard animal house conditions. Mice were given a single intraperitoneal injection of Streptozotocin (STZ) in sodium citrate buffer (pH 4.5) at a dose of 200 mg/kg. Blood glucose was measured by tail vein sampling using the glucose oxidase enzymatic test (Medisense glucometer, Abbott Laboratories, Bedford, Mass.). Diabetic nephropathy was evaluated in groups of mice killed at the end of 5 or 6 months (vehicle control groups) or 6 months (THR123 or BMP-7 group) after STZ injection. The Details of experimental design are shown in FIG. 17. A group of STZ injected mice received oral administration of Thrasos compound daily at a dose of 5 mg/kg body weight for a month, from month 5 to month 6 after STZ injection. BMP-7 was administered intraperitoneally at a dose of 300 μg/kg body weight from month 1 to month 6.

FIGS. 18-22 are representative photomicrographs of tissues of the mice in the study outlined in FIG. 17 and discussed in Examples 2 and 3, and FIGS. 24-29. More in particular, these are representative photomicrographs of kidney sections from control mice, mice at 5 months after diabetic nephropathy induction, from mice at 6 months after diabetic nephropathy induction, from mice at 6 months after diabetic nephropathy induction who were treated with BMP7 from 1 to 6 months after induction and from mice at 6 months after diabetic nephropathy induction who were treated with THR-123 compound (SEQ ID NO 1) from 1 to 6 months after induction.

FIG. 18: Representative histology of kidney sections from control mice (STZ minus, n=5)

FIG. 19: Representative histology of kidney sections from mice (n=6) at 5 months after STZ-induced diabetic nephropathy based on study outlined in FIG. 17 and discussed in Examples 2 and 3, and FIGS. 24 to 29.

FIG. 20: Representative histology of kidney sections from mice (n=10) at 6 months after diabetic nephropathy induction based on the study outlined in FIG. 17 and discussed in Examples 2 and 3, and FIGS. 24 to 29.

FIG. 21: Representative histology of kidney sections from mice (n=4) at 6 months after diabetic nephropathy induction who were treated with BMP-7 from 1 to 6 months after induction, based on the study outlined in FIG. 17 and discussed in Examples 2 and 3, and FIGS. 24 to 29.

FIG. 22: Representative histology of kidney sections from mice (n=9) at 6 months after diabetic nephropathy induction who were treated with THR-123 (SEQ ID NO 1) from 5 to 6 months after induction, based on the study outlined in FIG. 17 and discussed in Examples 2 and 3, and FIGS. 24 to 29.

FIG. 23 depicts quantitation of H&E (hematoxylin and eosin) and Masson's trichrome stains of kidney sections identified in FIGS. 18 through 22. Masson's Trichrome-stained sections were used to analyze the accumulation of collagen in the interstitium. With this stain, collagen is colored blue and cells are red. Figure shows a substantial increase in interstitial fibrosis in mice kidneys at 5 and 6 months after diabetic nephropathy induction. However, the collagen accumulation indicating interstitial fibrosis is markedly reduced in mice after treatment with THR-123 (SEQ ID NO 1) for 1 month (from month 5 to month 6) after diabetic nephropathy induction. The analysis method is discussed in Method C under Examples 1-4. The effect of treating with THR-123 for the last month is similar to the effect observed with BMP-7 treatment for the last 5 months and large enough to suggest reversal of fibrosis.

FIG. 24 depicts that the net increases in FSP-1 (fibroblast secretory protein-1), a mesenchymal marker expression were 27 and 29 times at 5 and 6 months after diabetic nephropathy induction, respectively, when compared to that observed for the normal animals. This increase was markedly reduced in mice treated with THR-123 (SEQ ID NO 1) for one month (from month 5 to month 6) after diabetic nephropathy induction. After a one month treatment, THR-123 reduced the marker concentration to less than 1/10 the levels at 5 and 6 months.

FIG. 25 depicts that mice showed high percent damaged tubules at 5 and 6 months after diabetic nephropathy induction compared to the normal animals. The tubular damage, however, was markedly reduced in mice treated with THR-123 (SEQ ID NO 1) for one month (from month 5 to month 6) after diabetic nephropathy induction. Thus, exposure to STZ results in the progressive loss of intact tubules in the kidney cortex. Administration of BMP starting after one month halts the progression and administration of THR-123 during the last month of the study appears to reverse the loss of tubules.

FIG. 26 depicts that the relative interstitial volume of kidneys increased significantly in mice at 5 and 6 months after diabetic nephropathy induction, and it was markedly reduced in mice treated with THR-123 (SEQ ID NO 1) for one month (from month 5 to month 6) or BMP-7 (from month 1 to month 6) after diabetic nephropathy induction. Thus, treatment with BMP7 starting after the first month halts progression of the increase in interstitial volume, and treatment with THR-123 (SEQ ID NO: 1) appears to reverse the progress.

FIG. 27 depicts that the glomerular surface increased significantly in mice at 5 and 6 months after diabetic nephropathy induction, and it was markedly reduced in mice treated with THR-123(SEQ ID NO 1) for one month (from month 5 to month 6) or BMP-7 (from month 1 to month 6) after diabetic nephropathy induction. Thus, treatment with BMP7 appears to slow deterioration of the glomeruli, but THR-123 (SEQ ID NO: 1) appears to have no effect.

FIG. 28 depicts that the mesangial matrix increased in mice at 5 and 6 months after diabetic nephropathy induction. This increase was significantly reduced in mice treated with THR-123 (SEQ ID NO 1) for one month (from month 5 to month 6) or BMP-7 (from month 1 to month 6) after diabetic nephropathy induction. Treatment with SEQ ID NO: 1 reduced the level to 37% at 6 months.

FIG. 29 depicts that BUN levels increased in mice at 5 and 6 months after diabetic nephropathy induction. However, in mice treated with THR-123 (SEQ ID NO 1) for one month (from month 5 to month 6) or BMP-7 (from month 1 to month 6) after diabetic nephropathy induction, the BUN levels dropped to those of control animals, suggesting a significant improvement in kidney function after THR-123 treatment. Thus, BMP7 administered over the last 5 months of the study kept the BUN level increase to 2%, whereas treatment with THR-123 (SEQ ID NO: 1) for the last month before sacrifice reduced the increase in BUN from 85% at 5 months to 12%.

FIG. 30 provides a diagram depicting the structure of a peptide of the invention, designated as THR-123. This compound corresponds to SEQ ID NO: 1 of Table 1. The diagram further includes on the left a three dimensional stick model on the structure of hBMP7 and indicates the location of a conserved loop in hBMP7 that is mimicked by THR-123 at least in part through the strategic placement of the a disulphide linkage between the cysteine at position 1 and the cysteine at residue 11.

FIG. 31 provides a table indicating a comparison between THR-123 (SEQ ID NO: 1) and BMP-7 for binding to Type I and Type II BMP receptors. Like BMP-7, THR-123 binds to both ALK2 and ALK3 (type I) and BMPR-II (type II) BMP receptors. However, BMP-7, but not THR-123 binds to ALK6 (type I) receptor. Of particular note is that unlike BMP7, THR-123 does not bind to the ALK6 BMP type I receptor ECD.

FIG. 32 shows that THR-123 (SEQ ID NO: 1) induces Smad 1/5/8 phosphorylation and nuclear translocation, suggesting that the compound is an agonist of BMP signaling. Human renal proximal tubule epithelial cells (HK-2) were incubated in the presence (right panel) and absence (left panel) of THR-123. The cells were washed, and then incubated with the primary antibody against phospho Smad 1/5/8, followed by immunostaining with a fluorescently labeled secondary antibody. The significance of pSmad 1/5/8 in renal fibrosis is tied to BMP7 suppression of TGF-β-dependent profibrotic pathways, which are central to renal fibrotic injury. Specifically, BMP7 suppression of such TGF-β-dependent profibrotic pathways is mediated in part by the activation of downstream BMP target proteins, Smad 1, 5, and 8. (Manson S R et al., J. Urol. 85:2523-30, 2011).

FIG. 33 shows an increase in interstitial volume after unilateral ureteral obstruction (UUO) is apparent in the figure. UUO animals showed a 3 fold expansion of the interstitial space. In the animals given BMP-7 or THR-123 (SEQ ID NO: 1), the expansion of the interstitial space was significantly reduced, suggesting prevention of interstitial fibrosis by Thrasos compound.

FIG. 34 shows the expansion of the renal interstitium after unilateral ureteral obstruction (UUO) was further examined by analyzing the deposition of collagen (indicative of fibrosis). Hydroxyproline content, a measure of total collagen, increased 3-fold in the vehicle-treated UUO kidneys compared with sham-operated kidneys. THR-123 (SEQ ID NO: 1) and BMP-7 effectively decreased the UUO-induced increased hydroxyproline content, suggesting that THR-123 ameliorates UUO-induced renal fibrosis.

FIG. 35 provides a bar graph showing a comparison of the effects of THR-123 (SEQ ID NO: for “THR-C”) and the specific inhibitor SB 203580 on the level of phosphorylation of p38 MAPK in human renal tubule epithelial cells (HK-2). The significance of p38 MAPK is that this protein is implicated as a non-Smad-dependent pathway for TGF-β-dependent EMT. Other non-Smad-dependent pathways implicated in TGF-β-dependent EMT include RhoA, Ras, PI3 kinase, Notch, and Wnt signaling pathways. The results show that THR-123 effectively inhibited basal p38 phosphorylation in HK-2 cells. A specific inhibitor, SB203580 and BMP-7 which served as positive controls inhibited p38 phosphorylation as expected. THR-123 at a lower concentration of 1 uM was as potent as 10 uM of the specific inhibitor in the assay.

FIG. 36 provides a bar graph showing a comparison of the effects of THR-123 (SEQ ID NO: 1 or “THR-C”) and the specific inhibitor SB 203580 on the level of phosphorylation of p38 MAPK resulting from TNF-α stimulation. It has been shown that regulation p38 MAPK activity by pro-inflammatory factors has implications in fibrosis. The results indicate that TNF alpha induced p38 phosphorylation in HK-2 cells and that the induced phosphorylation by TNF alpha was effectively inhibited by THR-123 alone or in combination with SB 203580.

FIG. 37 provides a bar graph showing a comparison of the effects of THR-123 (or “THR-1405”) and the specific inhibitor SB 203580 on the level of TNF-α-induced IL-6 production, an inflammatory marker, in human renal tubule epithelial cells (HK-2). The results indicate that TNF alpha stimulated IL-6 production in HK-2 cells. The addition of THR-123 or SB 203580 alone significantly reduced TNF alpha-induced IL-6 production by HK-2 cells. Addition of both in combination caused greater decrease in IL-6 production compared with THR-123 alone. These results suggest blockade of p38 activation by THR-123 inhibits cellular inflammation which is an important determinant of the progression of renal fibrosis.

FIG. 38 Tumor necrosis factor-a (TNF-a) production via p38 mitogen-activated protein kinase (MAPK) is one of the pivotal mechanisms in the development of AKI induced by a nephrotoxic agent, cisplatin (Ramesh, G and Reeves, W B. Am J Physiol Renal Physiol 289:F166-F174, 2005). Therefore THR-123 was further examined to determine if the compound is capable of inhibiting cisplatin-induced nephrotoxicity in animals. The upper right and left panels in the figure show Kidney sections immunostained for ICAM-1 expression and the lower right and left panels show immunostaining for the presence of macrophages. The kidney sections in the left column were treated with cisplatin alone and the right column panels were treated with cisplatin and THR-123. Arrows in the upper panel indicate ICAM-1 expression which was reduced by THR-123. Arrows in the lower panel indicate infiltration of macrophages as detected by Mac CD-68 staining, which was reduced by THR-123. The results indicate that THR-123, capable of inhibiting p38 MAPK in injured renal PTEC, was able to inhibit tubular infiltration of macrophages, and thus inflammation in cisplatin-induced nephrotoxicity in rats.

FIG. 39 demonstrates that epithelial-mesenchymal transition (EMT) in renal proximal tubule epithelial cells (HK-2) is prevented by THR-123. Exposure of HK-2 cells to high glucose results in a significant loss of E-cadherin expression (lower panel) suggesting that EMT is induced. THR-123 effectively prevents D-glucose (50 mM) induced loss of epithelial phenotype (as assessed by the expression of E-cadherin) in renal PTEC. These results suggest that under hyperglycemic conditions (diabetic conditions) Thrasos compound is capable of preventing Epithelial-Mesenchymal-Transition (EMT) process, an essential mechanism involved in tubulo-interstitial fibrosis.

FIG. 40 demonstrates the effect of orally administered THR-123 on advanced diabetic nephropathy model of chronic kidney disease in mice. When mice were treated orally with THR123 for one month (from month 5 to month 6) after diabetic nephropathy induction, the compound reduced renal fibrosis (bottom right panel) and decreased interstitial volume (bar graph).

FIG. 41A-41C demonstrates that THR-123 failed to induce osteoblastic differentiation of pluripotent stem cells (C3H10T1/2). Murine pluripotent mesenchymal stem cells (C3H10T1/2) treated with medium alone, BMP-7 or THR-123 were stained for alkaline phosphatase activity, an osteogenic marker. No staining of cells was observed when treated with medium alone (control, panel FIG. 41A) or with THR-123 (panel FIG. 41B). BMP-7, 2 ug/mL (panel FIG. 41C) which served as a positive control induced osteoblastic differentiation of pluripotent stem cells, as stained for alkaline phosphatase activity. Cells were counterstained with hematoxylin.

FIG. 42A-42Z. The role of endogenous Alk-3 expression in the renal tubules for kidney fibrosis. FIG. 42A. Quantitative real time PCR. Total RNA was isolated from kidneys of C57BL/6 mice before (day 0) and after induction of nephrotoxic serum nephritis (1 week, 3 weeks, 6 weeks and 9 weeks after immunization). Quantitative RT PCR was performed using specific primer set for indicated genes. The graph displays relative expression against 18sRNA at each time point. B-D. Representative picture of Masson's trichrome staining of control or nephrotoxic serum treated kidneys. Magnification ×100. FIG. 42E-FIG. 42G Representative picture of corresponding kidneys (FIG. 42B-FIG. 42D) that were labeled with antibodies specific to phosphorylated Smad1, indicative of active BMP signaling. Magnification ×200. FIG. 42H. Schematic illustration. Mice which express Cre-recombinase under the control of the γGT promoter were bred to mice in which the LacZ reporter gene is separated from the Rosa26 promoter by a floxed STOP cassette to generate in γGT-Cre; R26R-STOP-LacZ reporter mice. I-J. Beta-galactosidase staining. Kidneys of control R26R-STOP-LacZ mice (FIG. 42I) and γGT-Cre; R26R-STOP-LacZ reporter mice (FIG. 42J) were enzymatically stained to detect β-galactosidase activity (blue precipitate) counter stained with eosin. Arrows in panel indicate representative LacZ staining. Magnification ×400. FIG. 42K. Schematic illustration. Mice conditionally lacking Alk-3 in kidney tubular epithelial cells (γGT-Cre,Alk-3^{flox/flox}) were generated by γGT-Cre mice crossbred with mice carrying floxed Alk-3 alleles. L-M. Alk-3 immunohistochemistry analysis. Robust expression of Alk-3 in control Alk-3^{flox/flox} mice (FIG. 42L). No tubular Alk-3 protein expression was detected in γGT-Cre; Alk-3^{flox/flox} mice (FIG. 42M). FIG. 42N-FIG. 42U. Histopathology. γGT-Cre; Alk-3^{flox/flox} mice and littermate control mice (Alk-3^{flox/flox}) were challenged with nephrotoxic serum. Representative picture of Masson's trichrome staining kidneys at magnification ×200. FIG. 42V. Quantification of fibrosis in NTN kidney. Masson's trichrome staining pictures are analyzed by imageJ software and fibrosis area was quantified. In each time points 4-6 mice were analyzed. FIG. 42W. Blood urea nitrogen measurement in day 60 of NTN in γG-Cre; Alk-3^{flox/flox} mice (n=5) and littermate control mice (n=3). FIG. 42X, FIG. 42Y. E-cadherin/FSP1 immunolabeling of kidney in control (Alk3^{flox/flox}) and γGTCre; Alk3^{flox/flox} mice. FIG. 42Z. Percent of E-cadhrin/FSP1 double positive tubule was assessed by counting the number of double-labeled tubules, 500 tubules per slide, 5 slides per experimental group. Data are expressed as mean±s.e.m. in the graph.

FIG. 43A-FIG. 43C Increased tubule p-smad2 accumulation in γGT-Cre; Alk3^{flox/flox} mice. FIG. 43A, FIG. 43B. phospho-smad2 (p-smad2) immunolabeling of kidney in Alk3^{flox/flox} and gGTCre; Alk3^{flox/flox} mice. FIG. 43C. Percent of p-smad2 positive tubule was assessed in tubules, 500 tubules per slide, 5 slides per experimental group. Data are expressed as mean±s.e.m. in the graph.

FIG. 44A-FIG. 44D Macrophage accumulation in γGT-Cre; Alk3^{flox/flox} mice. Frozen section was labeled for macrophage using Mac-1 antibody and immunofluorescence analysis was performed by fluorescence microscopy. In the kidney without disease (FIG. 44A and FIG. 44B) minor macrophage were found. In NTN kidney of Alk3^{flox/flox} mice, macrophages are accumulated (FIG. 44C), and such macrophage accumulation is prominent in the NTN kidney of □GT-Cre; Alk3^{flox/flox} mice (FIG. 44D). The representative pictures from 5 independent experiments are shown.

FIG. 45A-FIG. 45G Pharmacokinetics of THR-123. FIG. 45A. BMP7 structure figure with the residue weights resulting from the analysis mapped on to it. FIG. 45B, FIG. 45C. Radio-ligand receptor binding assays specific for individual type I receptors, Alk-3 (FIG. 45B) and Alk-6 (FIG. 45C). Highly purified extra-cellular domain (ECD) of Alk-3 or Alk-6 (expressed as a fusion protein with Fc domain) served as a receptor. In each assay purified receptor was immobilized on each well and peptide analog or unlabeled BMP7 was added, followed by ¹²⁵I-labeled BMP7. Radiolabeled BMP7 complex was counted in an auto-gamma counter. Results were expressed as the mean±s.e.m. Unlabeled BMP7, which served as a positive control in both assays, gave linear dose-related response curves. FIG. 45D, FIG. 45E. Concentration of THR-123 in systemic circulation of Wistar rats following iv injection of THR-123 (¹²⁵I-Tyr) via the tail vein determined by total radioactivity measurement. The alpha phase (FIG. 45D) accounts for approximately 90% of the injected dose and has very short half-life. The beta phase (FIG. 45E) accounts for the remaining 10% of the injected dose and has a much longer half-life of 55-58 min. FIG. 45F. Tissue distribution of ¹²⁵I-THR-123. Six hours after intravenous administration of ¹²⁵I labeled-THR-123 in rats at a dose of 6.25 mg/kg-body weight, tissues were harvested and analyzed by automatic gamma well counter. Majority of radioactivity was localized in kidney and bladder. FIG. 45G. Oral administration of THR-123 and elimination of THR-123 from the body. Radioactivity of orally administrated ¹²⁵I labeled-THR-123 at a dose of 5 mg/kg body weight was localized in the kidney between 1 and 6 hours after administration and peaked at around 3 hours. The radioactivity from ¹²⁵I labeled-THR-123 was completely cleared from the kidney 24 hours after administration.

FIG. 46 In vitro stability of THR-123. THR-123 was spiked into freshly harvested rat blood (male Sprague-Dawley, 0.35 kg BW) and plasma, and PBS-mannitol buffer solution at a final concentration of 0.1 mg/mL. Blood, plasma and buffer master tubes were incubated at 37° C. for up to 6 h and duplicate samples of 500 μl (blood) and 250 μl (plasma and blood) were collected for analysis at 0, 7.5, 15, 30, 60, 120, 240 and 360 min. Samples were analyzed for THR-123 using an LC-MS-MS method having a limit of detection of 1 μg/ml. THR-123 was slowly degraded in plasma with a half-life of 358 min., and more rapidly in blood where the half-life was only 70 min. In the PBS-mannitol buffer, there was no observable degradation over 400 min.

FIG. 47A-FIG. 47C Anti-inflammatory activity of THR-123. PTEC-derived HK-2 (HK-2) cells were culture on 24-well plate (30,000 cells/well). Cells are exposed to K-SFM medium alone or TNF-α (5 ng/ml). Twenty hours after TNF-α incubation, cells are washed twice by pre-warmed culture media and subsequently cells are incubated with various concentration of THR-123 or BMP7 for 60 hours. At the end of incubation, culture medias are harvested and ELISA analysis are performed. FIG. 47A: IL-6, FIG. 47B: IL-8 and FIG. 47C: ICAM-1 results are shown. Analyze are performed in triplicates and data are shown as mean±s.e.m. in the graph.

FIG. 48A-FIG. 48D THR-123 inhibits TGF-β-induced apoptosis in NP-1 cells. NP-1 cells are incubated with TGF-β (3 ng/ml) for 24 h in the presence of indicated molecules. Apoptosis was analyzed by Annexin V labeling (Roche). Representative merged picture (Green: Annexin V and bright field image) of cells treated with TGF-β only (FIG. 48A), TGF-β+BMP-7 (1 μg/ml) (FIG. 48B), TGF-β+THR-123 (10 μM) (FIG. 48C) and TGF-β+ctrl peptide (FIG. 48D). TGF-β increased apoptosis and BMP-7 and THR-123 decreased apoptosis.

FIG. 49A-FIG. 49D THR-123 inhibits Hypoxia-induced apoptosis in NP-1 cells. NP-1 cells are incubated with hypoxia (2.5% O₂) for 24 h in the presence of indicated molecules. Apoptosis was analyzed by Annexin V labeling (Roche). Representative merged picture (Green: Annexin V and bright field image) of cells treated with hypoxia only (FIG. 49A), hypoxia+BMP-7 (1 μg/ml) FIG. 49(B), hypoxia+THR-123 (10 μM) (FIG. 49C) and hypoxia+ctrl peptide (FIG. 49D). Hypoxia increased apoptosis; BMP-7 and THR-123 decreased apoptosis.

FIG. 50A-FIG. 50D THR-123 inhibits Cisplatin-induced apoptosis in human proximal tubule epithelial cells (HK2). Immortalized human proximal tubular epithelial-derived HK-2 (Human Kidney-2) cells are passaged on 24-well plates (˜25,000 to 30,000 cells/well). The cells are exposed either K-SFM media alone or K-SFM medium containing THR-123. BMP7 serves as a positive control of experiment. Two hours after incubation, cells are exposed to cisplatin for 60 hours (FIG. 50A-FIG. 50C). FIG. 50D. Cells are exposed to cisplatin for 6 hours and subsequently THR-123 was added into the media. Apoptosis is determined by staining of AnnexinV-FITC Apoptosis detection kit (TACS Annexin V-FITC) (R&D Systems), followed by fluorescence microscopy. Final concentration: THR-123 250 μM, BMP-7 1 μg/ml, cisplatin 10 μm.

FIG. 51A-FIG. 51J THR-123 inhibits epithelial-mesenchymal transition in NP-1 cells. FIG. 51A-FIG. 51E: Bright field image. Cells exposed to TGF-β (3 ng/ml each in serum-free DMEM medium) with EGF for 48 h undergo EMT and showed marked elongation of the cell shape when compared to control cells (FIG. 51A, FIG. 51B). Co-incubation of either BMP-7 (1 μg/ml) or THR-123 (10 μM) prevents these phenotypic changes (FIG. 51C, FIG. 51D). Control peptide showed no effect on TGF-β-induced EMT (FIG. 51E). F-J. E-cadherin immunofluorescence labeling. NP-1 cells expressed E-cadherin in cell border (FIG. 51F). Cells exposed to TGF-β for 48 h exhibited marked reduction of E-cadherin levels when compared to control cells (FIG. 51F, FIG. 51G). Co-incubation of either BMP-7 (1 μg/ml) or THR-123 (10 μM) prevents E-cadherin loss (FIG. 51H, FIG. 51I). Control peptide showed no effect on TGF-β-induced EMT (FIG. 51J). Representative results are shown.

FIG. 52A-FIG. 52E THR-123 inhibits epithelial-mesenchymal transition in MCT cells. Inhibition of EMT by THR-123. Cells exposed to TGF-β (2.5 ng/ml each in serum-free DMEM medium) for 48 h undergo EMT and showed marked elongation of the cell shape (FIG. 52B). Co-incubation of THR-123 (10 μM) prevents these phenotypic changes (FIG. 52C). FIG. 52D, FIG. 52E qPCR analysis for mesenchymal marker CTGF (FIG. 52D) and Snail1 (FIG. 52E). Total RNA was extracted from cells. 1 μg of total RNA was used for generating complementary cDNA and subsequently qPCR was performed. N=3. Data was expressed as mean±s.e.m. in the graph.

FIG. 53A-FIG. 53L Reversal of EMT by THR-123 in NP-1 cells A-E: Inverted microscopic pictures. FIG. 53A. Basal polygonal epithelial nature of NP1 cells. FIG. 53B. Incubation with TGF-β and EGF for 48 h induced EMT and showed elongated, spindle shaped cells. FIG. 53C, FIG. 53D. BMP 7 (1 μg/ml) (FIG. 53C) or THR-123 (10 μM) (FIG. 53D) were added to mesenchymal like cells induced by TGF-β and EGF for the additional 48 hrs. The cells show reversal of the morphology and show polygonal epithelial nature again (FIG. 53C, FIG. 53D). FIG. 53E. Ctrl peptide did not reverse EMT. FIG. 53F. Ratio of length to width was calculated in reversal experiment. Five representative pictures of the cells in different areas of the well, were taken by using the inverted microscope. A total of 100 cells were analyzed (20 cells per picture). Epithelial cell morphology is characterized lower and mesenchymal cells exhibit higher ratio. The data are presented mean+s.e.m. in the graph. FIG. 53G. Basal length to width ratio NP-1 cells exhibited 1.4+0.2 (mean±SD). Mean plus one SD was estimated as epithelial characteristics and the percentage of epithelial characteristics in each experimental setting were estimated. 18% of BMP7 and 24% of THR-123 treated NP-1 cells regained epithelial characteristics. FIG. 53H-FIG. 53L. Immunofluorescence picture for E-cadherin. TGF-β incubation decreased E-cadherin levels when compare to untreated cells (FIG. 53H, FIG. 53I). BMP-7 (FIG. 53J) and THR-123 (FIG. 53K) reversed E-cadherin expression in TGF-β-incubated NP-1 cells. Ctrl peptide exhibited no effect on E-cadherin levels (FIG. 53L). Representative results from three independent experiments are shown.

FIG. 54A-FIG. 54H THR-123 reverses TGF-β-induced EMT in MCT cells. FIG. 54A-FIG. 54E. Cell were induced EMT by 48 h incubation of TGF-β and EGF (FIG. 54E). After induction of EMT, cells are incubated with either BMP-7 (1 μg/ml) (FIG. 54C), THR-123 (10 μM) (FIG. 54D) or control peptide (FIG. 54E) for an additional 48 h. Reversal of EMT was observed (FIG. 54C, FIG. 54D). Ctrl peptide exhibited no effect (FIG. 54E). FIG. 54F. Ratio of length to width was calculated in reversal experiment. 5 representative pictures of the cells in different areas of the well, were taken by using the inverted microscope. A total of 100 cells were analyzed (20 cells per picture). Epithelial cell morphology is characterized lower and mesenchymal cells exhibit higher ratio. The data are presented mean±s.e.m. in the graph. FIG. 54G: 3 or less in length to width ratio in MCT cell was estimated as epithelial characteristics and the percentage of epithelial characteristics in each experimental setting are estimated. 52% of BMP7 and 41% of THR-123 treated MCT cells regained epithelial characteristics. Experiments were repeated three times.

FIG. 55A-FIG. 55D The effect of THR-123 on acute tubular damage induced by ischemia reperfusion injury. Ischemia reperfusion injury (IRI) was induced by the clamping of the left renal pedicle for 25 minutes. After ischemia reperfusion injury the mice were given THR-123 orally (5 mg/kg/day) or PBS till the day of sacrifice. FIG. 55A. kidney histology after ischemia re-perfusion in phosphate buffered saline treated mice shows severe acute tubular necrosis. Arrows indicate necrotic tubules. FIG. 55B. Kidney histology in mice treated with simultaneous administration of THR-123, following the ischemia reperfusion procedure shows mild tubular necrosis. FIG. 55C. Percentage renal tubular necrosis in THR-123 treated mice is significantly less than the phosphate buffer treated mice. Ten fields in each group were analyzed. FIG. 55D. Blood urea nitrogen estimated by quantichrome colorimetric urea assay at day 7 IRI. No difference in the all groups. PBS group (n=5) and THR-123-treated group (n=4) are analyzed. Data are shown as mean±s.e.m. in the graph.

FIG. 56A-FIG. 56J The effect of THR-123 on mice with unilateral ureteral obstruction (UUO) mice. At the day of UUO, BMP-7 (300 μg/Kg/every other day, intraperitoneal administration) or THR-123 (5 mg/Kg/day, oral or intraperitoneal administration) are initiated. FIG. 56A-FIG. 56D: Masson's trichrome staining of normal kidney, day 5 UUO kidney. FIG. 56A. normal kidney section. FIG. 56B. day 5 UUO mice kidney shows normal glomeruli, tubular atrophy, tubular dilatation and interstitial inflammation. FIG. 56C, FIG. 56D. Kidney of UUO mice treated with THR-123 orally at 5 mg/kg (FIG. 56C) or 15 mg/kg (FIG. 56D) shows less tubular atrophy, tubular dilatation and interstitial inflammation. FIG. 56E. Morphometric analysis for relative interstitial volume. Interstitial volume was calculated by the point counting method. FIG. 56F-FIG. 56I. Masson's trichrome staining of normal kidney, day 7 UUO kidney. The UUO mice treated with BMP7-intraperitoneally (FIG. 56G) or THR-123-intraperitoneally (FIG. 56H)/orally (I) show less kidney interstitial volume when compared to PBS treated (FIG. 56F). FIG. 56I. Morphometric analysis for relative interstitial volume. Interstitial volume was calculated by the point counting method. For the quantification of tubulo-interstitial lesions, 8 fields per kidney were analyzed in each mouse. Normal mice (n=4), day 5 UUO without treatment (n=4), day 5 UUO-treated with 5 mg/kg THR-123 (n=4), day 5 UUO-treated with 15 mg/kg THR-123 (n=4), day 7 UUO-treated with PBS (n=8), -treated with BMP (n=8) and -treated with THR-123 (n=7 in both i.p. and oral). Data are shown as mean±s.e.m. in the graph (FIG. 56J).

FIG. 57A-FIG. 57H The effect of THR-123 on mice with unilateral ureteral obstruction (UUO) mice (H&E staining). On the day UUO was performed, treatments of BMP7 (300 μg/Kg/every other day, intraperitoneal administration) or THR-123 (5 mg/Kg/day, oral or intraperitoneal administration) are initiated. FIG. 57A. Normal kidney section. FIG. 57B. Day 5 UUO mice FIG. 57C, FIG. 57D. Kidney of UUO mice treated with THR-123 orally at 5 mg/kg (FIG. 57C) or 15 mg/kg (FIG. 57D) E-H. Day 7 UUO kidney histology. When compared to PBS-treated UUO kidney at day 7 (FIG. 57E), the UUO mice treated with BMP7-intraperitoneally (FIG. 57F) or THR-123-intraperitoneally (FIG. 57G)/orally (FIG. 57H) exhibit preserved tubules in the kidney. Normal mice (n=4), day 5 UUO without treatment (n=4), day 5 UUO-treated with 5 mg/kg THR-123 (n=4), day 5 UUO-treated with 15 mg/kg THR-123 (n=4), day 7 UUO-treated with PBS (n=8), -treated with BMP (n=8) and -treated with THR-123 (n=7 in both i.p. and oral).

FIG. 58 Gene expression analysis for the fibrosis markers in mice with UUO. Quantitative RT-PCR analysis for fibronectin-EIII and collagen type-I in normal kidney and day 7 UUO kidney with or without indicated treatment.

FIG. 59A-FIG. 59M AA-123 Reverses Renal Fibrosis in Mice with Nephrotoxic Serum Nephritis. FIG. 59A-FIG. 59D. Representative Masson's trichrome staining picture of kidney sections from untreated control mice (FIG. 59A); 6 weeks NTN (FIG. 59B); 9 weeks post NTN (FIG. 59C); and 9 weeks NTN with THR-123 administered starting at 6 weeks NTN (FIG. 59D), Original magnification ×200. FIG. 59E-FIG. 59G. Morphometric analysis from control mice (0 weeks) (n=5), mice after 1 (n=6), 3 (n=8), and 6 weeks following induction of NTN (n=6), and mice at 9 weeks NTN with THR-123 administered starting 6 weeks NTN (THR-123 (6-9W) (n=6)), assessing percent of glomerulosclerosis score (FIG. 59E), tubular atrophy index (FIG. 59F), and fibrosis index (FIG. 59G). FIG. 59H. Blood Urea Nitrogen measurement for mice 6 weeks NTN (n=3), 9 weeks NTN (n=5), and 9 weeks NTN with THR-123 administered starting at 6 weeks NTN (n=5) FIG. 59I-FIG. 59L. E-cadherin/FSP1 immunolabeling of kidney from control untreated mice (FIG. 59I), 6 weeks NTN (FIG. 59J), 9 weeks NTN (FIG. 59K), and 9 weeks NTN with THR-123 administered starting at 6 weeks NTN (FIG. 59L). Representative results are shown. FIG. 59M. Percent of E-cadherin/FSP1 double positive tubule was assessed by counting the number of double-labeled tubules, 500 tubules per slide, 5 slides per experimental group. Data are expressed as mean±s.e.m. in the graph.

FIG. 60A-FIG. 60D Light Microscopy (H&E) Analysis of the kidneys from mice with Nephrotoxic serum nephritis. Representative histological H&E staining of kidneys from untreated control mice (FIG. 60A); 6 weeks NTN (FIG. 60B), 9 weeks NTN (FIG. 60C); and 9 weeks NTN with THR-123 administered starting 6 weeks post NTN induction (FIG. 60D), magnification ×200.

FIG. 61 Gene expression analysis for fibrosis markers in the mice with nephrotoxic serum nephritis. Fold gene expression via quantitative real-time PCR measurements for fibronectin (FN-EIII) and type I collagen (COL-I) in kidneys of control mice (0 weeks NTS) (n=5), mice after 1 (n=6), 3 (n=8), and 6 weeks NTN (n=6), and mice at 9 weeks NTN with THR-123 administered starting 6 weeks post NTN induction (THR-123 (6-9W) (n=6)).

FIG. 62A-FIG. 62E Macrophages Analysis in mice with Nephrotoxic Serum Nephritis. FIG. 62A-FIG. 62C Mac-1 immunolabeling of kidney from untreated control mice (FIG. 62A), 6 weeks NTN (FIG. 62B); and 9 weeks NTN (FIG. 62C), and 9 weeks NTN treatment with THR-123 administered starting 6 weeks after induction of NTN (FIG. 62D), magnification ×400. FIG. 62E. The number of macrophages per field of view (×400 magnification) was assessed by counting positively labeled cells in 5 random fields of view per slide, with 5 slides per experimental group. Data are shown as mean+s.e.m. in the graph.

FIG. 63A-FIG. 63D phospho-smad1/5 labeling in NTN. FIG. 63A-FIG. 63C. Frozen kidney sections of indicated group of animals were labeled with phospho-smad1/5 (p-smad1/5) antibody followed by FITC-conjugated secondary antibody. p-smad1/5 levels are analyzed by fluorescence microscopy. Arrows in panel (FIG. 63C) indicate nuclear accumulated p-smad1/5. FIG. 63D. Quantification of p-smad1/5 level. The number of p-smad1/5 per field of view (×400 magnification) was assessed by counting positively labeled cells in 5 random fields of view per slide, with 5 slides per experimental group. Data are shown as mean±s.e.m. in the graph.

FIG. 64a-FIG. 64N AA-123 inhibits Renal Fibrosis in the COL4A3 Deficient mice. FIG. 64A-FIG. 64C. Representative histological PAS staining of glomeruli from 16 weeks old wild type (FIG. 64A); 16 weeks old COL4A3−/− (FIG. 64B), and 16 weeks old COL4A3−/− mice treated with THR-123 (FIG. 64C), original magnification ×400. D-F. Representative histological Masson's trichrome staining of kidneys from 16 weeks old wild type (FIG. 64D); 16 weeks old COL4A3−/− (FIG. 64E), and 16 weeks old COL4A3−/− mice treated with THR-123 (FIG. 64F), original magnification ×100. FIG. 64G-FIG. 64I. Morphometric analyses from 16 weeks old wild type (n=5), COL4A3−/− (n=5) and COL4A3−/− mice treated with THR-123 (n=5) assessing percent normal glomeruli (FIG. 64G), tubular damage index (FIG. 64H), relative interstitial volume (FIG. 64I). FIG. 64J. Blood urea nitrogen measurement for 20 weeks old wild type (n=5), 16 weeks old COL4A3−/− (n=5) and COL4A3−/− mice treated with THR-123 (n=5). FIG. 64K-FIG. 64M. FSP1/E-cadherin immunolabeling of kidney. Representative results are shown. FIG. 64N. Percent of E-cadherin/FSP1double positive tubule, 500 tubules per slide, 5 slides per experimental group. Data are expressed as mean+s.e.m. in the graph.

FIG. 65A-FIG. 65D Macrophage Analysis in the COL4A3 Deficient Mice. FIG. 65A-FIG. 65C Mac-1 immunolabeling of kidney from 16 weeks old wild type (FIG. 65A), COL4A3−/− (FIG. 65B) and COL4A3−/− mice treated with THR-123 (FIG. 65C). Original magnification is ×400. FIG. 65D. Morphometric analysis of number of macrophages per field of view (×400 magnification) was assessed by counting positively labeled cells in 5 random fields of view per slide, with 5 slides per experimental group. Data are shown as mean±s.e.m. in the graph. Wild type mice are designated as COL4A3+/+ in the figure.

FIG. 66A-FIG. 66C phospho-smad1/5 staining in COL4A3 deficient mice. FIG. 66A, FIG. 66B. Frozen kidney section of indicated groups of animals were labeled by phospho-smad1/5 (p-smad1/5) antibody followed by FITC-conjugated secondary antibody. p-smad1/5 levels are analyzed by fluorescence microscopy. Arrows in panel (FIG. 66B) indicate nuclear accumulated p-smad1/5. FIG. 66C. Quantification of p-smad1/5 level. The number of p-smad1/5 per field of view (×400 magnification) was assessed by counting positively labeled cells in 5 random fields of view per slide, with 5 slides per experimental group. Data are shown as mean±s.e.m. in the graph.

FIG. 67A-FIG. 67U THR-123 reverses the course of mouse diabetic nephropathy. Streptozotocin-induced diabetic CD-1 mice are treated with BMP7 (300 μg/kg every other day, IP, from 1 to 6 month after diabetic induction) or THR-123 (5 mg/kg/day, orally, 5 to 6 month of diabetic induction). Representative histological PAS staining of kidney sections (FIG. 67A-FIG. 67E) and Masson's trichrome staining (FIG. 67F-FIG. 67J) from untreated control mice (FIG. 67A, FIG. 67F); 5 months diabetic nephropathy (DN) (FIG. 67B, FIG. 67G); 6 months DN (FIG. 67C, FIG. 67H); BMP7 treated 6 months DN (FIG. 67D, FIG. 67I); and THR-123 treated 6 months DN (FIG. 67E, FIG. 67J). Magnification ×400 (FIG. 67A-FIG. 67E) and ×100 (FIG. 67F-FIG. 67J). FIG. 67K-FIG. 67N: Morphometric analysis of glomeruli and tubulo-interstitium. Glomerular surface area (FIG. 67K), mesangial matrix (FIG. 67L), tubular atrophy (FIG. 67M) and relative interstitial volume (FIG. 67N) are analyzed. For the quantification of glomeruli 20 glomeruli in each mouse are analyzed. For the quantification of tubulo-interstitial lesions, 8 fields per kidney were analyzed in each mouse. FIG. 67O: The effect of THR-123 on blood urea nitrogen levels in diabetic mice. FIG. 67P-FIG. 67T: Immunofluorescence analysis (magnification ×200) of normal (FIG. 67Q) or diabetic (FIG. 67Q-FIG. 67T) mice kidneys treated with BMP7 (FIG. 67S) or THR-123 (FIG. 67T) for the detection of FSP1 and E-cadherin. Arrow in panel (FIG. 67Q) and (FIG. 67R) indicates FSP1 and E-cadherin double positive tubules. FIG. 67U. A quantitative analysis for the percentage of FSP1+ cells is provided (FIG. 67U). 10 fields per mice were analyzed. In all the analysis, control (n=5), diabetes 5 month (n=6), diabetes 6 month (n=10), BMP7-treated diabetes (n=4) and THR-123-treated diabetes (n=9) are analyzed. Data are expressed as mean+s.e.m. in the graph.

FIG. 68A-FIG. 68E PAS staining of the kidneys in the diabetic mice. PAS staining (×200) of control (FIG. 68A), 5 months diabetic nephropathy (DN) (FIG. 68B), 6 months DN (FIG. 68C), DN treated with BMP7 (FIG. 68D) or THR-123 (FIG. 68E) are shown. Control (n=5), diabetes 5 month (n=6), diabetes 6 month (n=10), diabetes treated with BMP-7 (300 μg/kg every other day, IP, from 1 to 6 month after diabetic induction, n=4), diabetes treated with THR-123 (5 mg/kg/day, orally, 5 to 6 month of diabetic induction, n=9) are analyzed. Representative picture are shown.

FIG. 69A-FIG. 69F THR-123 reduces the macrophage infiltration in mice with diabetic nephropathy. Mac-1 immunofluorescence study of normal (FIG. 69A) and diabetic nephropathy (DN) treated with vehicle, BMP7 or THR-123 (FIG. 69B to FIG. 69D). Magnification ×400. Macrophages were seldom present in the cortical area of normal kidneys (FIG. 69A). After 5 or 6 months of DN, macrophages were frequently seen around atrophic tubules (FIG. 69B, FIG. 69C). BMP7 (FIG. 69D) and THR-123 (FIG. 69E) significantly reduced the infiltration by macrophages. Representative picture are shown. FIG. 69F. The number of macrophages per field of view (×400 magnification) was assessed by counting positively labeled cells in 5 random fields of view per slide, with 5 slides per experimental group. Data are shown as mean±s.e.m. in the graph. Diabetic nephropathy is designated as DN in the figure.

FIG. 70A-FIG. 70E THR-123 increased phospho-smad1/5 labeling in DN. FIG. 70A-FIG. 70D. Frozen kidney sections of indicated group of animals were labeled with phospho-smad1/5 (p-smad1/5) antibody followed by FITC-conjugated secondary antibody. p-smad1/5 levels are analyzed by fluorescence microscopy. Arrows in panel (FIG. 70C) and (FIG. 70D) indicate nuclear accumulated p-smad1/5. (FIG. 70E) Quantification of p-smad1/5 level. The number of p-smad1/5 per field of view (×400 magnification) was assessed by counting positively labeled cells in 5 random fields of view per slide, with 5 slides per experimental group. Data are shown as mean+s.e.m. in the graph. Diabetic nephropathy is designated as DN in the figure.

FIG. 71A-FIG. 71R A combination of captopril and THR-123 inhibits progression of fibrosis associated with advanced diabetic nephropathy. Streptozotocin-induced diabetic CD-1 mice are treated with captopril (p.o. 50 mg/Kg/day, from 7 to 8 month after diabetes induction) or combination of captopril with THR-123 (p.o. 5 mg/kg/day, 7 to 8 month after diabetic induction). Representative histological PAS staining of kidney sections (FIG. 71A-FIG. 71D) and Masson's trichrome staining (FIG. 71E-FIG. 71H) of 7 months DN (FIG. 71A, FIG. 71E); 8 months DN (FIG. 71B, FIG. 71F); 8 months DN treated with captopril (FIG. 71C, FIG. 71G); 8 months DN treated with combination of captopril with THR-123 (FIG. 71D, FIG. 71H). Magnification ×400 (FIG. 71A-FIG. 71D) and ×100 (FIG. 71E-FIG. 71H). I-L: Morphometric analysis of glomerular surface area (FIG. 71I), mesangial matrix (FIG. 71J), tubular atrophy (FIG. 71K) and relative interstitial volume (FIG. 71L). For the quantification of glomeruli, 20 glomeruli in each mouse are analyzed. For the quantification of tubulo-interstitial lesions, 8 fields per kidney were analyzed in each mouse. FIG. 71M: The effect of THR-123 on blood urea nitrogen levels in diabetic mice. FIG. 71N-FIG. 71Q: Immunofluorescence analysis (magnification ×200) for the detection of E-cadherin/FSP1. Arrows in panel (FIG. 71N) and (FIG. 71O) indicate E-cadherin/FSP1 double positive tubules. FIG. 71R. A quantitative analysis for the percentage of E-cadherin/FSP1 double positive tubules is provided. 10 fields per kidney were analyzed. All analyze were performed by diabetic mice at 7 month (n=2), 8 month without treatment (n=3), captopril-treated 8 month diabetes (n=3) and combination therapy captopril with THR-123 in 8 month diabetes (n=4). Data are expressed as mean+s.e.m. in the graph.

FIG. 72A-FIG. 72E THR-123 and Captopril reduces the macrophage infiltration in mice with diabetic nephropathy. Mac-1 immunofluorescence study of 7 months diabetic nephropathy (DN) (FIG. 72A), 8 months DN (FIG. 72B), captopril-treated 8 months DN (FIG. 72C) and combination of captopril with THR-123-treated 8 months DN (FIG. 72D) are shown. In DN mice, tubular accumulated macrophages were significantly increased from 7 to 8 months DN. Captopril partially and combination of captopril with THR-123 completely inhibited the infiltration by macrophages. Representative picture are shown. FIG. 72E. The number of macrophages per field of view (×400 magnification) was assessed by counting positively labeled cells in 5 random fields of view per slide, with 5 slides per experimental group. Data are shown as mean+s.e.m. in the graph. Diabetic nephropathy is designated as DN in the figure.

FIG. 73A-FIG. 73D Blood sugar level and body weight in diabetic mice. Blood sugar level (FIG. 73A, FIG. 73B) and body weight (FIG. 73C, FIG. 73D) measurements. Data are shown as mean±s.e.m. in the graph.

FIG. 74A-FIG. 74D Captopril/THR-123 apoptosis in DN. FIG. 74A-FIG. 74C. Tubule cell apoptosis was analyzed in frozen kidney sections of indicated groups of animals by TUNEL staining. Arrows in panel (FIG. 74A) indicate TUNEL positive tubule cells (FIG. 74D) Quantification of TUNEL positive tubule cells. The number of TUNEL positive tubules per field of view (×200 magnification) was assessed by counting positively labeled cells in 5 random fields of view per slide, with 5 slides per experimental group. Data are shown as mean+s.e.m. in the graph. Diabetic nephropathy is designated as DN in the figure.

FIG. 75A-FIG. 75D Captopril/THR-123 increased phospho-smad1/5 labeling in DN. FIG. 75A-FIG. 75C. Frozen kidney sections of indicated groups of animals were labeled by phospho-smad1/5 (p-smad1/5) antibody followed by FITC-conjugated secondary antibody. phospho-smad1/5 levels are analyzed by fluorescence microscopy. Arrows in panel (FIG. 75C) indicate nuclear accumulated phosho-smad1/5. (FIG. 75D) Quantification of p-smad1/5 level. The number of p-smad1/5 per field of view (×400 magnification) was assessed by counting positively labeled cells in 5 random fields of view per slide, with 5 slides per experimental group. Data are shown as mean+s.e.m. in the graph. Diabetic nephropathy is designated as DN in the figure.

FIG. 76A-FIG. 76T THR-123 acts on tubule Alk3 as a receptor in kidney disease models. FIG. 76A-FIG. 76D, The effect of THR-123 on the IRI model kidney injury in Alk3^{flox/flox} and □GTCre; Alk3^{flox/flox} mice. After ischemia reperfusion injury the mice were given THR-123 orally (5 mg/kg/day) till the day of sacrifice. FIG. 76A-FIG. 76C, the representative H&E staining picture of indicated group of mice are shown. FIG. 76D. Percentage renal tubular necrosis in IRI. Ten fields in each group were analyzed. Data are shown as mean±s.e.m. in the graph. FIG. 76E-FIG. 76T, The effect of THR-123 on the NTN model kidney injury in Alk3^{flox/flox} and gGTCre; Alk3^{flox/flox} mice. Six weeks after NTN was introduced in the indicated groups of mice, mice were treated with PBS or THR-123 orally. FIG. 76E-FIG. 76H, the representative MTS staining picture of indicated group of mice are shown. FIG. 76I, Morphometric analyses from 9 weeks of NTN models in indicated groups. FIG. 76J-FIG. 76N, Macrophage accumulation analysis in the kidney of NTN model in Alk3^{flox/flox} and gGTCre; Alk3^{flox/flox} mice. FIG. 76J-FIG. 76M, Mac-1 immunofluorescence study of indicated groups of mice. FIG. 76N, The number of macrophages per field of view (×400 magnification) was assessed by counting positively labeled cells in 5 random fields of view per slide, with 5 slides per experimental group. Data are shown as mean±s.e.m. in the graph. FIG. 76O-FIG. 76S, EMT analysis, FIG. 76O-FIG. 76R, E-cadherin/FSP1 immunolabeling of kidney in NTN-induced Alk3^{flox/flox} and GTCre; Alk3^{flox/flox} mice treated with either PBS or THR-123. FIG. 76S. Percent of E-cadherin/FSP1 double positive tubule. 500 tubules per slide, 5 slides per experimental group were analyzed. Data are expressed as mean±s.e.m. in the graph. FIG. 76T, Blood urea nitrogen measurement in 6 and 9 weeks of NTN in indicated groups of mice (all n=4).

FIG. 77A-FIG. 77D THR-123 does not inhibit macrophage accumulation in IRI kidney of Alk-3 deleted mice. Ischemia reperfusion injury (IRI) was induced by clamping of the left renal pedicle for 25 minutes. After ischemia reperfusion injury the mice were given THR-123 orally (5 mg/kg/day) or PBS till the day of sacrifice. FIG. 77A-FIG. 77C. Mac-1 immunofluorescence studies in the indicated groups. FIG. 77D. The number of macrophages per field of view (×400 magnification) was assessed by counting positively labeled cells in 5 random fields of view per slide, with 5 slides per experimental group. Data are shown as mean+s.e.m. in the graph.

FIG. 78A-FIG. 78D THR-123 does not inhibit apoptosis in IRI kidney of Alk-3 deleted mice. Ischemia reperfusion injury (IRI) was induced by clamping of the left renal pedicle for 25 minutes. After ischemia reperfusion injury the mice were given THR-123 orally (5 mg/kg/day) or PBS till the day of sacrifice. FIG. 78A-FIG. 78C. Tubule cell apoptosis was analyzed in frozen kidney sections of indicated groups of animals by TUNEL staining. FIG. 78D. Quantification of TUNEL positive tubule cells. The number of TUNEL positive tubules per field of view (×200 magnification) was assessed by counting positively labeled cells in 5 random fields of view per slide, with 5 slides per experimental group. Data are shown as mean+s.e.m. in the graph.

FIG. 79A-FIG. 79E THR-123 does not inhibit apoptosis in NTN kidney of Alk-3 deleted mice. FIG. 79A-FIG. 79D. Tubule cell apoptosis was analyzed in frozen kidney sections of indicated groups of animals by TUNEL staining. Representative pictures were shown. FIG. 79D. Quantification of TUNEL positive tubule cells. The number of TUNEL positive tubules per field of view (×200 magnification) was assessed by counting positively labeled cells in 5 random fields of view per slide, with 5 slides per experimental group. Data are shown as mean+s.e.m. in the graph (FIG. 79E).

FIG. 80 depicts the geometric basis on which the quantitative analysis of RGB images of fluoromicrographs is based.

FIG. 81A-FIG. 81C shows an example of the analysis of the fluoromicrgraphs for a test compound using RGB analysis histograms from Photoshop. FIG. 81A is of cellular field treated with 100 mM D-glucose and corresponds to an E-cadherine signal of 0%; FIG. 81B is of a cellular field exposed to media only and corresponds to a signal of 100%; and FIG. 81C is of a cellular field exposed to both 100 mM D-glucose and 100 uM test peptide. Based on the analysis, a score of 60% is assigned to this image, that is the effect of the test peptide is to maintain 60% of the E-cadherine signal observed in media alone despite the presence of 100 mM D-glucose.

Without intending to limit the invention in any manner, or limit the disclosures provided by the above figures, the figures collectively demonstrate that THR-123 (SEQ ID NO: 1) acts through both Smad and p38 MAPK BMP signaling and inhibits renal inflammation, high-glucose (hyperglycemic) induced epithelial-mesenchymal transition (EMT) and renal fibrosis. The data provided in the above figures further suggests that compounds of the invention that target the BMP signaling pathways (e.g., Smad and p38 MAPK) without inducing osteogenicity could provide a novel pharmacological intervention in renal disease.

### DETAILED DESCRIPTION OF THE INVENTION

Recently, peptide agonists of the TGF-beta superfamily proteins have been described in U.S. Pat. No. 7,482,329, WO2007/035872, and WO2006/009836, each of which is incorporated herein by reference in their entireties. The instant invention is based on the discovery that a subset of these compounds are capable of inducing BMP signaling via BMP receptors, including type I and type II receptors, thereby causing an inhibition and/or reversal of TGF-β1-induced EMT and thus, fibrosis. Recently, it was found that transforming growth factor β (TGF-β), as a central mediator of fibrogenesis, induces EMT, which in turn, mediates fibrosis. It was further identified that BMP-7 reversed TGF-β-induced EMT, thereby suggesting the role of BMP-7 in counteracting fibrosis occurring via EMT. The peptides of the invention (as further described herein) were found to be effective in the inhibition and/or reversal of EMT and fibrosis relating to a variety of conditions, including diabetic nephropathy, liver cirrhosis, idiopathic pulmonary fibrosis, rheumatoid arthritis, atherosclerosis, cardiac fibrosis, systemic sclerosis, nepthritis, and scleroderma.

### DEFINITIONS AND USE OF TERMS

The present invention may be understood more readily by reference to the following detailed description of embodiments of the invention and the Examples included therein. Before the present methods and techniques are disclosed and described, it is to be understood that this invention is not limited to specific analytical or synthetic methods as such may, of course, vary. It is also to be understood that the terminology used herein is for the purpose of describing particular embodiments only and is not intended to be limiting. Unless defined otherwise, all technical and scientific terms used herein have the meaning commonly understood by one of ordinary skill in the art to which this invention belongs.

As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

“Aromatic amino acid,” as used herein, refers to a hydrophobic amino acid having a side chain containing at least one ring having a conjugated electron system (aromatic group). The aromatic group may be further substituted with substituent groups such as alkyl, alkenyl, alkynyl, hydroxyl, sulfanyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, □-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Aliphatic amino acid,” as used herein, refers to an apolar amino acid having a saturated or unsaturated straight chain, branched or cyclic hydrocarbon side chain. Examples of genetically encoded aliphatic amino acids include alanine, leucine, valine and isoleucine. Examples of non-encoded aliphatic amino acids include norleucine (Nle).

“Acidic amino acid,” as used herein, refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic amino acid,” as used herein, refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Polar amino acid,” as used herein, refers to a hydrophilic amino acid having a side chain that is uncharged at physiological pH, but which has a bond in which the pair of electrons shared in common by two atoms is held more closely by one of the atoms. Examples of genetically encoded polar amino acids include asparagine and glutamine. Examples of non-genetically encoded polar amino acids include citrulline, N-acetyl lysine and methionine sulfoxide.

As will be appreciated by those having skill in the art, the above classifications are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of a compound, as used herein, is a quantity sufficient to achieve a desired therapeutic and/or prophylactic effect, for example, an amount which results in the prevention of or a decrease in the symptoms associated with a disease that is being treated, e.g., the diseases associated with TGF-beta superfamily polypeptides listed above. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day, to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

An “isolated” or “purified” polypeptide or polypeptide or biologically-active portion thereof is substantially free of cellular material or other contaminating polypeptides from the cell or tissue source from which the tissue differentiation factor-related polypeptide is derived, or substantially free from chemical precursors or other chemicals when chemically synthesized.

The term “variant,” as used herein, refers to a compound that differs from the compound of the present invention, but retains essential properties thereof. A non-limiting example of this is a polynucleotide or polypeptide compound having conservative substitutions with respect to the reference compound, commonly known as degenerate variants. Another non-limiting example of a variant is a compound that is structurally different, but retains the same active domain of the compounds of the present invention. Variants include N-terminal or C-terminal extensions, capped amino acids, modifications of reactive amino acid side chain functional groups, e.g., branching from lysine residues, pegylation, and/or truncations of a polypeptide compound. Generally, variants are overall closely similar, and in many regions, identical to the compounds of the present invention. Accordingly, the variants may contain alterations in the coding regions, non-coding regions, or both.

As used herein, the term “local” or “locally,” as in local administration or co-administration of one or more therapeutics, refers to the delivery of a therapeutic agent to a bodily site that is proximate or nearby the site of an injury, adjacent or immediately nearby the site of an injury, at the perimeter of or in contact with an injury site, or within or inside the injured tissue or organ. Local administration generally excludes systemic administration routes.

As used herein, the term “pharmaceutically effective regimen” refers to a systematic plan for the administration of one or more therapeutic agents which includes aspects such as drug concentrations, amounts or levels, timing, and repetition, and any changes therein made during the course of the drug administration, which when administered is effective in treating fibrosis. The skilled artisan, which will generally include practicing physicians who are treating patients having a fibrotic condition, will appreciate and understand how to determine a pharmaceutically effective regimen without undue experimentation.

As used herein, the term “co-administering,” or “co-administration,” and the like refers to the act of administering two or more agents, therapeutics, compounds, therapies, or the like, at or about the same time. The order or sequence of administering the different agents of the invention, e.g., chemotherapeutics, antifibrotic therapies, or immunotherapeutic agents, may vary and is not confined to any particular sequence. Co-administering may also refer to the situation where two or more agents are administered to different regions of the body or via different delivery schemes, e.g., where a first agent is administered systemically and a second agent is administered local at the site of tissue injury or ongoing fibrosis, or where a first agent is administered locally and a second agent is administering systemically into the blood.

As used herein, the term “substantially reverse fibrosis” refers to where the fibrotic material or components under treatment in a target tissue or organ has been decreased or altogether eradicated. Substantial reversal of fibrosis preferably refers to where least about 10%, or about 25%, or about 50%, or more preferably by at least about 75%, or more preferably by about 85%, or still more preferably by about 90%, or more preferably still about by 95%, or more preferably still by 99% or more of the fibrotic components or material has been removed as compared to pre-treatment.

As used here, reference to “substantially inhibit fibrosis” refers to where the net amount or level of fibrosis at a desired target fibrotic site does not increase with time.

The term “pharmaceutically acceptable” as used herein, refers to a material, (e.g., a carrier or diluent), which does not abrogate the biological activity or properties of the compounds described herein, and is relatively nontoxic (i.e., the material is administered to an individual without causing undesirable biological effects or interacting in a deleterious manner with any of the components of the composition in which it is contained).

As used herein, the term “selectively” means tending to occur at a higher frequency in one population than in another population.

As used herein, the term “coupled,” as in reference to two or more agents being “coupled” together, refers to a covalent or otherwise stable association between the two or more agents. For example, a therapeutic peptide of the invention (BMP agonist peptide) may be coupled with a second anti-fibrotic agent via a covalent bond, a covalently tethered linker moiety, or through ionic interactions. Preferably, the one or more agents that are coupled together retain substantial their same independent functions and characteristics. For example, the therapeutic agent when coupled to another agent may retain its same activity as if it were independent.

As used herein, the term “targeting moiety” is a moiety that is capable of enhancing the ability of a therapeutic agent, or other agent of the invention (e.g., a BMP agonist peptide of the invention) to be targeted to, to bind with, or to enter, a target cell of the invention (e.g., a tissue having an injury and which is undergoing fibrosis). In certain embodiments, targeting moieties are polypeptides, carbohydrates or lipids. Optionally, targeting moieties are antibodies, antibody fragments or nanobodies. Exemplary targeting moieties include tumor targeting moieties, such as somatostatin (sst2), bombesin/GRP, luteinizing hormone-releasing hormone (LHRH), neuropeptide Y (NPY/Y1), neurotensin (NT1), vasoactive intestinal polypeptide (VIP/VPAC1) and cholecystokinin (CCK/CCK2). In certain embodiments, a targeting moiety is non-covalently associated with an agent of the invention.

As used herein, the term “regimen” refers to the various parameters that characterize how a drug or agent is administered, including, the dosage level, timing, and iterations, as well as the ratio of different drugs or agents to one another. The term “pharmaceutically effective regimen” refers to a particular regimen which provides a desired therapeutic result or effect, including substantial inhibition or reversal of EMT and/or fibrosis. The term “iterations” refer to the general concept of repeating sets of administering one or more agents. For example, a combination of drug X and drug Y may be given (co-administered at or about at the same time and in any order) to a patient on a first day at dose Z. Drugs X and Y may then be administered (co-administered at or about at the same time and in any order) again at dose Z, or another dose, on a second day. The timing between the first and second days can be 1 day or anywhere up to several days, or a week, or several weeks, or months. The iterative administrations may also occur on the same day, separated by a specified number of minutes (e.g., 10 minutes, 20 minutes, 30 minutes or more) or hours (e.g., 1 hour, 2 hours, 4 hours, 6 hours, 12 hours). An effective dosing regimen may be determinable by those of ordinary skill in the art, e.g., prescribing physician, using standard practices.

### Fibrotic Diseases

Fibrotic diseases are characterized by the activation of fibroblasts, increased production of collagen and fibronectin, and transdifferentiation into contractile myofibroblasts. This process usually occurs over many months and years, and can lead to organ dysfunction or death. Examples of fibrotic diseases include diabetic nephropathy, liver cirrhosis, idiopathic pulmonary fibrosis, rheumatoid arthritis, atherosclerosis, cardiac fibrosis and scleroderma (systemic sclerosis; SSc). Fibrotic disease represents one of the largest groups of disorders for which there is no effective therapy and thus represents a major unmet medical need. Often the only redress for patients with fibrosis is organ transplantation; since the supply of organs is insufficient to meet the demand, patients often die while waiting to receive suitable organs. Lung fibrosis alone can be a major cause of death in scleroderma lung disease, idiopathic pulmonary fibrosis, radiation- and chemotherapy-induced lung fibrosis and in conditions caused by occupational inhalation of dust particles. The lack of appropriate antifibrotic therapies arises primarily because the etiology of fibrotic disease is unknown. It is essential to appreciate how normal tissue repair is controlled and how this process goes awry in fibrotic disease.

### TGF-β and its Role in Fibrosis

Pro-fibrotic proteins such as transforming growth factor-beta (TGF-β) and connective tissue growth factor (CTGF) have been implicated to involve in fibrotic diseases. As TGF-β induces fibroblasts to synthesize and contract ECM, this cytokine has long been believed to be a central mediator of the fibrotic response (1). CTGF, discovered more than a decade ago as a protein secreted by human endothelial cells (2), is induced by TGF-β and is considered a downstream mediator of the effects of TGF-β on fibroblasts (3, 4). Similarly, TGF-β induces expression of the ED-A form of the matrix protein fibronectin (ED-A FN), a variant of fibronectin that occurs through alternative splicing of the fibronectin transcript (5). This induction of ED-A FN is required for TGF-β1-triggered enhancement of α-SMA and collagen type I expression (6). Thus TGF-β has been implicated as a “master switch” in induction of fibrosis in many tissues including lung (7) and kidney (ref). In this regard, TGF-β is upregulated in lungs of patients with IPF, or in kidneys of CKD patients and expression of active TGF-β in lungs or kidneys of rats induces a dramatic fibrotic response, whereas the inability to respond to TGF-β1 affords protection from bleomycin-induced fibrosis (8) or renal interstitial fibrosis (30).

### Epithelial-Mesenchymal Transition (EMT) and its Role in Fibrosis

EMT, a process whereby fully differentiated epithelial cells undergo transition to a mesenchymal phenotype giving rise to fibroblasts and myofibroblasts, is increasingly recognized as playing an important role in repair and scar formation following epithelial injury. The extent to which this process contributes to fibrosis following injury in the lung and other organs is a subject of active investigation. Recently, it was demonstrated that transforming growth factor (TGF)-β induces EMT in alveolar epithelial cells (AEC) in vitro and in vivo, and epithelial and mesenchymal markers have been colocalized to hyperplastic type II (AT2) cells in lung tissue from patients with idiopathic pulmonary fibrosis (IPF), suggesting that AEC may exhibit extreme plasticity and serve as a source of fibroblasts and/or myofibroblasts in lung fibrosis. TGF-β1 was first described as an inducer of EMT in normal mammary epithelial cells (9) and has since been shown to mediate EMT in vitro in a number of different epithelial cell lines, including renal proximal tubular, lens, and most recently alveolar epithelial cells (10-14).

### Reversal of TGF-β1-Induced EMT and Fibrosis

A number of interventions have been demonstrated to lead to the reversal of EMT. BMP-7 (bone morphogenetic protein-7) reversed TGF-β1-induced EMT in adult tubular epithelial cells by directly counteracting TGF-β-induced Smad3-dependent EMT, and evidence for reversal of renal fibrosis occurring via EMT has been shown in vivo (20). BMP-7 was able to delay EMT in lens epithelium in association with downregulation of Smad2, whereas overexpression of inhibitory Smad7 prevented EMT and decreased nuclear translocation of Smads2 and -3 (21). EMT is ameliorated in Smad3 knockout mice (15, 16), and Smad7, an antagonist of TGF-β signaling, or bone morphogenetic protein-7 (BMP-7) acting in a Smad-dependent manner, can reverse or delay fibrosis in renal and lens epithelia (21, 22). Furthermore, HGF blocks EMT in human kidney epithelial cells by upregulation of the Smad transcriptional co-repressor SnoN, which leads to formation of a transcriptionally inactive SnoN/Smad complex, thereby blocking the effects of TGF-β1 (23). These studies suggest the feasibility of modulating Smad activity as a strategy for counteracting actions of TGF-β to induce EMT. Knowledge of the precise molecular mechanisms mediating TGF-β-induced EMT and its interactions with other signaling pathways will be important for developing strategies to inhibit/reverse EMT without disrupting the beneficial effects of TGF-β signaling.

### Bone Morphogenetic Proteins (BMPs)

Bone morphogenetic proteins (BMPs) are members of the transforming growth factor beta (TGF-β) superfamily, which control cell proliferation, differentiation, migration and survival. BMPs act through two different types of serine/threonine kinase receptors, known as type I and type II. Type II receptors upon occupancy by BMP undergo phosphorylation and then phosphorylate type I receptors, also called ALKs. Phosphorylated type I receptors in turn mediate specific intracellular signaling pathways and therefore determine the specificity of the downstream signaling. Three type I receptors have been identified, ALK2, ALK3 (BMPR-IA) and ALK6 (BMPR-IB) that are structurally similar. Importantly both type I and type II receptors form homomeric and heteromeric complexes. BMP-stimulation of the target cells leads to a rearrangement of receptor complexes at the cell surface, which influence the activation of two downstream BMP signaling pathways, canonical Smad-dependent pathway (Smad 1/5/8 pathway) and non-canonical Smad-independent signaling pathway (e.g. p38 mitogen-activated protein kinase pathway, MAPK). Smad1/5/8 pathway is shown to promote kidney repair after obstruction induced renal injury (Manson S R, Niederhoff R A, Hruska K A, Austin P F., J Urol. 85:2523-30, 2011). In contrast evidence suggests p38 MAPK pathway plays an important role in promoting renal damage associated with diabetes and ischemia/reperfusion (Evans J et al. (2002) Endocrin Rev 5:599-622, 2002; Furuichi K et al. Nephrol Dial Transplant 17:399-407, 2002). As such, compounds which induce Smad 1/5/8 signaling and inhibit p38 MAPK phosphorylation are compelling anti-inflammatory and anti-fibrotic targets with broad therapeutic potential.

BMPs are also extracellular morphogenetic signaling proteins that play important roles in embryogenesis and bone formation. During embryogenesis, BMPs stimulate epithelial-mesenchymal transformation (EMT), which is essential for mesoderm and neural tube formation. However, EMT, which is characterized by the loss of cell adhesion and increased cell mobility is also stimulated in oncogenesis and metastasis, and BMP signaling has also been shown to increase cell motility and invasiveness in certain types of cancer cells (Langenfeld, et al., Oncogene 25: 685-692, 2006; Kang, et al., Exp. Cell Res. 316: 24-37, 2010). There are currently over twenty known BMPs and several of these proteins have been shown to be associated with tumor growth and metastasis from primary tumors, in particular breast and prostate tumors that metastasize to bone (Alarmo and Kallioniemi, Endocrine-Related Cancer 17: R123-R139, 2010; Dai, et al., Cancer Res. 65: 8274-8285, 2005).

As noted above, BMPs bind to membrane-bound, high affinity type I and type II serine/threonine kinase receptors, initiating a signaling cascade through the Smad pathway and other intracellular effectors that stimulates morphogenetic cell functions such as cell proliferation, cell growth, differentiation, osteogenesis, neurogenesis, and embryogenesis (Walsh et al., Trends in Cell Biology 20: 244-256, 2010). As morphogens, BMPs have proven useful in regenerative medicine, particularly in stimulating bone formation and healing bone fractures (Rider and Mulloy, Biochem. J. 429: 1-12, 2010). Cellular BMP activity is highly regulated by a number of biological BMP antagonists that bind to BMPs and prevent BMP receptor activation thereby preventing BMP-initiated signaling (Rider and Mulloy, Biochem. J. 429: 1-12, 2010). Altering the expression or activity of these BMP-antagonists can contribute to the progression of human diseases such as fibrosis and cancer (Walsh et al., Trends in Cell Biology 20: 244-256, 2010).

In addition to these BMP-binding antagonists, BMP receptor antagonists have recently been described, for example the small molecule inhibitor dosomorphin and dosomorphin derivatives. It has been suggested that BMP receptor antagonists may prove useful in clinical disorders induced by mutations in BMP receptors and signaling pathways, such as cancer, skeletal diseases, and vascular diseases (Miyazono, et al., J. Biochem. 147: 35-51, 2010).

In one aspect of the present invention, a subclass of previously disclosed peptides have been discovered to be BMP agonists, useful and effective in triggering or inducing BMP signaling, which in the context of fibrosis, results in an inhibition and/or reversal of EMT, and consequently, fibrosis in any fibrotic condition that results at least in part from EMT.

### BMP Agonist Peptides

In one aspect, the present invention provides peptides and pharmaceutical compositions comprising these peptides for the used inhibiting the EMT process, for inhibiting fibrosis and for treating diseases and disorders associated with the EMT process and/or fibrosis, e.g., renal fibrosis.

Variants, analogs, homologs, or fragments of these peptides, such as species homologs, are also included in the present invention, as well as degenerate forms thereof. The peptides of the present invention may be capped on the N-terminus or the C-terminus or on both the N-terminus and the C-terminus. The peptides of the present invention may be pegylated, or modified, e.g., branching, at any amino acid residue containing a reactive side chain, e.g., lysine residue. The peptides of the present invention may be linear or cyclized or otherwise constrained. The tail sequence of the peptide may vary in length.

The peptides can contain natural amino acids, non-natural amino acids, D-amino acids and L-amino acids, and any combinations thereof. In certain embodiments, the compounds of the invention can include commonly encountered amino acids, which are not genetically encoded. These non-genetically encoded amino acids include, but are not limited to, β-alanine (β-Ala) and other omega-amino acids such as 3-aminopropionic acid (Dap), 2,3-diaminopropionic acid (Dpr), 4-aminobutyric acid and so forth; alpha-aminoisobutyric acid (Aib); epsilon-aminohexanoic acid (Aha); delta-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Mae); phenylglycine (Phg); cyclohexylalanine (Cha); norleucine (Nle); 2-naphthylalanine (2-Nal); 4-chlorophenylalanine (Phe(4-C1)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); beta-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH.sub.2)); N-methyl valine (MeVal); homocysteine (hCys) and homoserine (hSer).

As described herein, a large number of BMP-7 agonists have recently been disclosed in patent publications, e.g., U.S. Pat. No. 7,482,329, WO2007/035872, and WO2006/009836. As described herein, the inventors of the instant application have identified a subset of these peptides that are particularly effective for the inhibition and/or reversal of fibrosis and, therefore, the treatment of diseases and disorders caused by fibrosis.

In one embodiment, the peptide used in the methods of the invention has the general structure shown in SEQ ID NO:55:

(H)-CY[YF][DN][ND][SN]S[SNQ]V[LI]CK[RK]YRS-(OH).

In other embodiments, representative peptides provided by the present invention are summarized in Table 1.

TABLE 1

| SEQ | |
|---|---|
| ID | |
| NO | Sequence |
| 1 | (H)-CYFDDSSNVLCKKYRS-(OH) |
| 2 | (H)-DYFDDSSNVL<Dap>KKYRS-(OH) |
| 3 | (H)-<Dap>YFDDSSNVLDKKYRS-(OH) |
| 4 | (H)-CYYDNSSSVLCKR<YD>RS-(OH) |
| 5 | (H)-CYYDNSSSVLCKRYRS-(OH) |
| 6 | (HCO)-CYYDNSSSVLCKRYRS-(OH) |
| 7 | (CH3CO)-CYYDNSSSVLCKRYRS-(OH) |
| 8 | (H)-CYYDNSSSVLCKKYRS-(OH) |
| 9 | (H)-<Dap>YYDNSSSVLDKRYRS-(OH) |
| 10 | (H)-CYFNDSSQVLCKRYRS-(OH) |
| 11 | (H)-CYYDDSSSVLCKKYRS-(OH) |
| 12 | (H)-CYFDDSSQVLCKKYRS-(OH) |
| 13 | (H)-CYFDDSSSVLCKKYRS-(OH) |
| 14 | (H)-CYFNDSSNVLCKKYRS-(OH) |
| 15 | (H)-CYYDDSSNVLCKKYRS-(OH) |
| 16 | (H)-CYFDDSSQVLCKRYRS-(OH) |
| 17 | (H)-CYYDDSSQVLCKRYRS-(OH) |
| 18 | (H)-CYFDNSSQVLCKRYRS-(OH) |
| 19 | (H)-CYFDNSSSVLCKRYRS-(OH) |
| 20 | (H)-CYFDNSSSVLCKKYRS-(OH) |
| 21 | (H)-CYFDDSSNVLCKRYRS-(OH) |
| 22 | (H)-CYFDDSSSVLCKRYRS-(OH) |
| 23 | (H)-CYFDNSSNVLCKKYRS-(OH) |
| 24 | (H)-CYFDNSSNVLCKRYRS-(OH) |
| 25 | (H)-CYFDNSSQVLCKKYRS-(OH) |
| 26 | (H)-CYFNDSSNVLCKRYRS-(OH) |
| 27 | (H)-CYFNDSSQVLCKKYRS-(OH) |
| 28 | (H)-CYFNDSSSVLCKKYRS-(OH) |
| 29 | (H)-CYFNDSSSVLCKRYRS-(OH) |
| 30 | (H)-CYFNNSSNVLCKKYRS-(OH) |
| 31 | (H)-CYFNNSSNVLCKRYRS-(OH) |
| 32 | (H)-CYFNNSSQVLCKKYRS-(OH) |
| 33 | (H)-CYFNNSSQVLCKRYRS-(OH) |
| 34 | (H)-CYFNNSSSVLCKKYRS-(OH) |
| 35 | (H)-CYFNNSSSVLCKRYRS-(OH) |
| 36 | (H)-CYYDDSSNVLCKRYRS-(OH) |
| 37 | (H)-CYYDDSSQVLCKKYRS-(OH) |
| 38 | (H)-CYYDDSSSVLCKRYRS-(OH) |
| 39 | (H)-CYYDNSSNVLCKKYRS-(OH) |
| 40 | (H)-CYYDNSSNVLCKRYRS-(OH) |
| 41 | (H)-CYYDNSSQVLCKKYRS-(OH) |
| 42 | (H)-CYYDNSSQVLCKRYRS-(OH) |
| 43 | (H)-CYYNDSSNVLCKKYRS-(OH) |
| 44 | (H)-CYYNDSSNVLCKRYRS-(OH) |
| 45 | (H)-CYYNDSSQVLCKKYRS-(OH) |
| 46 | (H)-CYYNDSSQVLCKRYRS-(OH) |
| 47 | (H)-CYYNDSSSVLCKKYRS-(OH) |
| 48 | (H)-CYYNDSSSVLCKRYRS-(OH) |
| 49 | (H)-CYYNNSSNVLCKKYRS-(OH) |
| 50 | (H)-CYYNNSSNVLCKRYRS-(OH) |
| 51 | (H)-CYYNNSSQVLCKKYRS-(OH) |
| 52 | (H)-CYYNNSSQVLCKRYRS-(OH) |
| 53 | (H)-CYYNNSSSVLCKKYRS-(OH) |
| 54 | (H)-CYYNNSSSVLCKRYRS-(OH) |
| 55 | (H)-CY[YF][DN][ND][SN]S[SNQ]V[LI]CK[RK]YRS-(OH) |
| 56 | (H)-CYY[DN][ND][SN]S[SNQ]V[LI]CK[RK]YRS-(OH) |
| 57 | (H)-CYF[DN][ND][SN]S[SNQ]V[LI]CK[RK]YRS-(OH) |
| 58 | (H)-CY[YF]N[ND][SN]S[SNQ]V[LI]CK[RK]YRS-(OH) |
| 59 | (H)-CY[YF]D[ND][SN]S[SNQ]V[LI]CK[RK]YRS-(OH) |
| 60 | (H)-CY[YF][DN]N[SN]S[SNQ]V[LI]CK[RK]YRS-(OH) |
| 61 | (H)-CY[YF][DN]D[SN]S[SNQ]V[LI]CK[RK]YRS-(OH) |
| 62 | (H)-CY[YF][DN][ND]SS[SNQ]V[LI]CK[RK]YRS-(OH) |
| 63 | (H)-CY[YF][DN][ND]NS[SNQ]V[LI]CK[RK]YRS-(OH) |
| 64 | (H)-CY[YF][DN][ND][SN]SSV[LI]CK[RK]YRS-(OH) |
| 65 | (H)-CY[YF][DN][ND][SN]SNV[LI]CK[RK]YRS-(OH) |
| 66 | (H)-CY[YF][DN][ND][SN]SQV[LI]CK[RK]YRS-(OH) |
| 67 | (H)-CY[YF][DN][ND][SN]S[SNQ]VLCK[RK]YRS-(OH) |
| 68 | (H)-CY[YF][DN][ND][SN]S[SNQ]VICK[RK]YRS-(OH) |
| 69 | (H)-CY[YF][DN][ND][SN]S[SNQ]V[LI]CKRYRS-(OH) |
| 70 | (H)-CY[YF][DN][ND][SN]S[SNQ]V[LI]CKKYRS-(OH) |
| 71 | (H)-CYFDDNSNVICKKYRS-(OH) |
| 72 | (H)-CYFDDNSNVLCKKYRS-(OH) |
| 73 | (H)-CYFDDNSQVICKKYRS-(OH) |
| 74 | (H)-CYFDDNSQVLCKKYRS-(OH) |
| 75 | (H)-CYFDDSSNVICKRYRS-(OH) |
| 76 | (H)-CYFDDSSNVLCKKYRS-(OH) |
| 77 | (H)-CYFDDSSQVICKKYRS-(OH) |

The following conventions have been used in referencing the sequences herein, including SEQ ID NOs: 1-77:

The standard single letter amino acid codes for the 20 naturally occurring amino acids.

Carrot brackets encompass non-natural amino acid descriptors, e.g., <Y_{D}>, which stands for D-tyrosine.

Square brackets encompass a list of choices where single letter codes are taken separately and multiple single letter code are separated by commas: e.g., [ACH,DF,RK] stands for “either Ala, Cys, His, Asp-Phe or Arg-Lys.”

Parentheses encompass atoms, e.g., (OH) stands for a hydroxyl group.

Peptide capping groups are designated by a hyphen at the beginning and end of the sequence: e.g., (H)— designates an un-capped N-terminal amino group whereas (CH3CO)— designates an acetylated N-terminus; —(OH) designates an un-capped C-terminal hydroxyl group whereas —(NH2) designates an amidated C-terminus.

In all cases the peptides can be cyclized using disulfide bonds. Cys at position 1 is disulfide bonded to the Cys at position 11.

In another embodiment, the peptide of the invention can be:
DYFDDSSNVLX₁₁KKYRS (SEQ ID NO:2), wherein X₁₁ is Dap.

In still another embodiment, the peptide of the invention can be:
X₁YFDDSSNVLDKKYRS (SEQ ID NO:3), wherein X₁ is Dap.

In yet another embodiment, the peptide of the invention can be:
CYYDNSSSVLCKRX₁₄RS (SEQ ID NO:4), wherein X₁₄ is D-Tyr.

In one embodiment, the peptide of the invention can be:
X₁YYDNSSSVLDKRYRS (SEQ ID NO:9), wherein X₁ is Dap.

In yet another embodiment, the peptide of the invention can be:
CYX₃X₄X₅X₆SX₈VX₁₀CKX₁₃YRS (SEQ ID NO:55), wherein X₃ is Phe or Tyr;
wherein X₄ is Asp or Asn; wherein X₅ is Asp or Asn; wherein X₆ is Asn or Ser; wherein X₈ is Asn, Gln or Ser; wherein X₁₀ is Ile or Leu; wherein X₁₃ is Lys or Arg.

In still another embodiment, the peptide of the invention can be:
CYYX₄X₅X₆SX₈VX₁₀CKX₁₃YRS (SEQ ID NO:56), wherein X₄ is Asp or Asn;
wherein X₅ is Asp or Asn; wherein X₆ is Asn or Ser; wherein X₈ is Asn, Gln or Ser; wherein X₁₀ is Ile or Leu; wherein X₁₃ is Lys or Arg.

In one embodiment, the peptide of the invention can be:
CYFX₄X₅X₆SX₈VX₁₀CKX₁₃YRS (SEQ ID NO:57), wherein X₄ is Asp or Asn; wherein X₅ is Asp or Asn; wherein X₆ is Asn or Ser; wherein X₈ is Asn, Gln or Ser; wherein X₁₀ is Ile or Leu; wherein X₁₃ is Lys or Arg.

In yet another embodiment, the peptide of the invention can be:
CYX₃NX₅X₆SX₈VX₁₀CKX₁₃YRS (SEQ ID NO:58), wherein X₃ is Phe or Tyr; wherein X₅ is Asp or Asn; wherein X₆ is Asn or Ser; wherein X₈ is Asn, Gln or Ser; wherein X₁₀ is Ile or Leu; wherein X₁₃ is Lys or Arg.

In still another embodiment, the peptide of the invention can be:
CYX₃DX₅X₆SX₈VX₁₀CKX13YRS (SEQ ID NO:59), wherein X₃ is Phe or Tyr; wherein X₅ is Asp or Asn; wherein X₆ is Asn or Ser; wherein X₈ is Asn, Gln or Ser; wherein X₁₀ is Ile or Leu; wherein X₁₃ is Lys or Arg.

In another embodiment, the peptide of the invention can be:
CYX₃X₄NX₆SX₈VX₁₀CKX₁₃YRS (SEQ ID NO:60), wherein X₃ is Phe or Tyr; wherein X₄ is Asp or Asn; wherein X₆ is Asn or Ser; wherein X₈ is Asn, Gln or Ser; wherein X₁₀ is Ile or Leu; wherein X₁₃ is Lys or Arg.

In one embodiment, the peptide of the invention can be:
CYX₃X₄DX₆SX₈VX₁₀CKX₁₃YRS (SEQ ID NO:61), wherein X₃ is Phe or Tyr; wherein X₄ is Asp or Asn; wherein X₆ is Asn or Ser; wherein X₈ is Asn, Gln or Ser; wherein X₁₀ is Ile or Leu; wherein X₁₃ is Lys or Arg.

In another embodiment, the peptide of the invention can be:
CYX₃X₄X₅SSX₈VX₁₀CKX₁₃YRS (SEQ ID NO:62), wherein X₃ is Phe or Tyr; wherein X₄ is Asp or Asn; wherein X₅ is Asp or Asn; wherein X₈ is Asn, Gln or Ser; wherein X₁₀ is Ile or Leu; wherein X₁₃ is Lys or Arg.

In still another embodiment, the peptide of the invention can be:
CYX₃X₄X₅NSX₈VX₁₀CKX₁₃YRS (SEQ ID NO:63), wherein X₃ is Phe or Tyr; wherein X₄ is Asp or Asn; wherein X₅ is Asp or Asn; wherein X₈ is Asn, Gln or Ser; wherein X₁₀ is Ile or Leu; wherein X₁₃ is Lys or Arg.

In yet another embodiment, the peptide of the invention can be:
CYX₃X₄X₅X₆SSVX₁₀CKX₁₃YRS (SEQ ID NO:64), wherein X₃ is Phe or Tyr; wherein X₄ is Asp or Asn; wherein X₅ is Asp or Asn; wherein X₆ is Asn or Ser; wherein X₁₀ is Ile or Leu; wherein X₁₃ is Lys or Arg.

In one embodiment, the peptide of the invention can be:
CYX₃X₄X₅X₆SNVX₁₀CKX₁₃YRS (SEQ ID NO:65), wherein X₃ is Phe or Tyr; wherein X₄ is Asp or Asn; wherein X₅ is Asp or Asn; wherein X₆ is Asn or Ser; wherein X₁₀ is Ile or Leu; wherein X₁₃ is Lys or Arg.

In still another embodiment, the peptide of the invention can be:
CYX₃X₄X₅X₆SQVX₁₀CKX₁₃YRS (SEQ ID NO:66), wherein X₃ is Phe or Tyr; wherein X₄ is Asp or Asn; wherein X₅ is Asp or Asn; wherein X₆ is Asn or Ser; wherein X₁₀ is Ile or Leu; wherein X₁₃ is Lys or Arg.

In one embodiment, the peptide of the invention can be:
CYX₃X₄X₅X₆SX₈VLCKX₁₃YRS (SEQ ID NO:67), wherein X₃ is Phe or Tyr; wherein X₄ is Asp or Asn; wherein X₅ is Asp or Asn; wherein X₆ is Asn or Ser; wherein X₈ is Asn, Gln or Ser; wherein X₁₃ is Lys or Arg.

In still another embodiment, the peptide of the invention can be:
CYX₃X₄X₅X₆SX₈VICKX₁₃YRS (SEQ ID NO:68), wherein X₃ is Phe or Tyr; wherein X₄ is Asp or Asn; wherein X₅ is Asp or Asn; wherein X₆ is Asn or Ser; wherein X₈ is Asn, Gln or Ser; wherein X₁₃ is Lys or Arg.

In one embodiment, the peptide of the invention can be:
CYX₃X₄X₅X₆SX₈VX₁₀CKRYRS (SEQ ID NO:69), wherein X₃ is Phe or Tyr; wherein X₄ is Asp or Asn; wherein X₅ is Asp or Asn; wherein X₆ is Asn or Ser; wherein X₈ is Asn, Gln or Ser; wherein X₁₀ is Ile or Leu.

In yet another embodiment, the peptide of the invention can be:
CYX₃X₄X₅X₆SX₈VX₁₀CKKYRS (SEQ ID NO:70), wherein X₃ is Phe or Tyr; wherein X₄ is Asp or Asn; wherein X₅ is Asp or Asn; wherein X₆ is Asn or Ser; wherein X₈ is Asn, Gln or Ser; wherein X₁₀ is Ile or Leu.

In other embodiments, the peptides of the invention can include any suitable variants, analogs, homologs, or fragments of those peptides of SEQ ID NOs:1-77. Compounds of the present invention include those with homology to SEQ ID NOs:1-77, for example, preferably 50% or greater amino acid identity, more preferably 75% or greater amino acid identity, and even more preferably 90% or greater amino acid identity.

In certain other embodiments, the BMP-agonist peptides of the invention can include peptides that have a similar sequence to those peptides of SEQ ID NOs: 1-77, and which specifically may include peptides having an amino acid sequence that has at least 99% or greater sequence identity to any of SEQ ID NOs: 1-77, or at least 95% or greater sequence identity to any of SEQ ID NOs: 1-77, or at least 90% or greater sequence identity to any of SEQ ID NOs: 1-77, or at least 85% or greater sequence identity to any of SEQ ID NOs: 1-77, or at least 80% or greater sequence identity to any of SEQ ID NOs: 1-77, or at least 75% or greater sequence identity to any of SEQ ID NOs: 1-77, or at least 70% or greater sequence identity to any of SEQ ID NOs: 1-77, or at least 65% or greater sequence identity to any of SEQ ID NOs: 1-77, or at least 60% or greater sequence identity to any of SEQ ID NOs: 1-77.

In the case of polypeptide sequences which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g., in sequence, in structure, in function, and in antigenic character or other function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

The invention also provides for compounds having altered sequences including insertions such that the overall amino acid sequence is lengthened, while the compound still retains the appropriate TDF agonist or antagonist properties. Additionally, altered sequences may include random or designed internal deletions that truncate the overall amino acid sequence of the compound, however the compound still retains its BMP-agonistic functional properties. In certain embodiments, one or more amino acid residues within SEQ ID NOs:1-77 are replaced with other amino acid residues having physical and/or chemical properties similar to the residues they are replacing. Preferably, conservative amino acid substitutions are those wherein an amino acid is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the BMP-agonistic properties of the altered compound.

The amino acid residues of SEQ ID NOs:1-77, analogs or homologs of SEQ ID NOs:1-77 include genetically-encoded L-amino acids, naturally occurring non-genetically encoded L-amino acids, synthetic D-amino acids, or D-enantiomers of all of the above.

It is also contemplated that the peptides of the invention may be provided in the form of a propeptide or propolypeptide. For purposes of the invention, a propeptide or propolypeptide refers to a precursor version or variant of a peptide of the invention that is substantially inactive as compared to the mature form of the peptide (i.e., substantially lacking BMP signaling activity) that further includes a cleavable or otherwise removable portion. The precursor form of the peptides of the invention preferably do not have activity or that the activity of the peptide is subdued or otherwise reduced. Such precursor forms can include cleavable moieties or extended amino acid sequences, e.g., a leader sequence or a terminal polypeptide sequence, that may be useful for a variety of reasons, for example, in cell secretion during cellular production of a peptide of the invention, or for masking the activity of a peptide of the invention until the propeptide or propolypeptide encounters the target injury site of action. For example, the propeptide or propolypeptide may contain a cleavable moiety to remove a masking portion or leader portion which is removable only within the diseased tissue due to a heightened activity (e.g. protease or enzyme) that is characteristic only of the diseased state and not present in a healthy tissue. Such masks and leader sequences are known in the art. In this way, the peptides of the invention can be “targeted” with increased specificity for the desired site of treatment.

The peptides of the present invention may be pegylated, or modified, e.g., branching, at any amino acid residue containing a reactive side chain, e.g., lysine residue, or chemically reactive group on the linker. The peptides of the present invention may be linear or cyclized. The tail sequence of the peptides may vary in length.

The peptides can contain natural amino acids, non-natural amino acids, D-amino acids and L-amino acids, and any combinations thereof. In certain embodiments, the compounds of the invention can include commonly encountered amino acids which are not genetically encoded. These non-genetically encoded amino acids include, but are not limited to, beta-alanine (beta-Ala) and other omega-amino acids such as 3-aminopropionic acid (Dap), 2,3-diaminopropionic acid (Dpr), 4-aminobutyric acid and so forth; alpha-aminoisobutyric acid (Aib); epsilon-aminohexanoic acid (Aha); delta-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); beta-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH2)); N-methyl valine (MeVal); homocysteine (hCys) and homoserine (hSer). Non-naturally occurring variants of the compounds may be produced by mutagenesis techniques or by direct synthesis.

### Nucleic Acid Molecules Encoding the Peptides of the Invention

In another aspect, the present invention also includes one or more polynucleotides or nucleic acid molecules encoding SEQ ID NOs:1-77, including degenerate variants thereof, or any BMP agonist peptide encompassed or contemplated herein.

In another embodiment, the isolated nucleic acid molecules of the invention comprise a nucleotide sequence that encodes those peptides of SEQ ID NOs: 1-77 of Table 1 or any peptide or propeptide in the scope of the invention other than those particular embodiments of Table 1. In yet another embodiment, the isolated nucleic acid molecules can be a DNA expression or cloning vector, and the vector may optionally include a promoter sequence that can be operably linked to the nucleic acid, where the promoter causes expression of the nucleotide sequence encoding the peptide or propeptides of the invention. In still another embodiment, the vector can be transformed into a cell, such as a prokaryotic or eukaryotic cell, preferably a mammalian cell, or more preferably a human cell. In even another embodiment, the vector can be a viral vector capable of infecting a mammalian cell and causing expression of a polypeptide of SEQ ID NOs: 1-77 in an animal infected with the virus. In still other embodiments, the nucleic acid molecule comprises any suitable and/or advantageous elements for expression in a host cell, whether said host cell is a prokaryotic or eukaryotic host cell and whether the expression is carried out in vitro or in vivo. In yet further embodiments, the nucleic acid molecule of the invention may comprise a somatic gene transfer vector for introducing a nucleic acid sequence that encodes a peptide of the invention, or any variant, analog, homolog, or fragment thereof, including any useful propeptide thereof, for administering to a subject in need thereof a peptide of the invention by somatic gene transfer.

For recombinant expression of one or more the polypeptides of the invention, the nucleic acid containing all or a portion of the nucleotide sequence encoding the polypeptide is inserted into an appropriate cloning vector, or an expression vector (i.e., a vector that contains the necessary elements for the transcription and translation of the inserted polypeptide coding sequence) by recombinant DNA techniques well known in the art and as detailed below.

In general, expression vectors useful in recombinant DNA techniques are often in the form of plasmids. In the present specification, “plasmid” and “vector” can be used interchangeably as the plasmid is the most commonly used form of vector. However, the invention is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound.

Another aspect of the invention pertains to host cells, which contain a nucleic acid encoding a peptide described herein. The recombinant expression vectors of the invention can be designed for expression of the peptide in prokaryotic or eukaryotic cells. Suitable host cells are discussed further in Goeddel, GENE EXPRESSION TECHNOLOGY: METHODS IN ENZYMOLOGY 185, Academic Press, San Diego, Calif. (1990).

Expression of polypeptides in prokaryotes is most often carried out in Escherichia coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

### Methods of Making the Peptides of the Invention

In another aspect, the present invention relates to methods for making the peptides of the invention. Such methods, in general, can include any suitable known method in the art for conducting such tasks, including synthetic peptide chemistry, recombinant expression of the peptides of the invention using appropriate prokaryotic or eukaryotic host cells and expression systems, or recombinant expression of the peptides as a feature of somatic gene transfer, i.e., expression as part of the administration regimen at the site of treatment.

In one embodiment, a peptide can be synthesized chemically using standard peptide synthesis techniques, e.g., solid-phase or solution-phase peptide synthesis. That is, the compounds disclosed as SEQ ID NOs:1-77 may be chemically synthesized, for example, on a solid support or in solution using compositions and methods well known in the art, see, e.g., Fields, G. B. (1997) Solid-Phase Peptide Synthesis. Academic Press, San Diego.

In another embodiment, peptides are produced by recombinant DNA techniques, for example, overexpression of the compounds in bacteria, yeast, baculovirus or eukaryotic cells yields sufficient quantities of the compounds. Purification of the compounds from heterogeneous mixtures of materials, e.g., reaction mixtures or cellular lysates or other crude fractions, is accomplished by methods well known in the art, for example, ion exchange chromatography, affinity chromatography or other polypeptide purification methods. These can be facilitated by expressing the compounds described by SEQ ID NOs:1-77 as fusions to a cleavable or otherwise inert epitope or sequence. The choice of an expression system, as well as, methods of purification are well known to skilled artisans.

For recombinant expression of one or more the compounds of the invention, the nucleic acid containing all or a portion of the nucleotide sequence encoding the peptide may be inserted into an appropriate expression vector (i.e., a vector that contains the necessary elements for the transcription and translation of the inserted peptide coding sequence). In some embodiments, the regulatory elements are heterologous (i.e., not the native gene promoter). Alternately, the necessary transcriptional and translational signals may also be supplied by the native promoter for the genes and/or their flanking regions.

A variety of host-vector systems may be utilized to express the peptide coding sequence(s). These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Expression vectors or their derivatives include, e.g. human or animal viruses (e.g., vaccinia virus or adenovirus); insect viruses (e.g., baculovirus); yeast vectors; bacteriophage vectors (e.g., lambda phage); plasmid vectors and cosmid vectors.

A host cell strain may be selected that modulates the expression of inserted sequences of interest, or modifies or processes expressed peptides encoded by the sequences in the specific manner desired. In addition, expression from certain promoters may be enhanced in the presence of certain inducers in a selected host strain; thus facilitating control of the expression of a genetically-engineered compounds. Moreover, different host cells possess characteristic and specific mechanisms for the translational and post-translational processing and modification (e.g., glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an unglycosylated core peptide; whereas expression within mammalian cells ensures “native” glycosylation of a heterologous peptide.

The biological activity, of the peptides of the invention can be characterized using any conventional in vivo and in vitro assays that have been developed to measure the biological activity of the this class of peptides. Specific in vivo assays for testing the efficacy of a compound or analog in an application to repair or regenerate damaged bone, liver, kidney, or nerve tissue, periodontal tissue, including cementum and/or periodontal ligament, gastrointestinal and renal tissues, and immune-cell mediated damages tissues are disclosed in publicly available documents, which include, for example, EP 0575,555; WO93/04692; WO93/05751; WO/06399; WO94/03200; WO94/06449; and WO94/06420.

### Pharmaceutical Compositions

The peptides and/or nucleic acid molecules of the invention, and derivatives, fragments, analogs and homologs thereof, can be incorporated into pharmaceutical compositions suitable for administration. Such compositions typically comprise the nucleic acid molecule, polypeptide, or antibody with or without a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHORr™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fingi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition a compound, which delays absorption, for example, aluminum monostearate and gelatin.

Sterile injectable solutions can be prepared by incorporating the peptide in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fasidic acid derivatives. Transmucosal administration can be accomplished through the use of nasal sprays or suppositories. For transdermal administration, the active compounds are formulated into ointments, salves, gels, or creams as generally known in the art.

The compounds can also be prepared as pharmaceutical compositions in the form of suppositories (e.g., with conventional suppository bases such as cocoa butter and other glycerides) or retention enemas for rectal delivery. The compounds can be prepared for use in conditioning or treatment of ex vivo explants or implants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The present invention also contemplates pharmaceutical compositions useful for somatic gene transfer. The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

The present invention also contemplates pharmaceutical compositions and formulations for co-administering the peptides of the invention with one or more additional active agents. The one or more additional active agents can include other anti-fibrosis therapies. The one or more additional active agents can also include other therapies relating to the underlying disease or condition that results in or is involved in or relates to the fibrotic condition. For example, in certain embodiments where the fibrosis is a component of diabetic nephropathy, liver cirrhosis, idiopathic pulmonary fibrosis, rheumatoid arthritis, atherosclerosis, cardiac fibrosis, systemic sclerosis, nepthritis, and scleroderma, the additional one or more active agents can include an agent that is effective against treating other symptoms or aspects of these underlying conditions that are different from the fibrosis itself. Additional antifibrotic agents should they be used in combination can include, for example, drugs that are primarily directed at inhibiting cytokines, chemokines, specific MMPs, adhesion molecules (integrins), and inducers of angiogenesis, such as VEGF, and drugs that inhibit fibroblast proliferation and activation or which actively induce myofibroblast apoptosis, or which remove or degrade the ECM, e.g., collagenases. It should be noted, however, that currently, there are no proven effective anti-fibrotic or combination of anti-fibrotic drugs in use. Anti-inflammatory compounds alone are not effective either but can be used in combination. Steroidal anti-inflammatory compounds (e.g., prednisone) and ACE inhibitors (e.g., perindopril, captopril, enalapril) can be tested and used to treat fibrosis in combination with the peptides of the invention.

### Methods of Treatment

The invention provides for both prophylactic and therapeutic methods of treating a subject at risk of (or susceptible to) a disorder or having a disorder associated with fibrosis. The methods and peptides of the invention are effective against any fibrotic condition, no matter the etiology or which disease or disorder results in the fibrosis. In certain embodiments, the present methods and peptides are effective against fibrosis which is caused, at least in part, by EMT, whereby the methods and peptides of the invention result inhibition and/or reversal of EMT and consequently inhibition and/or reversal of fibrosis.

It is understood and herein contemplated that the disclosed methods of treating fibrosis can be combined with any other method of treating fibrosis known in the art.

It is understood and herein contemplated that the disclosed methods of treating fibrosis can treat any fibrotic condition regardless of whether the fibrosis is the result of disease, accidental exposure to radiation, accidental tissue injury, therapeutic exposure to radiation, or surgical procedures. Thus, it is understood and herein contemplated that the disclosed methods can be used to treat fibrosis wherein the cause of the fibrosis includes but is not limited to pulmonary fibrosis caused by scleroderma lung disease, idiopathic pulmonary fibrosis (IPF), Bronchiolitis Olibterans Organizing Pneumonia (BOOP), Acute Respiratory Distress Syndrome (ARDS), asbestosis, accidental radiation induced lung fibrosis, therapeutic radiation induced lung fibrosis, Rheumatoid Arthritis, Sarcoidosis, Silicosis, Tuberculosis, Hermansky Pudlak Syndrome, Bagassosis, Systemic Lupus Erythematosis, Eosinophilic granuloma, Wegener's granulomatosis, Lymphangioleiomyomatosis, Cystic Fibrosis, Nitiofurantoin exposure, Amiodarone exposure, Bleomycin exposure, cyclophosphamide exposure, or methotrexate exposure as well as myocardial infarction, injury related tissue scarring, scarring form surgery, or therapeutic radiation induced fibrosis. Thus, for example, fibrosis in the throat following radiation treatment for throat cancer can be treated with the disclosed methods.

Accordingly, in various embodiments, the present invention relates to polypeptides/peptides and methods of administering same to treat any fibrotic condition in any tissue and/or organ of the body, including, but not limited to, fibrosis associated with diabetic nephropathy, liver cirrhosis, idiopathic pulmonary fibrosis, rheumatoid arthritis, atherosclerosis, cardiac fibrosis, systemic sclerosis, nepthritis, and scleroderma.

In certain embodiments, the peptides disclosed herein can be compounds used to treat renal dysfunction, disease and injury, e.g. ureteral obstruction, acute and chronic renal failure, renal fibrosis, and diabetic nephropathy. (Klar, S., J. Nephrol. 2003 March-April; 16(2):179-85) demonstrated that BMP-7 treatment significantly decreased renal injury in a rat model of ureteral obstruction (UUO), when treatment was initiated at the time of injury. Subsequent studies suggested that BMP-7 treatment also attenuated renal fibrosis when administered after renal fibrosis had begun. Specifically, the peptides of the invention can be used to treat kidney disease, e.g., chronic kidney disease.

In certain other embodiments, the invention may be used to treat CKD. Chronic kidney disease (CKD) is a disease afflicting an estimated 13% of Americans. Regardless of disease origin, fibrosis is a final common pathway in CKD that leads to disease progression and ultimately organ failure. Chronic kidney disease is progressive, not curable, and ultimately fatal, either because of the consequences of kidney failure or due to the high level of cardiovascular mortality in the CKD patient population.

In certain other embodiments, the peptides can be used in the prophylaxis or treatment of renal fibrosis and CKD. Exogenous administration of recombinant human bone morphogenetic protein (BMP)-7 was shown to ameliorate renal glomerular and interstitial fibrosis in rodents with experimental renal diseases (Wang and Hirschberg, Am J Physiol Renal Physiol. 2003 May; 284(5):F1006-13).

In still other embodiments, the peptides of the invention can be used in the prophylaxis or treatment of chronic liver disease. Liver fibrosis is a scarring process initiated in response to chronic liver disease (CLD) caused by continuous and repeated insults to the liver. Some major causes of CLD include viral hepatitis B and C, alcoholic cirrhosis, and non-alcoholic fatty liver disease (NAFLD). The symptoms of early-stage CLD differ according to the type of underlying damage and may be clinically silent, or can include acute inflammation, weakness and jaundice. Later stages of CLD are characterized by extensive remodeling of the liver architecture and chronic organ failure.

In yet other embodiments, the peptides of the invention can be used in the prophylaxis or treatment of various lung-related fibrotic conditions and other fibrosis conditions, including idiopathic pulmonary fibrosis (IPF), systemic sclerosis, and organ transplant fibrosis. IPF is a debilitating and life-threatening lung disease characterized by a progressive scarring of the lungs that hinders oxygen uptake. The cause of IPF is not known. As scarring progresses, patients with IPF experience shortness of breath (dyspnea) and difficulty with performing routine functions, such as activities of daily living. Approximately 40,000 cases of IPF are diagnosed annually in the U.S. and Canada, where the overall prevalence is estimated to be 150,000. A similar prevalence exists for six other interstitial lung diseases and systemic sclerosis that may benefit from antifibrotic therapy. There are no FDA-approved treatments for IPF, and approximately two-thirds of patients die within five years after diagnosis. Patients are often treated with corticosteroids and immunosuppressive agents; however, none have been clinically proven to improve survival or quality of life. It is thought that stabilization or reversal of lung fibrosis could stabilize lung function and diminish the impact of this devastating disease. The present inventive peptides and methods may be used to inhibit or reverse lung fibrosis associated with IPF.

The invention may also be used to treat systemic sclerosis, which is a degenerative disorder in which excessive fibrosis occurs in multiple organ systems, including the skin, blood vessels, heart, lungs, and kidneys. There are no effective therapies for this life-threatening disease that affects more women than men (female to male ratio, 3:1). The annual incidence of systemic sclerosis is estimated to be 19 cases per million population. The present inventive peptides and methods may be used to inhibit or reverse systemic sclerosis.

The invention also may be used to treat fibrosis associated with organ transplants. In 2005, over 50,000 solid organ transplants were conducted in the US, Japan and five major European markets. The total number of transplant procedures is expected to increase to more than 67,000 by 2015. The number of patients living with functional grafts in the US alone at year-end 2005 was nearly 164,000. While remarkable progress has been made in the ability to transplant various organs, long term preservation (greater than one year) of organ function and patient survival suffers primarily because of chronic rejection. The precise manifestations of chronic rejection vary according to the transplanted organ, but all exhibit proliferation of myofibroblasts, or related cells, ultimately resulting in fibrosis that leads to loss of function. At this time, no drugs are available for treatment of the fibroproliferative lesions of progressive chronic allograft rejection.

Thus, in various aspects, the invention includes methods of administering the peptides disclosed herein for therapeutic purposes for any fibrotic condition, and in particular, those fibrotic conditions that result from or involve EMT. The modulatory method of the invention involves contacting a cell with a peptide of the present invention, thereby modulating one or more of the activities of the cell. In one embodiment, the compound stimulates one or more activities.

These modulatory methods can be performed in vitro (e.g., by culturing the cell with the peptide) or, alternatively, in vivo (e.g., by administering the peptide to a subject or by administering a somatic gene transfer vector which then expresses the peptide in the subject as means for administration of the peptide). As such, the invention provides methods of treating an individual afflicted with disease or disorder characterized fibrosis. Effective dosages and schedules for administering the compositions of the invention may be determined empirically, and making such determinations is within the skill in the art. The dosage ranges for the administration of the compositions are those large enough to produce the desired effect in which the symptoms/disorder are/is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 ug/kg to up to 100 mg/kg of body weight or more per day, depending on the factors mentioned above. Different dosing regimens may be used as appropriate.

Following administration of a disclosed composition, such as a peptide of the invention, for treating, inhibiting, or preventing an fibrosis, the efficacy of the therapeutic peptide can be assessed in various ways well known to the skilled practitioner. For instance, one of ordinary skill in the art will understand that a composition, such as an peptide, disclosed herein is efficacious in treating or inhibiting an fibrosis in a subject by observing that the composition causes an increase or increased expression in epithelial protein markers and a decrease in mesenchymal protein markers or reduces fibrosis.

The compositions that inhibit fibrosis interactions disclosed herein may be administered prophylactically to patients or subjects who are at risk for fibrosis, for example, patients preparing to undergo radiation treatment for a cancer such as throat cancer, where fibrosis from radiation damage is a possibility.

The disclosed compositions and methods can also be used for example as tools to isolate and test new drug candidates for a variety of fibrosis related diseases including but not limited to, for example, scleroderma lung disease, idiopathic pulmonary fibrosis (IPF), Bronchiolitis Olibterans Organizing Pneumonia (BOOP), Acute Respiratory Distress Syndrome (ARDS), asbestosis, accidental radiation induced lung fibrosis, therapeutic radiation induced lung fibrosis, Rheumatoid Arthritis, Sarcoidosis, Silicosis, Tuberculosis, Hermansky Pudlak Syndrome, Bagassosis, Systemic Lupus Erythematosis, Eosinophilic granuloma, Wegener's granulomatosis, Lymphangioleiomyomatosis, Cystic Fibrosis, Nitiofurantoin exposure, Amiodarone exposure, Bleomycin exposure, cyclophosphamide exposure, methotrexate exposure, myocardial infarction, injury related tissue scarring, scarring form surgery, and therapeutic radiation induced fibrosis.

Kits and/or Pharmaceutical Packages

The present invention also contemplates kits and pharmaceutical packages that are drawn to reagents or components that can be used in practicing the methods disclosed herein. The kits can include any material or combination of materials discussed herein or that would be understood to be required or beneficial in the practice of the disclosed methods. For example, the kits could include a peptide of the invention, or one or more additional active agents. In addition, a kit can include a set of instructions for using the components of the kit for its therapeutic and/or diagnostic purposes.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The following examples further demonstrate several embodiments of this invention. While the examples illustrate the invention, they are not intended to limit it.

### EXAMPLES

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Example 1

### The Effect of BMP Agonist Peptides of the Invention on Glucose-Induced EMT

Epithelial-to-mesenchymal transition (EMT) or mesothelial-to-mesenchymal transition of peritoneal mesothelial cells has been regarded as an early mechanism of fibrosis. EMT is a process whereby epithelial cell layers lose polarity and cell-cell contacts and undergo a dramatic remodeling of the cytoskeleton. Concurrent with a loss of epithelial cell adhesion and cytoskeletal components, cells undergoing EMT acquire the expression of mesenchymal components and manifest a migratory phenotype. It has been shown that high concentrations of glucose induced epithelial-to-mesenchymal transition (EMT) of HPMC, suggested by decreased expression of E-cadherin and increased expression of α-smooth muscle actin, fibronectin, and type I collagen and by increased cell migration. Activation of TGF-β signaling is sufficient to induce EMT in cultured epithelial cells. (Miettinen P J et al. J Cell Biol 127: 2021-2036, 1994). A role for EMT in tubular atrophy and appearance of myofibroblasts in renal disease was first proposed several years ago (Strutz F et al. Exp Nephrol 4: 267-270, 1996). However, evidence for TGF-β as mediator of renal tubular EMT has only recently been reported (Oldfield M D et al. J Clin Invest 108: 1853-1863, 2001; Fan J M et al. Kidney Int 56: 1455-1467, 1999). For example, advanced glycation end products (AGE) were found to induce EMT in vitro and in diabetic rats through activation of TGF-β signaling, indicating an important role for this TGF-β-induced response in progression of diabetic nephropathy (Oldfield M D et al. J Clin Invest 108: 1853-1863, 2001). On the basis of recent studies of signaling pathways activated by TGF-β to induce EMT in various types of epithelial cells, a model of this response-specific TGF-β and BMP signaling pathways is emerging.

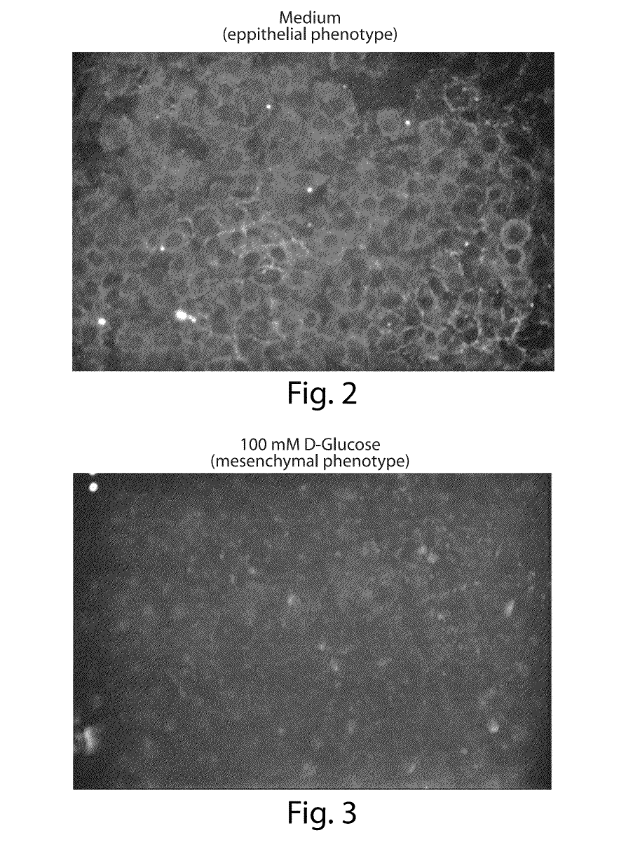

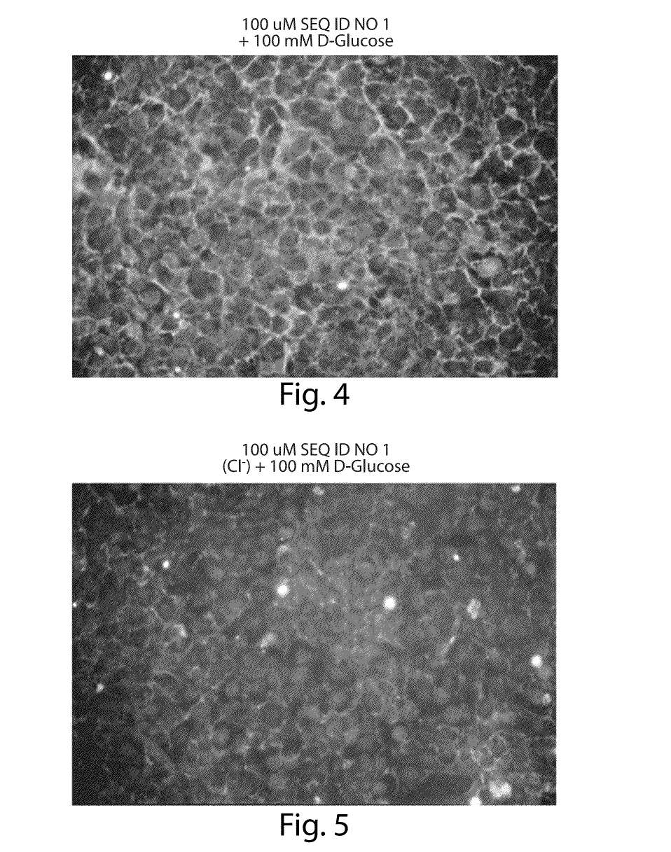

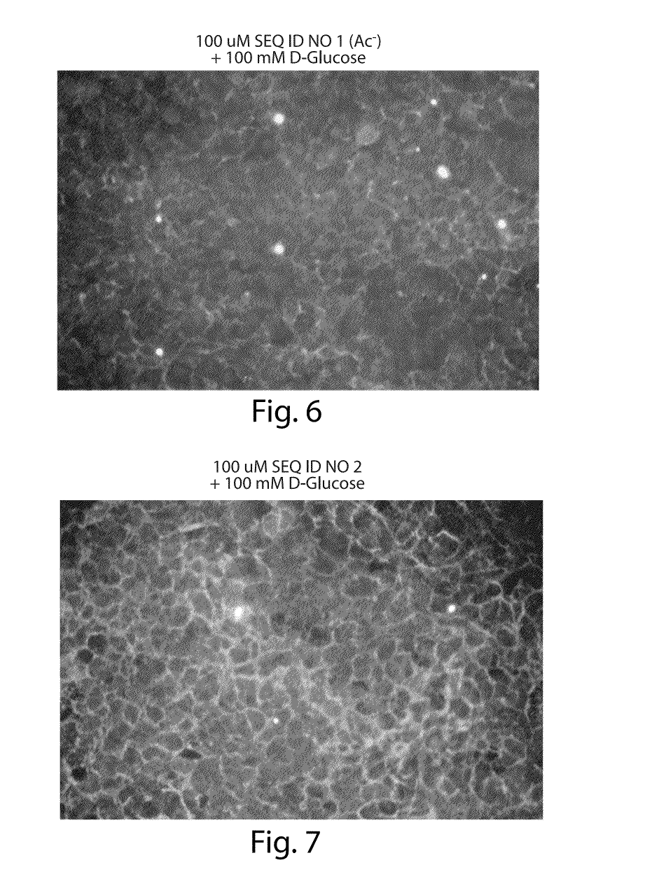

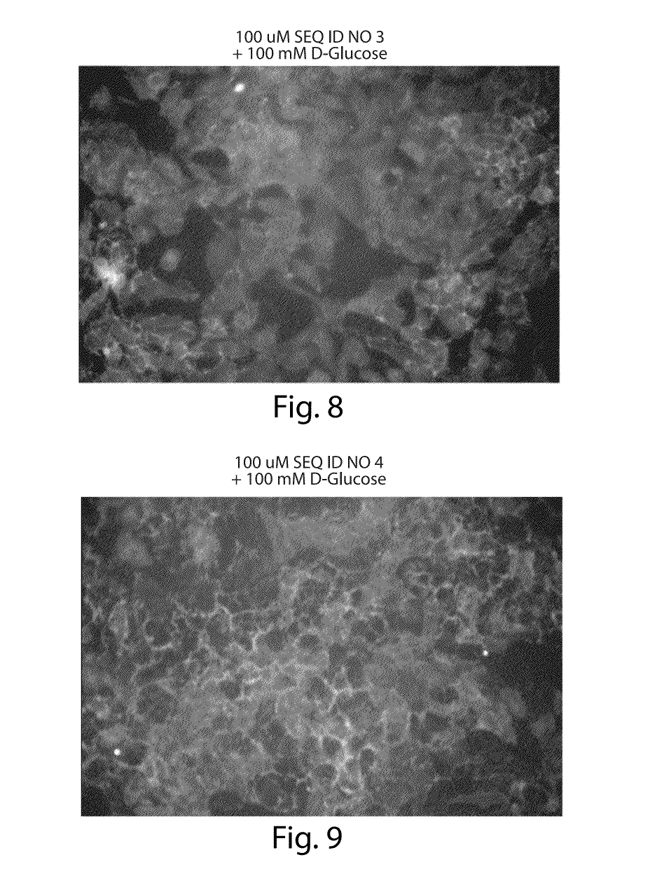

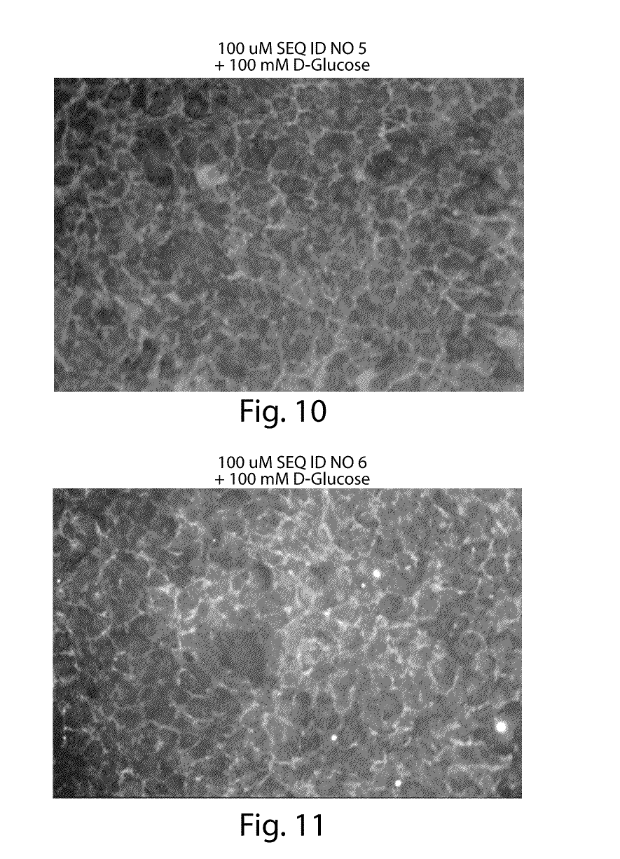

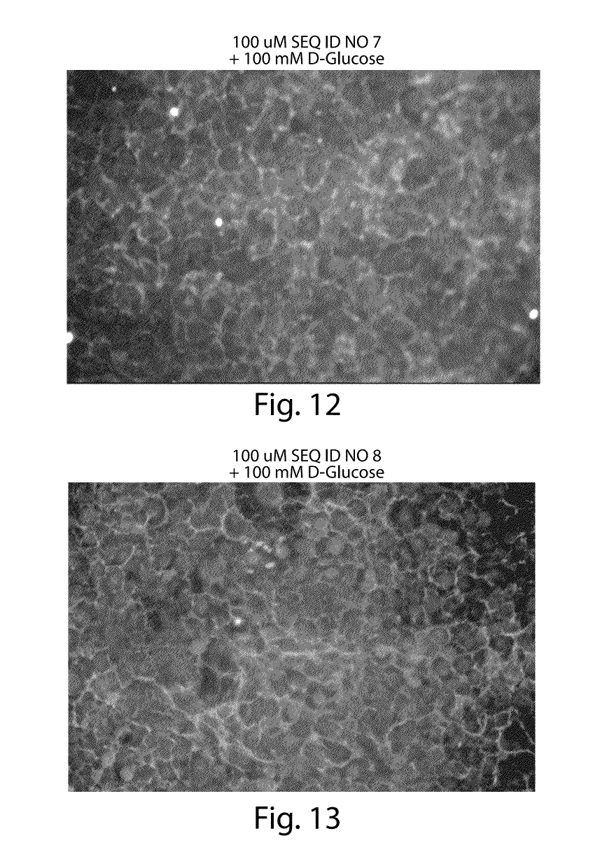

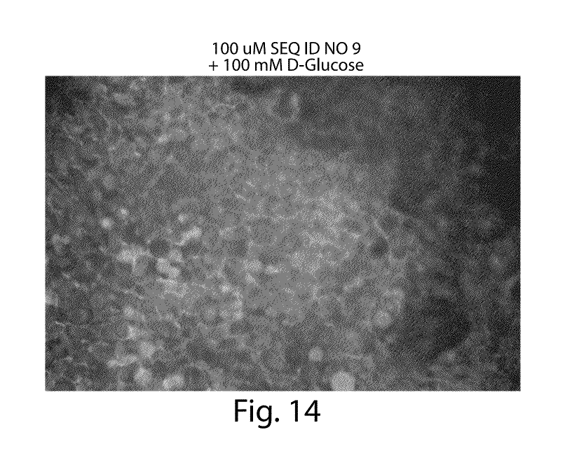

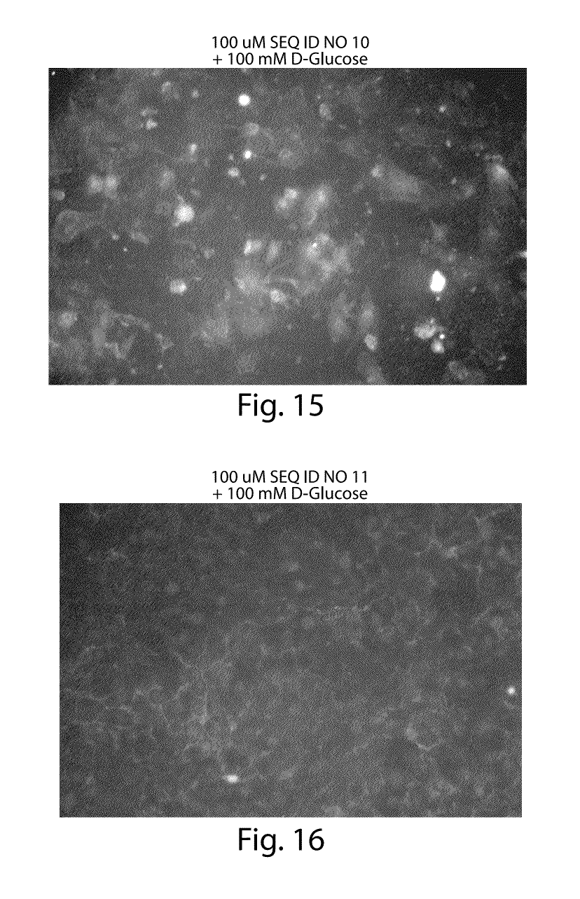

Chronic hyperglycemia is a known cause of renal fibrosis in type 2 diabetes. The assay in this example induces the transdifferentiation from the epithelial to the mesenchymal phenotype (EMT) in human proximal tubule epithelial cells (HK2) by exposing them to high levels of of D-glucose (100 mM and 200 mM). FIG. 2 shows the presence of E-cadherine (fluorescently labeled anti-E-cadherine antibody) in cells exposed to medium alone. Exposure to 100 mM D-glucose results in the loss of E-cadherine expression as evidenced by the loss of the fluorescene signal. Using this assay test compounds are evaluated based on their ability to inhibit the EMT processes in the presence of high levels of D-glucose. FIGS. 4-16 are fluorescence micrographs of HK2 cells exposed to 100 mM D-glucose and 100 uM of SEQ IS NOs 1 through 11, respectively. Note that all but SEQ ID NOs 10 and 11 were able to preserve the epithelial phenotype (i.e., inhibit the EMT).

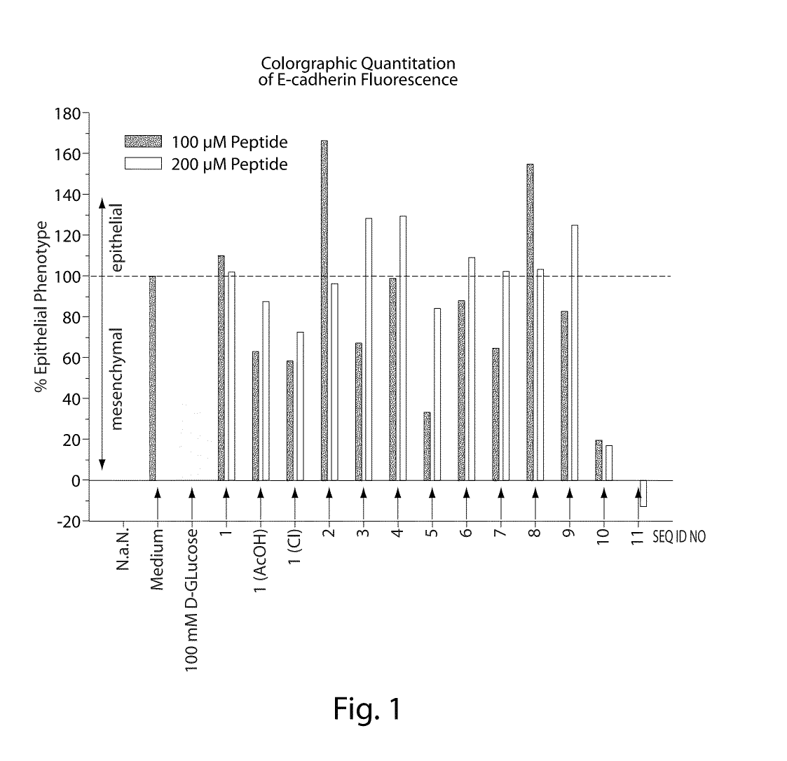

Results are provided in the context of FIG. 1 and Table 3, below. In Table 3 below, compound response is scored using image analysis as described herein in the method of Methods and Materials, part C. A 0% response corresponds to the signal for 100 mM (or 200 mM) D-glucose (untreated) while the 100% response corresponds to the signal for media in the absence of D-glucose. Peptides set forth as SEQ ID NO: 10 and SEQ ID NO: 11 had a very weak anti-fibrotic effect.

TABLE 3
Percent inhibition of D-glucose-induced EMT for
100 uM and 200 uM of a peptide of the invention

| SEQ ID NO: | 100 uM | 200 uM |
|---|---|---|
| 1 | 125% | 98% |
| 2 | 167% | 96% |
| 3 | 67% | 128% |
| 4 | 99% | 130% |
| 5 | 33% | 84% |
| 6 | 88% | 109% |
| 7 | 65% | 103% |
| 8 | 155% | 104% |
| 9 | 83% | 125% |
| 10 | 20% | 17% |
| 11 | — | −13% |

Comparing the results for SEQ ID NO: 5 to its lactam linked form (SEQ ID NO: 9) and SEQ ID NO: 1 to its lactam linked form (SEQ ID NOs: 2 & 3) suggest that the replacement of the disulfide crosslink with a lactam crosslink affect activity and may in fact increase it. Furthermore, comparing SEQ ID NO: 5 with its N-terminal capped forms (SEQ ID NOs: 6 & 7), suggest that capping the N-terminus may even increase activity. All of the compounds tested that were able to inhibit the EMT process were all also positive in anti-inflammatory assays; the fact that SEQ ID NO: 11, which is positive in anti-inflammatory assays, is unable to inhibit the EMT process implies that anti-inflammatory activity is not sufficient for anti-EMT activity.

### Example 2

### In Vivo Test of the EMT and Fibrosis Reversing Efficacy of SEQ ID NO: 1

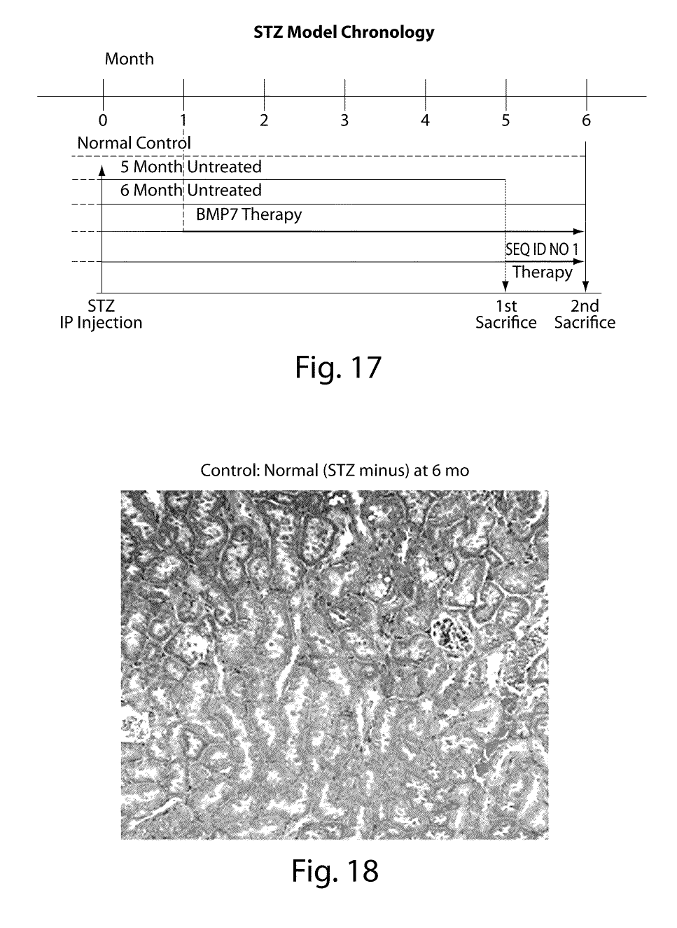

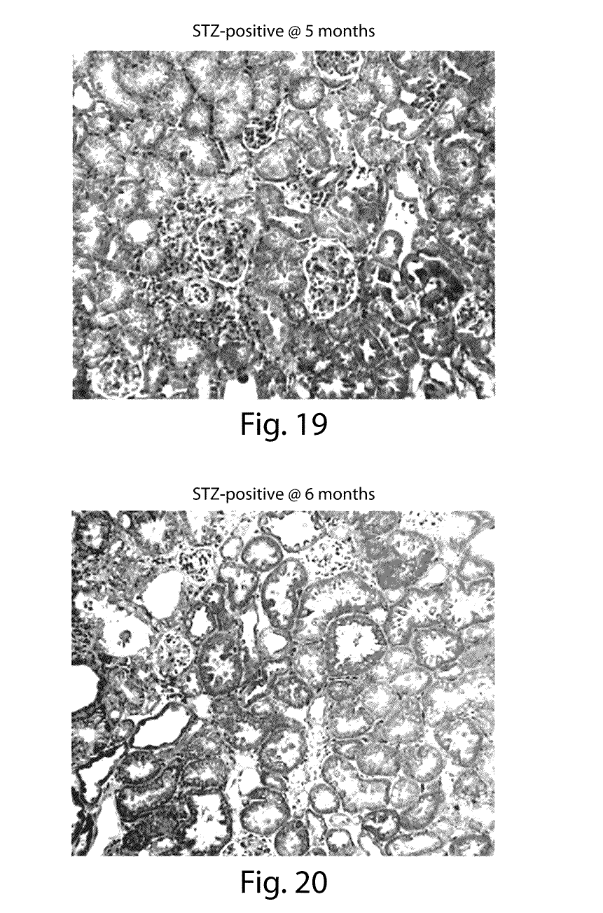

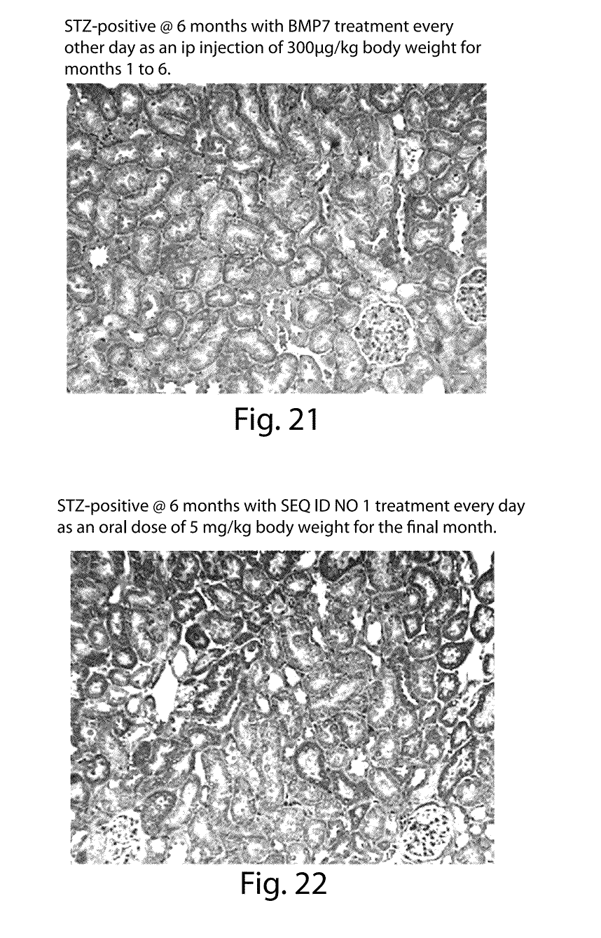

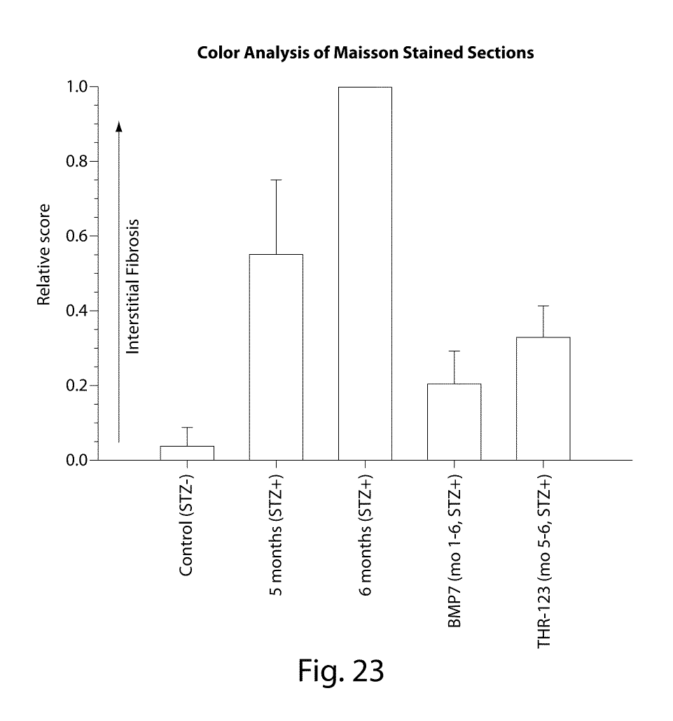

Analysis of fibrosis in the H&E and Masson's trichrome stain in FIG. 18 through 22 from the mouse STZ study outlined in FIG. 17. Using the same image color analysis method used above to quantitate E-cadherin fluorescence for the in vitro EMT experiments, the images were quantitated, and the results shown in FIG. 23.

Although the animals treated with THR-123 were treated only for the last month before sacrifice, the level of fibrotic staining tissue is lower that it would have been at 6 months, and in fact is lower than it was at 5 months, suggesting a reversal of fibrosis.

### Example 3

### Histomorphometric Analysis of Kidney Sections for all the Study Mice at 6 Months

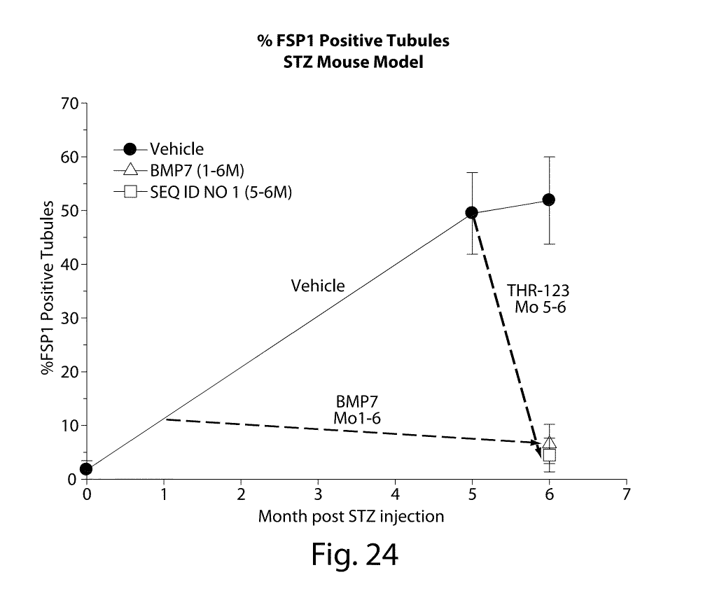

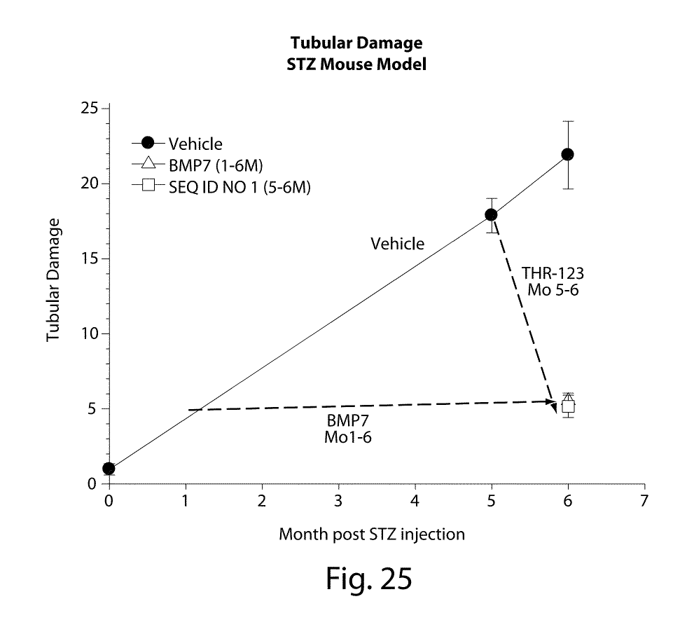

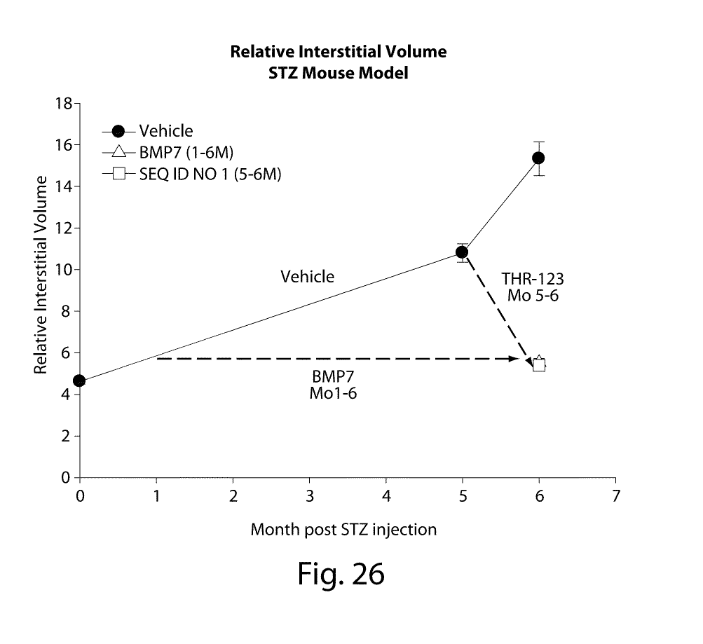

Protein FSP1 is a marker for mesenchymal tissue. As a measure of fibrosis, the presence of FSP1 was measured using fluorescent histoimmunology. As can be seen in FIG. 24, the net increase in mesenchymal tissue over a five month period was 27 times that observed for the normal animals, and over a six month period the net increase was 29 times that observed for the normal animals. These net increases in mesenchymal tissue provide strong evidence that STZ-induced diabetes caused the transformation of epithelial cells to mesenchymal cells. Treatment with BMP7 for five months reduced the net increase at six months to 3 times while treatment with SEQ ID NO: 1 for the only the final month reduced the increase to only 2 times, well below the level existing at the start of the SEQ ID NO: 1 treatment. This again provides evidence for a reversal of the EMT process. This reversal is also reflected in other morphometric parameters for tubular interstitial tissue, such as, for example, percent of damaged tubules (FIG. 25) and the increase in interstitial volume (FIG. 26).

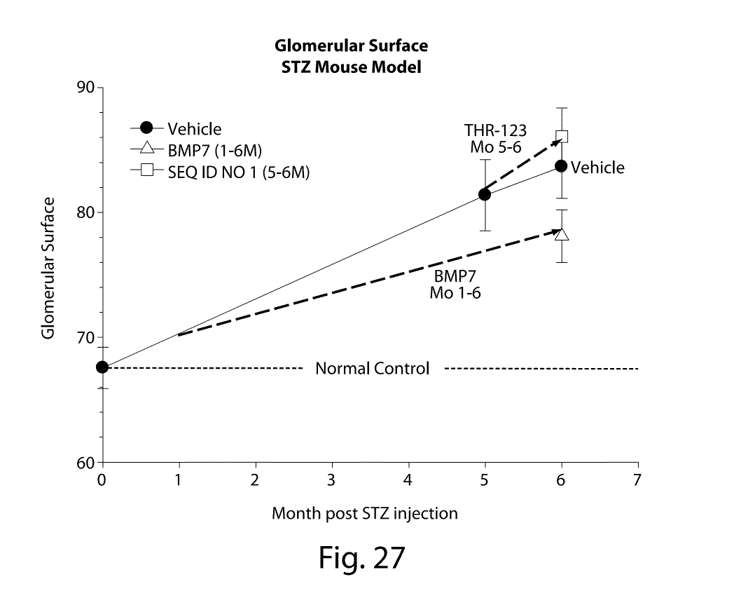

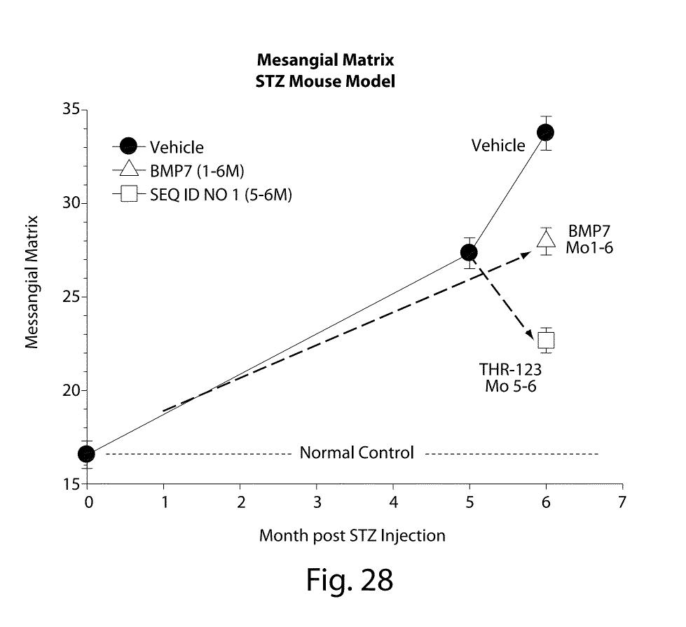

There are a few corresponding effects, however, for glomerular tissue where treatment with either BMP7 or SEQ ID NO: 1 had little effect on the 24% increase in glomerluar surface area observed over the 6 months of the study (FIG. 27). While the 5 month 64% increase and 6 month 104% increase in mesangial matrix were unaffected by BMP7 treatment, treatment with SEQ ID NO: 1 reduced the level at 6 months to 37% (FIG. 28).

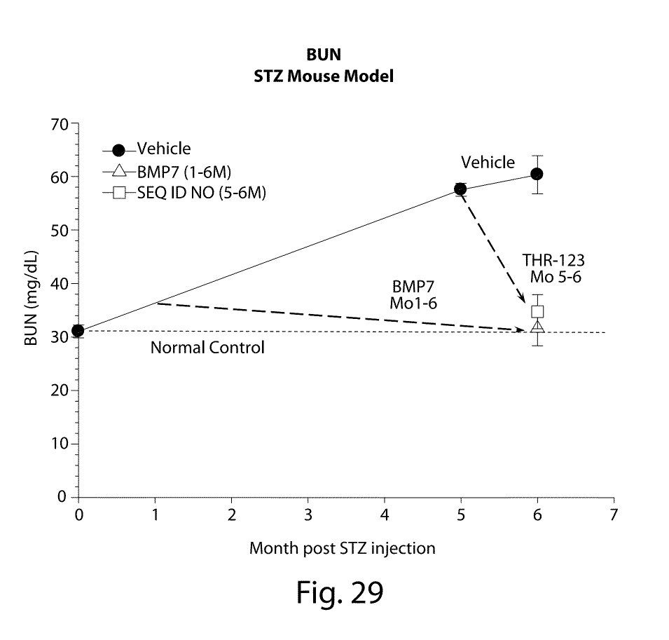

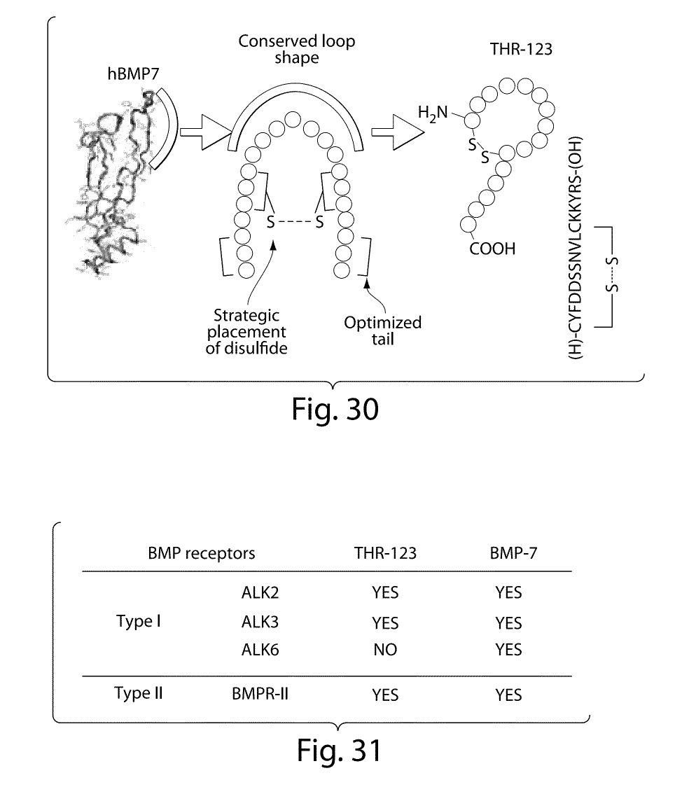

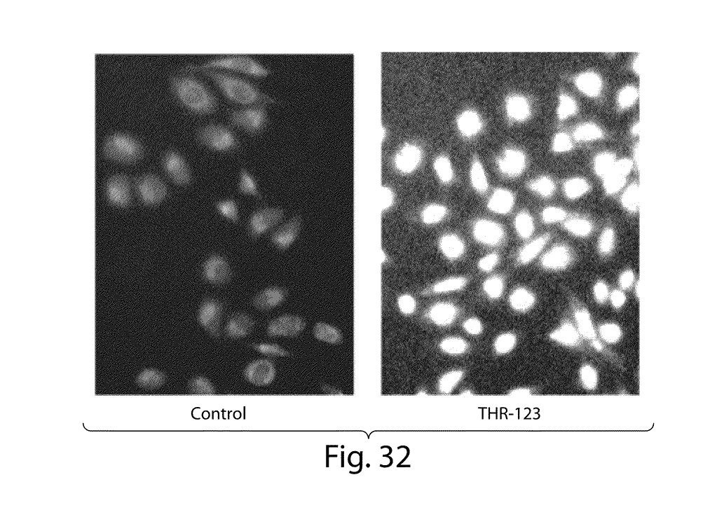

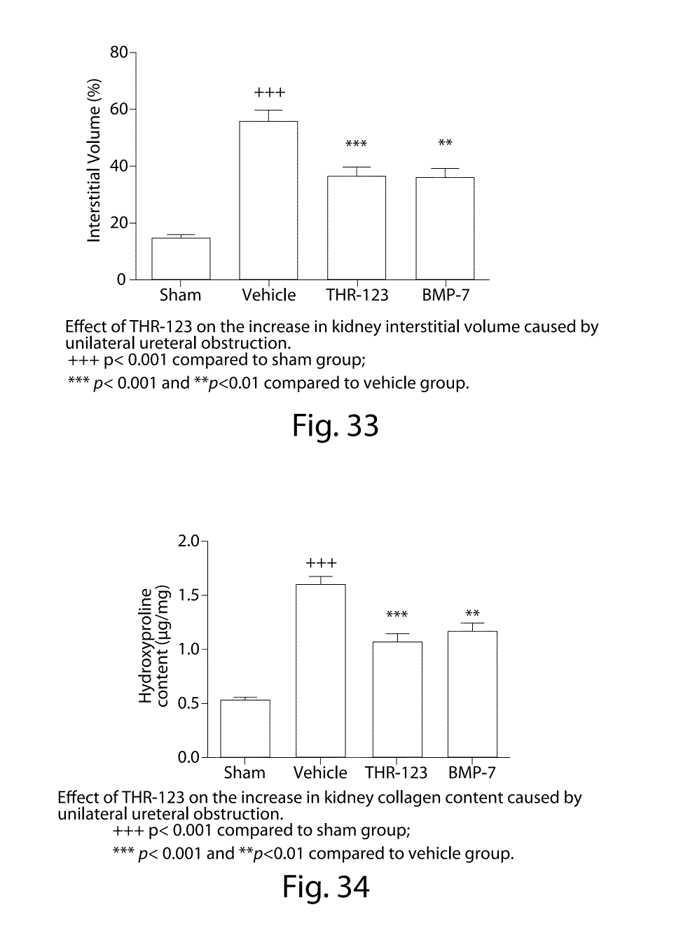

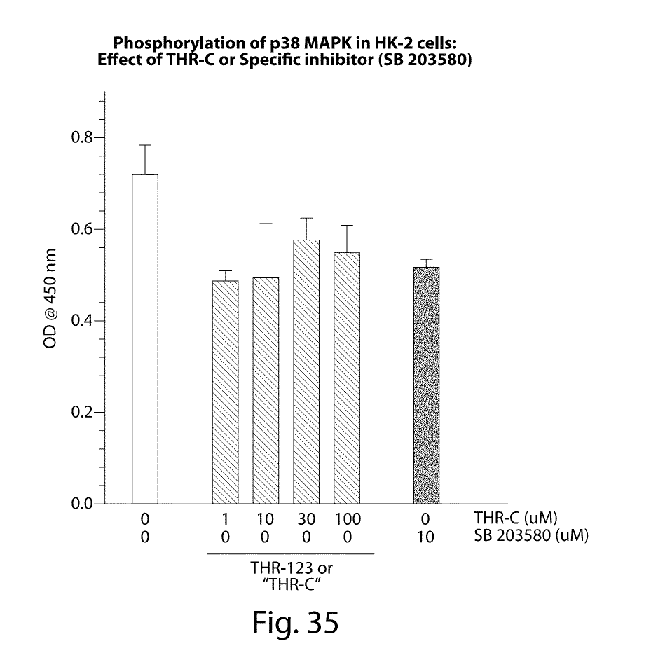

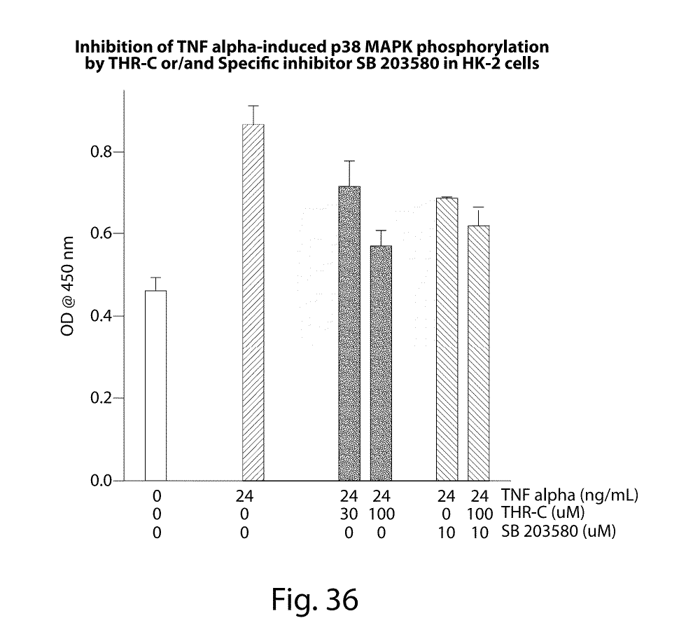

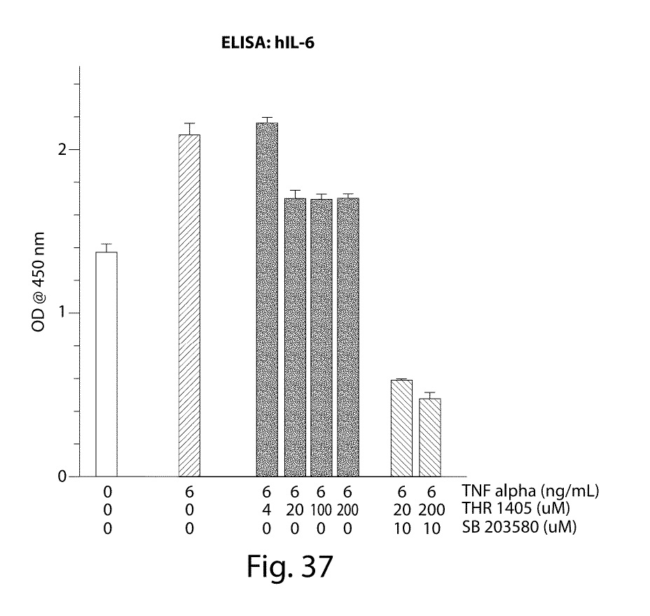

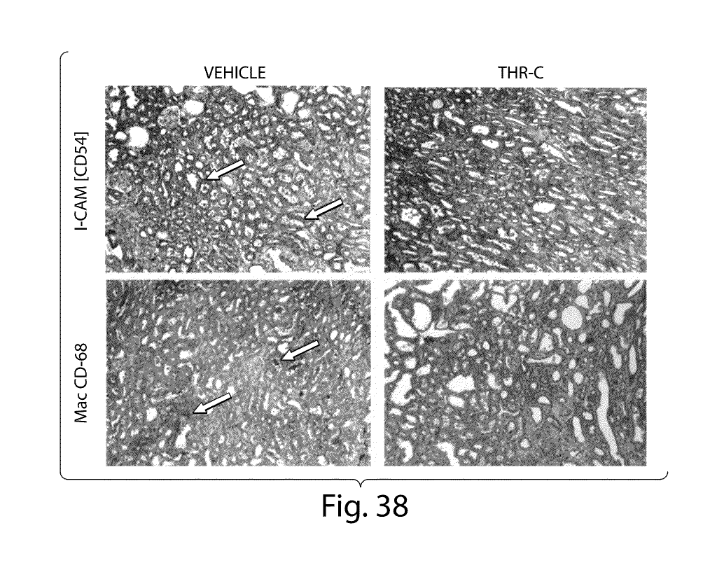

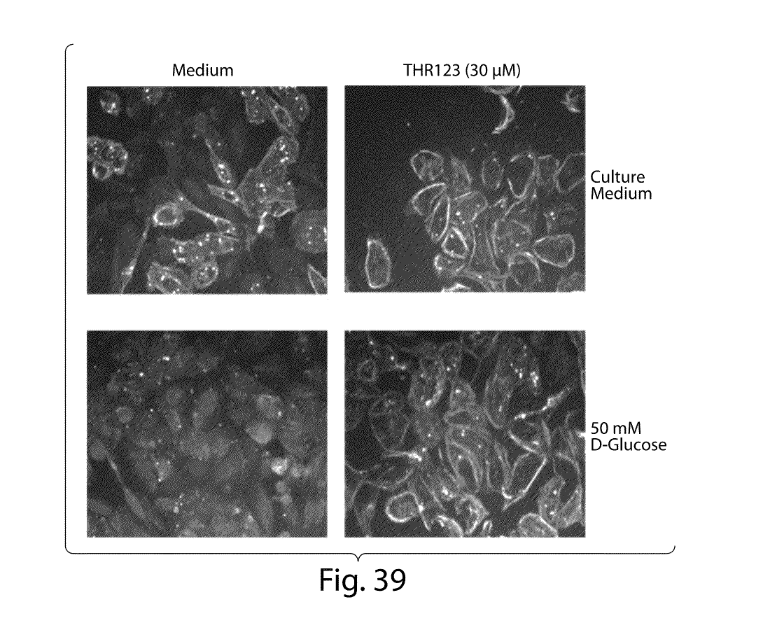

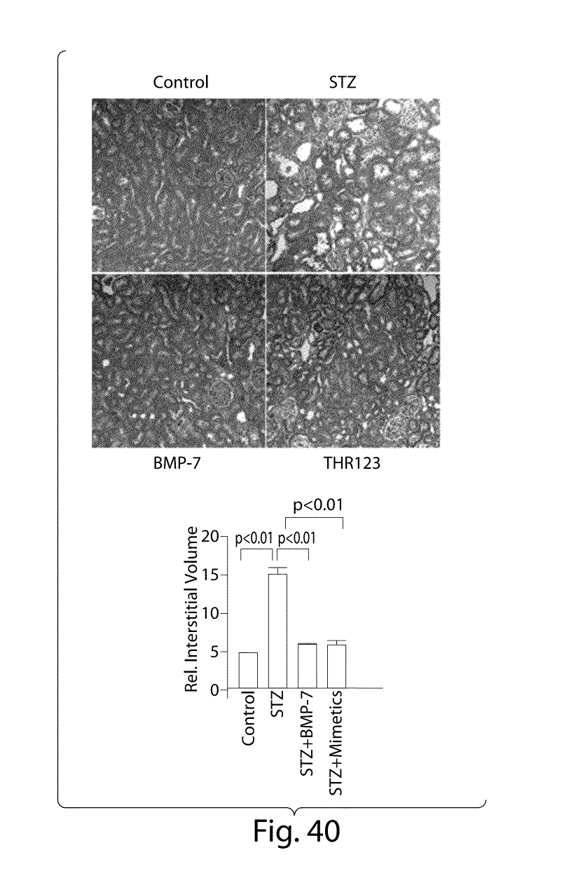

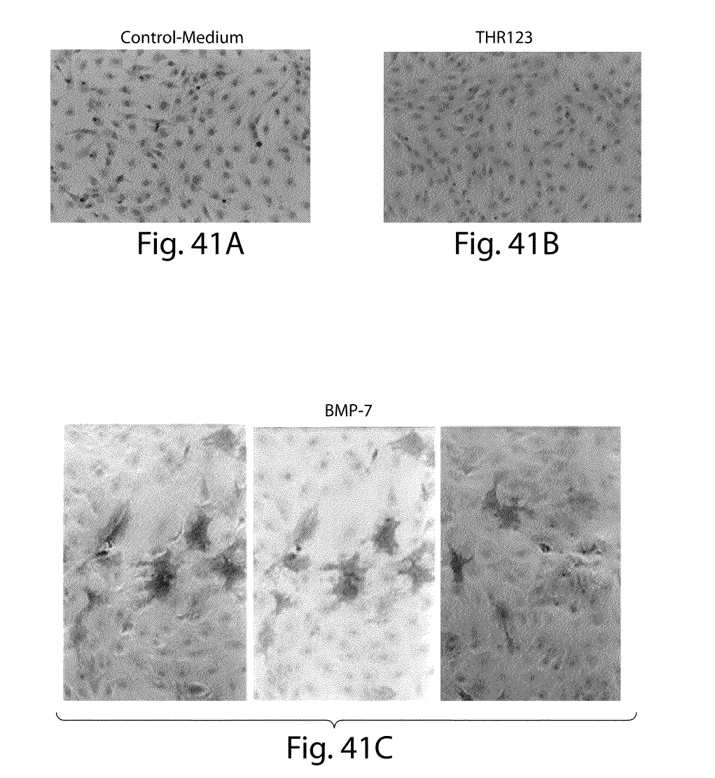

The efficacy of SEQ ID NO: 1 to reverse the effects of diabetic nephropathy is also reflected in kidney function as measured by serum blood urea nitrogen (BUN) levels. At the end of 5 months, the level increased by 85% and by 6 months it had increased by 94% relative to normal animals, which is indicative of greatly reduced renal clearance. BMP7 administered over the last 5 months kept the BUN level increase to 2%, and treatment with SEQ ID NO 1 for only the last month before sacrifice reduced the increase to 17%, well below that at the beginning of treatment (FIG. 29).

### Example 4

### Glucose-Induced EMT in Human Proximal Tubule Epithelial Cells (HK2)

It has been shown that transdifferentiation of proximal tubular epithelial cells is a critical step in the development of renal fibrosis, and that this is associated with a loss of the epithelial phenotypic marker, E-cadherin expression. This provides the basis for the development of a cell-based screening assay in which high concentrations of D-Glucose (50-100 mM) are used to induce a loss of E-cadherin expression in human renal proximal tubular epithelial (HK-2) cells, and compounds are tested for their ability to reverse the loss E-cadherin expression.

### Methods and Materials for Examples 1-4:

A. Assay Protocol

Materials:
Human proximal tubular epithelial cells, HK-2 (ATCC #CRL-2190)
Serum-free keratinocyte medium (GIBCO #17005-042, K-SFM)
Epidermal growth factor (EGF: 5 ng/mL)
Bovine pituitary extract (BPE: 40 ug/mL)
Primary antibody, Mouse IgG anti-E-cadherin (R&D Systems # MAB1838)
BSA [Sigma #A7030]
secondary antibody, FITC-conjugated Goat anti-mouse IgG (Fab₂) fragment (Sigma-Aldrich # F-2653)
Paraformaldehyde (10%))
Triton X-100 (Sigma # T-9284)
PBS (Fisher Scientific #BP399-1)
D-PBS (Invitrogen #14190-144)
Polypropylene tips (Axygen, Inc. 0.5-10 uL, cat # T-300-L-R; 1-200 uL, Cat # T-200-L-R; 1-1000 uL, Cat # T-1000-C-L-R)
50 mL polypropylene culture tubes (Fisher Scientific, cat #06-443-18)
24-well Coster cell culture plates (Fisher Scientific #07-200-84)

Reagents & Test Samples:
Positive control: BMP-7, assayed at concentrations 0.1 and 1 ug/mL
Test compounds (peptides): each tested at final concentrations 100 uM and 200 uM.

Assay Procedure for Cell Culture Assay:
Run assay in 24-well culture plates.
Seed HK-2 cells at a density between 25,000 and 30,000 cells per well in lmL K-SFM medium with supplements added (growth medium)
Incubate HK-2 cells for 24 hours at 37° C., and let cells attach the plate
Next Day afternoon, change the medium with K-SFM medium with no added supplements (serum free medium) in all wells except the first two control wells.
Continue incubating HK-2 cells overnight at 37° C.
Next Day, aspirate the medium from all wells. Incubate the cells in growth medium (first two control wells) or expose them to test compounds for 2 hours at 37° C. Final concentrations of each Test compound are 100 and 200 uM. BMP-7 serves as a positive control in the assay.
After incubation, add D-glucose to all wells except first two control wells. Final concentrations of D-Glucose are 50 and 100 mM.
Incubate HK-2 cells for 60 hours at 37° C.
Aspirate the medium from all wells. To the wells, add pre-warmed growth medium or serum free medium containing D-glucose alone or D-glucose and test compound or D-glucose and BMP-7. Final concentrations of D-Glucose and Test compound are the same as above.
Continue incubating HK-2 cells for 24 hours at 37° C.

B. Immunofluorescence Staining of HK-2 Cells for E-Cadherin Expression, and Microscopy
Aspirate the medium from all wells, and wash cells twice with PBS.
Fix cells in 3.7% paraformaldehyde in PBS for 15 minutes at room temperature.
Incubate cells with 0.2% Triton X-100 in PBS for 5 minutes
Wash cells once with PBS
Block cells with 2% BSA in PBS for 1 hour at room temperature.
Dilute primary antibody (Mouse anti-E-cadherin antibody) 1:20 using 1% BSA in PBS
Incubate cells with diluted primary antibody for 1 hour at room temperature.
Wash cells twice with PBS
Dilute second antibody (FITC labeled goat anti mouse IgG) 1:20 using 1% BSA in PBS
Incubate cells with diluted second antibody for 1 hour at room temperature
Wash cells twice with PBS
Mount cells in PBS (1 mL/well)
Visualize cells by fluorescence microscope and take pictures immediately.

C. Colorimetric Quantification of Immunofluorescent Images

Except for possibly the experienced researcher, it is difficult to judge how active a test compound is by looking at images of E-cadherin cellular fluorescence (relative to media alone). The strength of the signal is the degree to which the loss of the epithelial phenotype (i.e., E-cadherin expression) is reversed by the test treatment. While fluorescence intensity would be the obvious signal, it suffers from being an extrinsic property making it hard to measure accurately without some internal reference. On the other hand, an intrinsic property such as color does not depend on intensity. It was found that there exists a color shift (an intrinsic property) reflected in the RGB intensity histograms for regions of pixels. CRT color is composed from three luminous colors: red, green and blue (RGB). The intensity level is generally one byte wide (0 to 255), so pixel color is coded as three quantities, {R, G, B}. For various shades of grey from white {255, 255, 255} to black {0, 0, 0}, R=G=B. Violet, on the other hand, is {200, 100, 200}. This then is the basis of the image analysis of the E-cadherin fluorescence photos that were undertaken to provide a quantitative criterion for measuring in vitro anti-fibrotic activity.

D. E-Cadherin Fibrosis Assay Quantification Method

An area of fluorescence is selected on the digital photo and the RGB statistics of the pixels is computed. These three values, R, G & B, associated with each pixel can be considered to be a three dimensional color-intensity vector. The length of this vector is L=√(R²+G²+B²). The color vector, {ρ, γ, β}, is a unit vector (length=1.0), i.e, {ρ, γ, β} E {R/L, G/L, B/L}={R/L, G/L, B/L) that is then independent of intensity and so is an intrinsic property. The color vector can be thought of as a point on the surface of a unit sphere (first octant only as all components are positive). The best way to calculate the color vector for an image field is as the average vector, but instead one generally obtains from image analysis programs such as Photoshop (Adobe Systems) the statistics for each R, G and B component as a histogram using (c.f., using the image/histogram image analysis function in PhotoShop). These histograms tend to be Gaussian in shape, but have tails so it is best to use the median rather than the mean of each histogram as the component value for the overall color intensity vector representing the selected region of the image.

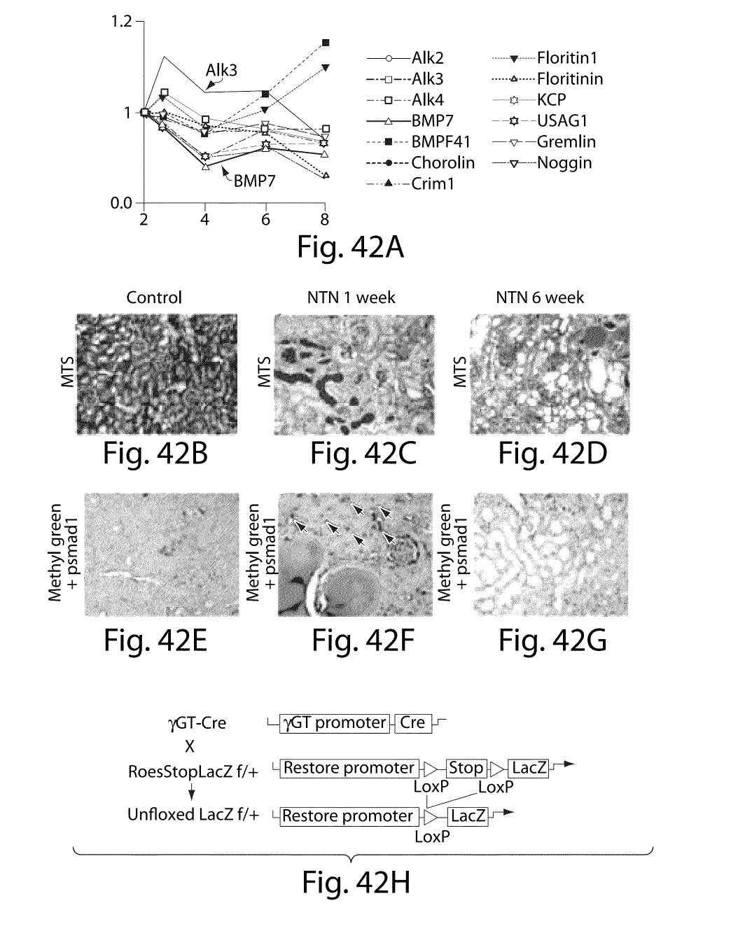

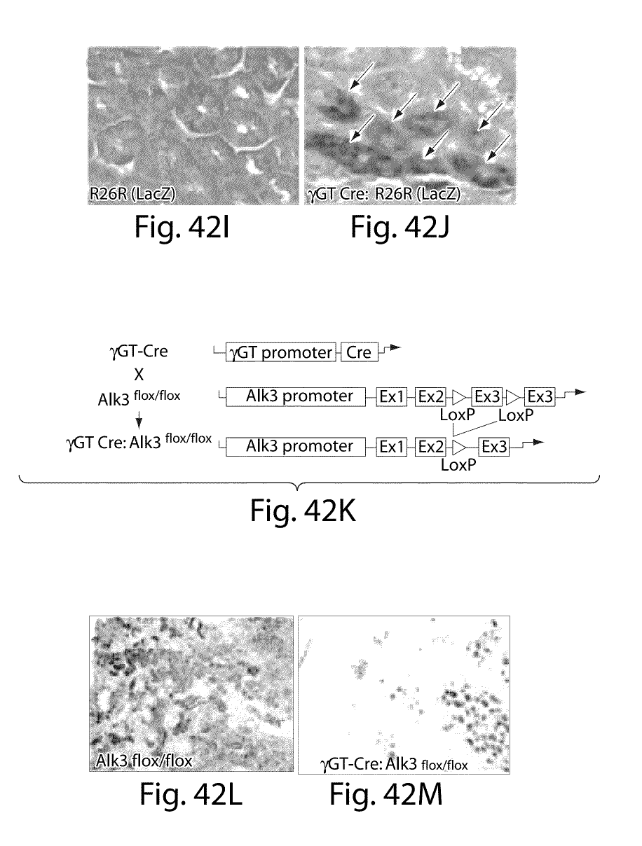

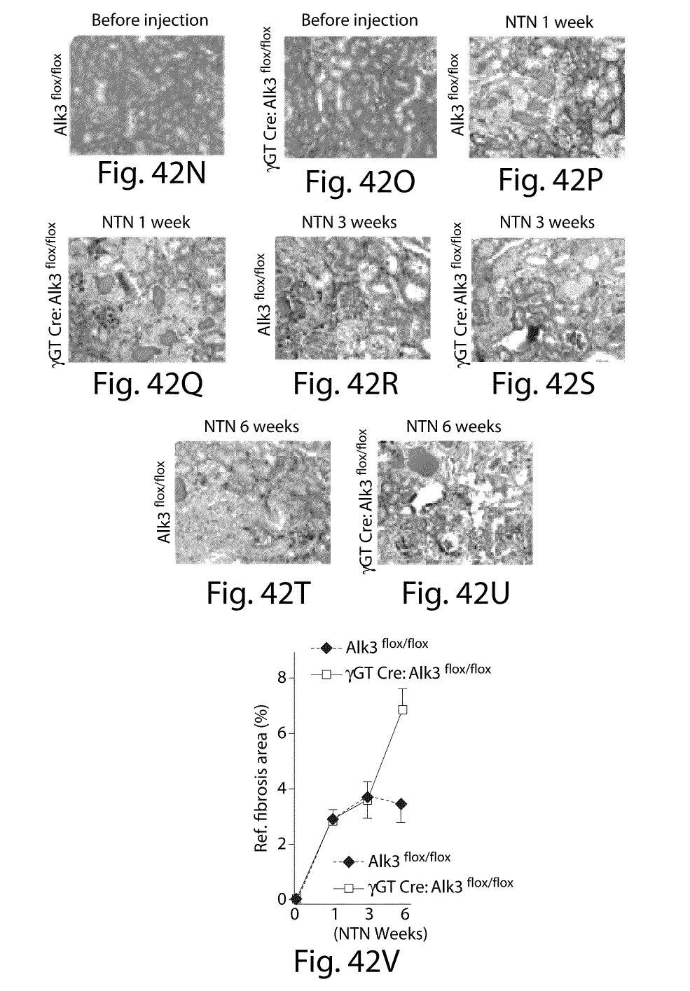

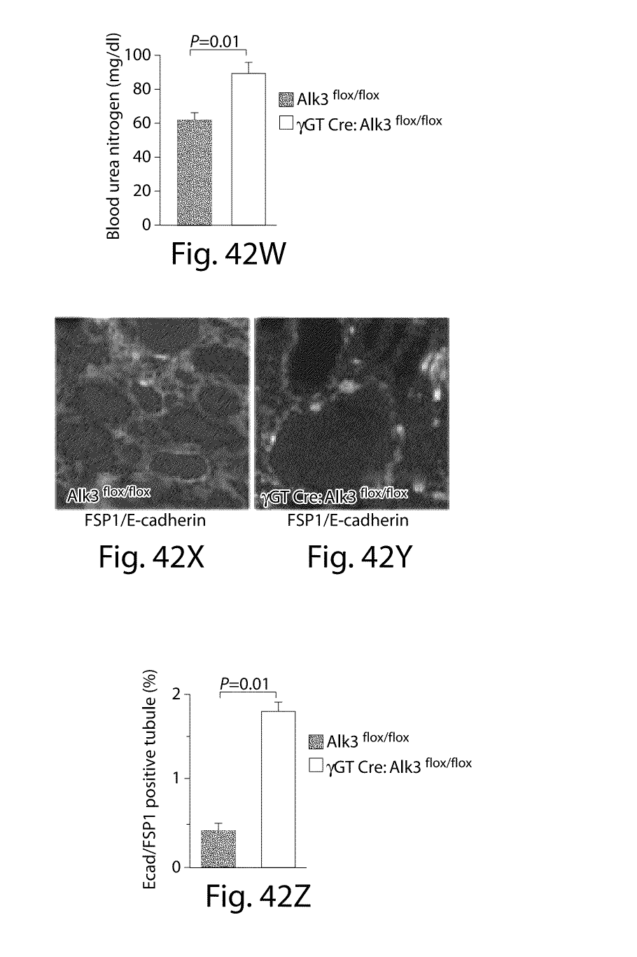

If there is a color change when fluorescent anti-bodies bind to E-cadherin on the membrane, then there will be two different color vectors, one representing media treated cells and the other representing cells treated with D-glucose. These two points define an arc of a great circle (divides the sphere in half). All data points resulting from test article treatments should fall along the arc (which we will call the signal arc) between these end points (see FIG. 42). By calling the end of the signal arc representing D-glucose treated cells zero and the other end representing normal media treated cells 1.0, the color vector representing test article treated cells can be assigned a value based on how far along the arc the color vector lies.

Vectors \vec{0} and \vec{1}, are, respectively, the color vectors for D-glucose treated (fibrotic) and untreated (media only) cells. They lie on the great circle consisting of all points perpendicular to the vector product of \vec{0} with \vec{1}, vector \hat{N}=(\vec{0}X\vec{1})/|\vec{0}X\vec{1}|. Color vector Ĉ is a data point (the color vector for a region of an image of test article treated cells), which is expected to lie near, but not necessarily exactly on, the signal arc between \hat{0} and \hat{1}. To find vector Ŝ, the component of Ĉ that lies on the signal arc, we first calculate the vector product \hat{N}XĈ, which vector defines the great circle containing both \hat{N} and Ĉ. Vector Ŝ, is one of the two intersection points of these two great circles and is given by the vector product Ŝ=(\hat{N}XĈ)X\hat{N}/|(\hat{N}XĈ)X\hat{N}|. The signal associated with color vector Ĉ is then the ratio of the angle from \hat{0} to Ŝ divided by the angle from \hat{0} to \hat{1}.

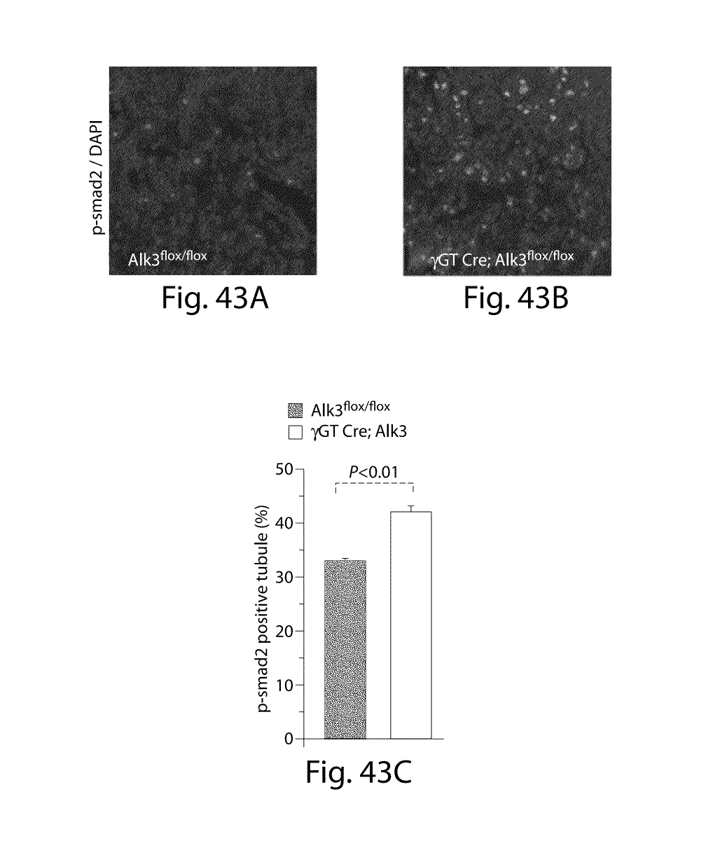

As an example, the following figures (FIG. 43) display fluorescent images of cells treated with 100 mM D-glucose, media alone and 100 mM D-Glucose plus a test compound at 100 uM. The 100 mM D-glucose image has RGB medians of (47, 94, 74), which gives a color vector {ρ, γ/β}_{0%}=(0.674, 0.531, 0.514). The media image has RGB medians of (1, 93, 31), which gives a color vector {ρ, γ, β}_{100%}=(0.010, 0.949, 0.316). The angle between these vectors is 28.4° and the normal vector defining the great circle on which they lie is \hat{N}=(−0.662, −0.231, 0.713). The experimental compound image has RGB medians of (1, 105, 75), which gives a color vector {ρ, γ, β}_{?%}=(0.008, 0.814, 0.581). The component vector Ŝ is calculated to be (0.158, 0.887, 0.434). The angle from \hat{0} to Ŝ is +17.0° so that the signal is 17.0°/28.4°=59.8%.

E. Error Detection

One should suspect color vectors that lie too far away from the signal arc. The distance off of the signal arc is given by the angle between color vectors Ĉ and Ŝ. As a measure of how far a point lies off the signal arc we take the ratio of the angle between Ĉ and Ŝ to the angle between \hat{0} and \hat{1}. A value of the ratio greater than 0.1 is a reasonable criterion for concern.

### Example 5

### Use of the Peptides of the Invention in Treating Fibrotic Disorders

Fibrotic diseases are characterized by the activation of fibroblasts, increased production of collagen and fibronectin, and transdifferentiation into contractile myofibroblasts. This process usually occurs over many months and years, and can lead to organ dysfunction or death. Examples of fibrotic diseases include diabetic nephropathy, liver cirrhosis, idiopathic pulmonary fibrosis, rheumatoid arthritis, atherosclerosis, cardiac fibrosis and scleroderma (systemic sclerosis; SSc). Fibrotic disease represents one of the largest groups of disorders for which there is no effective therapy and thus represents a major unmet medical need. Often the only redress for patients with fibrosis is organ transplantation; since the supply of organs is insufficient to meet the demand, patients often die while waiting to receive suitable organs. Lung fibrosis alone can be a major cause of death in scleroderma lung disease, idiopathic pulmonary fibrosis, radiation- and chemotherapy-induced lung fibrosis and in conditions caused by occupational inhalation of dust particles. The lack of appropriate antifibrotic therapies arises primarily because the etiology of fibrotic disease is unknown. It is essential to appreciate how normal tissue repair is controlled and how this process goes awry in fibrotic disease.

TGF Beta and its Role in Fibrosis

Pro-fibrotic proteins such as transforming growth factor-beta (TGF-β) and connective tissue growth factor (CTGF) have been implicated to involve in fibrotic diseases. As TGF-β induces fibroblasts to synthesize and contract ECM, this cytokine has long been believed to be a central mediator of the fibrotic response (1). CTGF, discovered more than a decade ago as a protein secreted by human endothelial cells (2), is induced by TGF-β and is considered a downstream mediator of the effects of TGF-β on fibroblasts (3, 4). Similarly, TGF-β induces expression of the ED-A form of the matrix protein fibronectin (ED-A FN), a variant of fibronectin that occurs through alternative splicing of the fibronectin transcript (5). This induction of ED-A FN is required for TGF-β1-triggered enhancement of α-SMA and collagen type I expression (6). Thus TGF-β has been implicated as a “master switch” in induction of fibrosis in many tissues including lung (7) and kidney (ref). In this regard, TGF-β is upregulated in lungs of patients with IPF, or in kidneys of CKD patients and expression of active TGF-β in lungs or kidneys of rats induces a dramatic fibrotic response, whereas the inability to respond to TGF-β1 affords protection from bleomycin-induced fibrosis (8) or renal interstitial fibrosis (30).

Epithelial-Mesenchymal Transition (EMT) & its Role in Fibrosis

EMT, a process whereby fully differentiated epithelial cells undergo transition to a mesenchymal phenotype giving rise to fibroblasts and myofibroblasts, is increasingly recognized as playing an important role in repair and scar formation following epithelial injury. The extent to which this process contributes to fibrosis following injury in the lung and other organs is a subject of active investigation. Recently, it was demonstrated that transforming growth factor (TGF)-β induces EMT in alveolar epithelial cells (AEC) in vitro and in vivo, and epithelial and mesenchymal markers have been colocalized to hyperplastic type II (AT2) cells in lung tissue from patients with idiopathic pulmonary fibrosis (IPF), suggesting that AEC may exhibit extreme plasticity and serve as a source of fibroblasts and/or myofibroblasts in lung fibrosis. TGF-β1 was first described as an inducer of EMT in normal mammary epithelial cells (9) and has since been shown to mediate EMT in vitro in a number of different epithelial cells, including renal proximal tubular, lens, and most recently alveolar epithelial cells (10-14).

Role for Smad-Dependent &-Independent Signaling in EMT and Fibrosis

Modulation of the TGF-β-dependent Smad pathway in animal models has provided strong evidence for a role for TGF-β in fibrotic EMT in vivo. EMT of lens epithelial cells in vivo following injury is completely prevented in Smad3 null mice, while primary cultures of Smad3−/− lens epithelial cells treated with TGF-β are protected from EMT (15). Similarly, in the kidney, Smad3 null mice are protected from experimentally induced tubulointerstitial fibrosis and show reduced EMT and collagen accumulation, whereas cultures of renal tubular epithelial cells from Smad3−/− animals show a block in EMT and a reduction in autoinduction of TGF-β1 (16). In human proximal tubular epithelial cells, increased CTGF and decreased E-cadherin were Smad3-dependent, increased MMP-2 was Smad2-dependent, and increases in α-SMA were dependent on both (17). A recent transcriptomic analysis of TGF-β-induced EMT in normal mouse and human epithelial cells demonstrated, using a dominant negative approach, that Smad signaling was critical for regulation of all tested target genes (18). Non-Smad-dependent pathways implicated in TGF-β-dependent EMT include RhoA, Ras, p38 MAPK, PI3 kinase, Notch, and Wnt signaling pathways. In most cases, stimulation of these co-operative pathways provides the context for induction and specification of EMT within a particular tissue, with Smads representing the dominant pathway, which in some instances may be necessary but not sufficient for induction of full EMT (19).

Reversal of TGF-β1-Induced EMT & Fibrosis

A number of interventions have been demonstrated to lead to the reversal of EMT. BMP-7 reversed TGF-β1-induced EMT in adult tubular epithelial cells by directly counteracting TGF-β-induced Smad3-dependent EMT, and evidence for reversal of renal fibrosis occurring via EMT has been shown in vivo (20). BMP-7 was able to delay EMT in lens epithelium in association with downregulation of Smad2, whereas overexpression of inhibitory Smad7 prevented EMT and decreased nuclear translocation of Smads2 and -3 (21). EMT is ameliorated in Smad3 knockout mice (15, 16), and Smad7, an antagonist of TGF-β signaling, or bone morphogenetic protein-7 (BMP-7) acting in a Smad-dependent manner, can reverse or delay fibrosis in renal and lens epithelia (21, 22). Furthermore, HGF blocks EMT in human kidney epithelial cells by upregulation of the Smad transcriptional co-repressor SnoN, which leads to formation of a transcriptionally inactive SnoN/Smad complex, thereby blocking the effects of TGF-β1 (23). These studies suggest the feasibility of modulating Smad activity as a strategy for counteracting actions of TGF-β to induce EMT. Knowledge of the precise molecular mechanisms mediating TGF-β-induced EMT and its interactions with other signaling pathways will be important for developing strategies to inhibit/reverse EMT without disrupting the beneficial effects of TGF-β signaling.

Investigation of EMT and its Regulation by the BMP Agonist Peptides of the Invention

Investigation of EMT requires the use of panels of markers that describe an EMT profile. Loss of the epithelial phenotype can be clearly defined by loss of expression of specific epithelial proteins, including junction associated proteins (e.g., E-cadherin), cytokeratins, and apical actin-binding transmembrane protein-1 (MUC-1). In particular, loss of E-cadherin is a universal feature of EMT, regardless of initiating stimulus (24), and in some instances, reversal of the invasive mesenchymal phenotype can be observed if E-cadherin is produced (25). It has been shown that hyperglycemic conditions can induce EMT in human renal epithelial cells, which become more elongated, adhere less to the substrate and lose their apical-to-basal polarity. During this process, the cells show increased de novo expression of TGF beta, loss of E-cadherin expression (26) and synthesis of extracellular matrix molecules, such as fibronectin and collagen, which are features consistent with a more fibroblast-like phentoype. The activation of myofibroblasts plays a critical role in the processes of cell adhesion, actin re-organization and enhanced cellular progression of chronic renal fibrosis. Idiopathic pulmonary fibrosis (IPF) is a chronic dysregulated response to alveolar epithelial injury with differentiation of epithelial cells and fibroblasts into matrix-secreting myofibroblasts through the EMT process and resulting in lung scaring. During this process, cells show increased de novo expression of TGF beta and lost E-cadherin expression causing myofibroblast activation and collagen production, thereby resulting in pulmonary fibrosis (27, 28, 29).

BMP-7 has been shown to interfere with the TGF-beta signaling pathways thereby leading to reversal of the EMT process, myofibroblast expansion and epithelial cell apoptosis. This effect has considerable benefit in the treatment of renal fibrosis (30) in animal models. The peptides discussed herein specifically interact with both type II and selectively type I BMP receptors and induce BMP signaling, thereby inducing cellular responses that mimic the effects of BMP7, except that of osteogenetic induction. Many, but not all, of these compounds inhibit the EMT process in renal tubular epithelial cells that have been subjected to hyperglycemic conditions. EMT is an essential mechanism in the development of tubulo-interstitial fibrosis. When human proximal tubule epithelial cells are exposed to high glucose, a significant loss of E-cadherin expression is detected Like BMP-7, these compounds effectively prevent the loss of E-cadherin expression in these cells. These results thus explain the importance of BMP signaling properties and the renal protective effects of these peptide agonists.

Control of EMT through the activation of BMP signaling is important for lung regenerative events, but in pulmonary fibrosis it is significantly perturbed (31). Accordingly, the use of these peptide agonists of the BMP pathway to rescue BMP signaling activity represents a strategy of great therapeutic potential for treating human pulmonary fibrosis. These conclusions may be extended to other fibrotic conditions that form fibrosis via EMT.

Potential Use of Peptide Agonists for the Treatment of Fibrotic Diseases

The serine-threonine signaling pathway consists of at least two competing sets of receptors and intra-cellular messenger molecules. On the TGF-beta side the TGF-beta type II receptor, type I receptors ALK 2 and 3 and SMADs 1 and 5 act to promote the EMT process and fibrogenesis, while on the BMP side the BMP type II receptor, type I receptors ALK2, 3 and 6, and SMADs 1, 5 and 8 act to promote the differentiated, epithelial state. Furthermore, these two states tend to stabilize themselves by down regulating signaling entities of the opposing state. Stimulation of cells with TGF-beta side has the effect of down regulating expression of BMPs, BMP receptors and SMADs 1, 5 & 8 and vice versa. Thus, these two pathways act as a bistable biochemical switch. Strategies for treating and/or reversing fibrosis can either be directed toward inhibiting the TGF-beta side (TGF-beta antagonists and inhibitors such as anti-TGF-beta anti-bodies, decoy receptors and TGF-beta binding proteins), or can be directed toward stimulating the BMP side using BMP-7 or other agonists of the BMP pathway such as these peptides.

Treatment of renal tubule epithelial cells with TGF-beta1 induces EMT (E-cadherin expression is decreased while expression of mesenchymal markers such as alpha-SMA, fibronectin, collagen I and CTGF are increased. BMP-7 inhibits all these effects in a dose-dependent manner. In fact, BMP-7 can reverse TGF-beta1-induced EMT resulting in reexpression of endogenous E-cadherin (32). In several animal models of chronic kidney injury, BMP-7 attenuates progressive loss of kidney function and renal fibrosis (33-36).

Similar observations are made in the cases of fibrotic indications. In an animal model of idiopathic pulmonary fibrosis, BMP-7 has been shown to attenuate experimentally-induced fibrosis in the lungs (31) by modulating TGF beta-induced EMT and inhibiting collagen production by lung fibroblasts (37). So also in the case of liver fibrogenesis where there is evidence that BMP-7 plays an important role as an anti-inflammatory and anti-fibrogenic agent (38). Cardiac fibrosis is associated with the emergence of fibroblasts originating from endothelial cells, suggesting an endothelial-mesenchymal transition (EndMT) similar to events that occur during formation of the atrioventricular cushion in the embryonic heart. Cardiac endothelial cells treated with TGF-beta1 have been observed to undergo an EndMT process, whereas BMP-7 preserves the endothelial phenotype. In mouse models of pressure overload and chronic allograft rejection, systemic administration of recombinant human BMP-7 significantly inhibits EndMT and the progression of cardiac fibrosis (39).

In the case of vascular calcification associated with chronic renal fibrosis, the results of several studies imply that BMP-7 would also be an effective treatment. Cells exhibiting an osteoblast-like phenotype in the vessel wall may be important in the etiology of vascular calcification. Expression of osteocalcin is used as a marker of osteoblastic function. It has been shown that osteocalcin increases in untreated uremic animals, but is down-regulated to levels similar to non-uremic control animals when they are treated with BMP-7 (40). In all above fibrotic diseases, EMT has been recognized as an integral aspect of tissue fibrogenesis, and stimulation of BMP signaling has been shown to be effective at inhibiting or even reversing the EMT process.

Given that the peptides in this application are shown to be effective agonists of BMP signaling pathway inhibiting the effects on human renal proximal tubule epithelial cells that of high D-glucose (hyperglycemic condition) induced EMT, in association with loss of E-cadherin expression. Furthermore, these peptides have been shown to reverse the EMT induced by TGF-beta1, resulting in re-expression of endogenous E-cadherin and preservation of epithelial morphology (see Figures—from Nature Medicine article, 41). In several animal models of chronic kidney injury, one of these peptides, administered orally, attenuated progressive loss of kidney function and renal interstitial fibrosis. In an animal model of idiopathic pulmonary fibrosis, the peptide agonist effectively inhibited bleomycin-induced EMT of lung epithelial cells and pulmonary fibrosis (See Example 7 and related Figures). THR-123 treated mice (daily oral dose of 5 mg/kg) had an 80% survival rate out to sacrifice at 16 days, whereas vehicle treated mice had a 100% mortality by 8 days. Histomorphometric analysis of lung tissue demonstrated significantly less pulmonary fibrosis in the THR-123 treated animals Likewise, these peptide agonists of BMP signaling pathway offer therapeutic potential for the treatment of other fibrotic diseases such as liver cirrhosis, atherosclerosis, cardiac fibrosis and scleroderma-renal risk (systemic sclerosis).

Likewise, peptide agonists of BMP signaling which are antagonists of TGF-β actions and tissue fibrosis may offer potential therapeutic use for the treatment of other fibrotic diseases such as liver cirrhosis, atherosclerosis, cardiac fibrosis and scleroderma-renal risk (systemic sclerosis). Several cellular assays for screening and animal models are under consideration to test the compound(s) efficacy in these fibrotic diseases. See Tables described below for Template.

Template for Screening BMP-Agonist and Antagonist Peptides of the Invention:

Template A: Cellular models (In vitro assays) of fibrotic
diseases for testing peptides of the invention:.

| Fibrotic disease | Cellular model | End point(s) |
|---|---|---|
| Chronic Kidney Disease | EMT assay. D-glucose | E-cadherin, Vimentin, |
| (CKD): Renal fibrosis | induced epithelial phenotype | pSmad2 and pSmad1/5/8 |
| | loss in human proximal tubule | expression by |
| | epithelial cells (HK-2), | immunofluorescence and |
| | prevention of EMT by | Western-blot methods. |
| | compound | |
| Idiopathic Pulmonary | EMT assay: TGF beta induced | E-cadherin, Vimentin, MMP- |
| Fibrosis | EMT in human bronchial | 2, pSmad2 and pSmad1/5/8 |
| | epithelial cells (HBECs) from | expression by |
| | Lonza co. Assay is based on | immunofluorescence and |
| | mechanism of bronchial | Western-blot methods. |
| | epithelial cell to mesenchymal | |
| | cell differentiation, which, in | |
| | turn, can contribute to lung | |
| | fibrosis | |
| Liver Cirrhosis | EMT assay: TGF beta induced | TGF-β1 induced FSP1 |
| | EMT in primary mouse | expression. In combination |
| | hepatocytes. Assay is based on | with TGF-β1, |
| | TGF beta -induced EMT in | BMP7/compound prevents |
| | hepatocytes causing change to | loss of albumin expression and |
| | mesenchymal phenotype | with no detectable FSP1 |
| | which, in turn, can contribute | expression. pSmad2 and |
| | to liver fibrosis | pSamd 1/5/8 expression |
| | | by immunofluorescence and |
| | | Western-blot methods |
| Cardiac fibrosis | TGF beta induced Endothelial- | Alpha-smooth muscle actin |
| | to-mesenchymal transition | (α-SMA) staining. Increased |
| | (EndMT) of mouse cardiac | protein levels of α-SMA, Snail |
| | endothelial cells (MCECs) | and β-catenin upon TGF beat |
| | contributing to the | induced EndMT & pSmad2 |
| | pathogenesis of cardiac | and pSamd 1/5/8 expression |
| | fibrosis | by immunofluorescence and |
| | | Western-blot methods |
| Atherosclerosis | Cell based assay using primary | Expression of SMC-specific |
| | human aortic smooth muscle | markers., namely alpha-actin |
| | (HASM) cells. The assay is | and heavy chain myosin |
| | based on BMP-7 stimulation | |
| | of expression of SMC- | |
| | specific markers., namely | |
| | alpha-actin and heavy chain | |
| | myosin | |
| Scleroderma-renal | No established cell based | — |
| risk | assay | |

Template B: Animal models (In vivo assays) of fibrotic diseases
for testing peptides ot the invention for anti-fibrotic activity.

| Fibrotic disease | Animal model | End point(s) |
|---|---|---|
| Chronic Kidney Disease | Unilateral Ureteral | sCr, BUN, Hematoxilin-Eosin |
| (CKD): Renal fibrosis | Obstruction (UUO) model | and Masson's trichrome |
| | | staining of lung sections; P- |
| | | smad-2, P-smad1/5/8, EMT |
| | | (E-chaderin, Vimentin), |
| | | collagen, macrophages |
| | | infiltration by ED-1 |
| Idiopathic Pulmonary | Experimental model of | Hematoxilin-Eosin and |
| Fibrosis | pulmonary fibrosis induced by | Masson's trichrome staining |
| | a single intra-tracheal dose of | of lung sections; P-smad-2, P- |
| | bleomycin dose of bleomycin | smad1/5/8, EMT (E-chaderin, |
| | in 6- to 8-wk-old mice. | Vimentin), collagen type Ia1, |
| | | MMP-2 and TGF beta |
| | | expression |
| Liver Cirrhosis | Experimental model of liver | Hematoxylin and eosin, and |
| | fibrosis induced by | Masson's trichrome staining |
| | intraperitoneal dose of CCl4 | of liver sections. psmad2, |
| | in C57BL/6 mice. | psmad1/5/8, EMT (Vimentin), |
| | | Collagen III and α -smooth |
| | | muscle by immune-fluorescent |
| | | staining. |
| Cardiac fibrosis | Mouse models of pressure | Hematoxylin and eosin, and |
| | overload and chronic allograft | Masson's trichrome staining |
| | rejection for cardiac fibrosis | of liver sections. psmad2, |
| | | psmad1/5/8, EndMT (α-SMA |
| | | and Snail), Collagen and α - |
| | | smooth muscle by immuno- |
| | | fluorescent staining. |
| Atherosclerosis | Uremia is imposed on LDL | Hematoxylin and eosin |
| | receptor null mice for a model | staining. Imunohistochemistry |
| | of atherosclerosis, The model | of proximal aortic sections for |
| | involves a two-step procedure | osteocalcin. α-SMA staining, |
| | to create uremia. Briefly, | total aortic calcium content, |
| | electrocautery is applied to the | staining of aortic outflow tract |
| | right kidney through a 2-cm | sections for calcification |
| | flank incision at 10 wk old and | (Alizarin Red-S). Serum for |
| | performed left total | BUN |
| | nephrectomy 2 wk later. | |
| Scleroderma-renal | A mouse model of GVH- | Dermal thickening, |
| risk | induced Systemic Sclerosis | Morphometric analyses of |
| | | connective tissue and vascular |
| | | changes by. standard |
| | | Trichrome histochemical |
| | | Staining. Collagen deposition, |
| | | vasoconstriction, and |
| | | parameters of immunity, |
| | | inflammation in skin and |
| | | internal organs, and |
| | | autoantibody generation. |
| | | Immunohistochemical staining |
| | | for type III collagen, anti- |
| | | αSMA, and endothelin 1 (ET- |
| | | 1), type VII collagen, |
| | | macrophages, CD4_ T cells, |
| | | and CD8_ T cells, |

### Example 6

### In Vitro and In Vivo Assays to Test for Anti-Fibrotic Activity of the Peptides of the Invention in Pulmonary Fibrosis Model

A. In Vitro Assay (Prophetic)

Objective:

Compounds were screened in vitro for anti-fibrotic activity (inhibition or reversal of the EMT process) using human bronchial epithelial Cells (HBECs). This assay measures the ability of test compounds to inhibit the TGF-β-mediated down-regulation of E-cadherin expression (an epithelial marker), and the up-regulation of several mesenchymal markers, i.e., Vimentin and alpha-smooth muscle actin (alpha-SMA). Furthermore, the same cells are used to examine whether EMT inhibition/reversal by test compounds is Smad-dependent, a primary process in the BMP signaling mechanism.

The assay is also used to optimize the active compound. The expression (up or down) of epithelial marker E-cadherin, and mesenchymal markers N-cadherin, vimentin, alpha-smooth muscle actin (alpha-SMA), MMP-2, MMP-9 and Collagen Type I alpha 1 (COL1A1) in response to compounds are measured by Western-blot analysis, and then by quantitative ELISA assays.

Experimental Design and Methods:

Cell Culture:

Human bronchial epithelial cells (HBECs; Lonza, MD, USA) are maintained in BEGM medium (Lonza), at 37° C. in the presence of 5% CO₂ in a humidified incubator. All further experiments with HBECs are performed in BEGM medium only, unless indicated otherwise.

EMT Assay:

HBECs are seeded at a density of 10⁶ cells/well in 1:100 BEGM:BEBM (six-well plates). Cells are allowed to adhere for 1 day and then changed into media containing 5 ng/ml of TGF-β1 (R&D Systems, MN, USA). The HBECs are then allowed to differentiate for 3-5 days. Human BMP 7 recombinant protein can be used as a positive control in the assay. For EMT assay in the presence of test compounds, HBECs are incubated with increasing concentrations (1 to 200 μM) of test compound for 1 h prior to EMT induction, which is initiated by the addition of TGF-β1 (5 ng/ml) and incubation for 48 h or 3-5 days The Smad pathway inhibitor SB431542 (10 μM, Sigma-Aldrich) and the ERK pathway inhibitor PD98059 (10 μM, Calbiochem) can also used as reference inhibitors.

Phase Contrast Microscopy:

HBECs incubated in the presence of TGF-β1 can be evaluated by phase contrast microscopy. Treatment with TGF-β generally results in loose cell-cell contact, and cells that become more sparse and change into an elongated, fibroblastic morphology. Anti-fibrotic compounds are expected to prevent these morphological changes.

Immunofluorescent Staining and Microscopy:

As described above, HBEC cells are plated in wells and are exposed to the test compound for 1 hr prior to the addition of TGF-β1 (5 ng/ml) (EMT induction) for 48 h or 3-5 days. The cells are washed, fixed in 1:1 acetone: methanol mixture for 2 minutes at room temperature (RT), and blocked in PBS containing 1% goat serum and 1% BSA for 7 minutes at RT. To identify the presence of phosphorylated SMAD1/5/8 and or phosphorylated SMAD 2/3, and their translocation into cell nuclei, the cells are then immunostained, first with a primary rabbit antibody specific to phosphorylated SMAD1/5/8 or phosphorylated SMAD 2/3 and second with a FITC-tagged secondary antibody against rabbit IgG. The immunostained cells are visualized and photographed using an inverted fluorescent microscope (Axiovert; Carl Zeiss), at 200× magnification (20× objective with 10× ocular).

Western Blotting:

To determine the up or down-regulation of epithelial and mesenchymal markers the following antibodies were used in a Western Blot format: E-cadherin (Abcam, MA, USA), N-cadherin (Zymed, CA, USA), vimentin (Abcam), α-SMA (Sigma), MMP-2 (R&D Systems), pSmad2 (Cell Signaling, MA, USA), and pSmad1/5/8 (Cell Signaling). Incubated cells are lysed in TRIS buffer containing 1% NP-40, 150 mM NaCl, 50 mM Tris pH 8.0, 1 mM sodium orthovanadate, 5 mM NaF and a protease inhibitor cocktail (Sigma, NY, USA). Cell lysates are then subjected to SDS PAGE on a 4-10% gradient acrylamide gel. The electrophoresed protein bands are then transferred onto PVDF membranes and blocked in Tris-buffered saline (TBS), 0.1% Tween-20, 5% non-fat dried milk for 1 h at room temperature (RT). The blots are then washed three times in TBS 0.1% Tween (TBS-T) and incubated for 1 h at RT with a horseradish peroxidase-labelled secondary antibody (Invitrogen). After repeated washing in TBS-T the immunoreactive proteins are detected by chemiluminescence (ECL; Pierce, L, USA) according to the manufacturer's instructions. An antibody against GAPDH is used as loading control.

ELISA Assays:

Dose-related anti-fibrotic response of HBEC cells to test compound can be determined by ELISA assay using antibody against the epithelial marker E-cadherin (#7886, Cell Signaling Technology, Inc., MA, USA), and to detect mesenchymal markers, antibodies against MMP-2 (DMP200, R&D Systems, MN, USA), MMP-9 (DY911, R&D Systems, MN, USA), Alpha SMA (ACTA2, antibodies-online Inc, Atlanta, Ga. 30346, USA), N-cadherin (ABIN867238, antibodies-online Inc, Atlanta, Ga., USA), human vimentin (ABIN869687, antibodies-online Inc, Atlanta, Ga., USA), or human Collagen, Type I, alpha 1 (COL1A1) (ABIN512856, antibodies-online Inc, Atlanta, Ga., USA).

Validation of the Assay:

The data demonstrate that primary human bronchial epithelial cells (HBECs) are able to undergo EMT in response to transforming growth factor-beta 1 (TGF-β1), as revealed by typical morphological alterations and EMT marker progression at the protein level by western blot and quantitative ELISA methods. An increased expression of several mesenchymal markers including N-cadherin, vimentin, MMP-2 and of the myofibroblast marker a-SMA was detected at the protein level. In contrast, downregulation of the epithelial marker E-cadherin, as well as an enhanced expression of the metalloprotease MMP-2 was observed in the presence of TGF-β1. This effect was also shown as primarily mediated via a Smad 2/3 dependent mechanism and can be further modulated by BMP pathway activation.

Demonstration of Compound Activity:

In order to determine whether a compound(s) of the invention may also play a role in fibrosis pathogenesis of the airways via modulating EMT in HBECs, HBECs were incubated with the test compound alone or before inducing EMT with TGF-β1. The test compound(s) were found to inhibit collagen I expression induced by TGF-β1. Moreover, overexpression of the metalloproteases MMP-2 and MMP-9 induced by TGF-β1 was inhibited by the test compound(s). An antagonistic effect of the test compound upon the TGF-β1-mediated upregulation of MMP2 protein was further confirmed by assessing cellular morphology using phase contrast microscopy. The test compound was effective to inhibit TGF β induced increased expression of several mesenchymal markers including N-cadherin, vimentin, MMP-2 and of the myofibroblast marker a-SMA. This is associated with the marked regain of epithelial phenotype E-cadherin. In order to assure that BMP signaling is active in the presence of the test compound, an antibody is used that cross-reacts with the phosphorylated forms of Smads 1, 5 and 8, downstream effectors of BMP signaling. Increased phosphorylation of Smad1/5/8 was found in the presence of the test compound, an effect that could be abrogated if TGF-β1 is concomitantly added. These results suggest a counteraction by the test compound(s) over TGF-β and its pathway during EMT in HBECs.

The results thus provide the basis of further investigations into the mechanism of bronchial epithelial cell to mesenchymal cell differentiation during lung fibrosis, and for the development of new therapeutic approaches for pulmonary fibrosis.

B. In Vivo Assay (Reduced to Practice)

Objective:

Test compounds found to be active in the in vitro pulmonary fibrosis assay described above are then tested in an animal model where pulmonary fibrosis in mice is induced by a single intra-tracheal dose of bleomycin. The endpoints in this model are survival and histomorphometric evaluation of the extent of fibrosis in lung tissue. The study is carried out in compliance with the guidelines of the Animal Welfare Act Regulation, 9 CFR 1-4.

Materials & Methods:

Formulation of the Test Compound:

The test compound, in lyophilized form, is dissolved in 50 mM acetate buffer, pH 4.5 at an initial concentration of 20 mg/mL. The stock solution is then divided into several one mL aliquots, snap frozen and stored at −70° C. until used. On the Day of use, each aliquot is thawed and further diluted to a required working concentration in PBS, pH 7.5. The working concentration is determined from the intended dose (mg/kg body weight) and the oral administration volume.

Materials:

1. BALB/c mice mice (Charles River Laboratories, Cambridge) at a weight of 21-26 g

2. Bleomycin (Blenoxane, Sigma, St. Louis, Mo.)

3. Cyclophosphamide (as the mono-hydrate from Sigma-Aldrich, St. Louis, Mo.).

Experimental Design:

Animal Model:

Studies to evaluate the degree to which test compounds (Seq ID No. 1-11) are capable of ameliorating bleomycin-induced lung fibrosis are performed on BALB/c mice (Charles River), which are maintained on a normal diet under standard animal house conditions. To initiate pulmonary fibrosis, mice are anesthetized by an intraperitoneal injection of 250 μl of 12.5 mg/ml ketamine followed by intratracheal instillation of 2 U/kg body weight of Bleomycin (Blenoxane, Sigma, St. Louis, Mo.) in 50 μl sterile PBS. In addition, the mice are administered a single intraperitoneal injection of cyclophosphamide (150 mg/kg of body weight). The animals challenged with Bleomycin plus Cyclophosphamide are separated into two groups, each group having a minimum of 6 animals. The vehicle treated group (Group 1) receives daily oral doses of PBS, pH 7.5. The compound treated group (Group 2) receives the compound being tested by daily oral doses of 5 mg/kg body weight in the preliminary study, and in a follow up study at dose levels of typically 0.03, 0.1, 0.3 and 1.0 mg/kg to establish the dose response and to determine the minimum effective dose. These treatments are continued for up to 16 days after Bleomycin administration. A third control group of mice receive intra-tracheal PBS rather than Bleomycine and Cyclophosphamide.

Preparation of Lungs and Histological Assessment of Pulmonary Fibrosis:

After completion of the in-life study, the animals are euthanized (methoxyflurane anesthesia), and the lungs are perfused with ice cold Hank's balanced salt solution to remove blood-born cells, and then inflated under a constant pressure of 30 cm H₂O with 10% normal buffered formalin (NBF). Lungs are ligated at the trachea, removed en bloc, and immersed in NBF for 24 hr. After which, tissue samples are changed to 70% alcohol before paraffin embedding, followed by sectioning and staining with hematoxylin, eosin and Masson's Trichrome. Sections are analyzed microscopically to evaluate pulmonary fibrosis by determining the degree of collagen accumulation (Masson's Trichrome).

Smad Signaling:

To measure the level of phospho-Smad 1, 5, 8 (BMP signaling) or phospho Smad 2/3 (TGF-beta signaling), slides from each group of mice are stained separately. Sections are deparaffinized, rehydrated, and subject to antigen retrieval. Subsequently, endogenous peroxidase was quenched with 3% H₂O₂ and blocked for 20 min with 50% goat serum. Primary phospho-Smad 1,5,8 or phospho-Smad 2,3 antibody (rabbit polyclonal, Cell Signaling Technology, Danvers, Mass.) is added to the respective section group and incubated overnight at 4° C. in 25% goat serum. Sections are then incubated with a biotinylated goat anti-rabbit secondary antibody (Vector Labs Burlingame, Calif.) for 60 min followed by a 10 min treatment with Streptavidin-HRP (Dako, Mississauga, ON). The antigen of interest is visualized by using the brown chromogen 3,3-diaminobenzidine (Dako, Mississauga, ON) and counterstained with Harris Hematoxylin Solution (Sigma, Oakville, ON).

Lung Immunohistochemistry for EMT:

To evaluate the presence of epithelial and/or mesenchymal markers, lung tissue sections are dewaxed, rehydrated, blocked with 10% goat serum for 60 min at room temperature and immunofluorescently stained for α-SMA or Vimentin (mesenchymal markers) or E-cadherin (epithelial marker). Sections were incubated with anti-α-SMA, or anti-vimentin or co-incubated with anti-E-cadherin antibody overnight at 4° C. and subsequently incubated with goat anti-mouse IgG-TRITC antibody or goat anti-rabbit IgG-FITC antibody, as appropriate, for 1 hour. To identify nuclei DAPI is used to stain nuclei (500 ng/ml in 95% ethanol) for 20 sec, and coverslips are mounted with 80% glycerol. Slides are examined using a fluorescence microscope equipped with a digital camera.

TUNEL Assay:

Apoptotic cells are detected by using a TUNEL detection kit (In Situ Cell Death Detection Kit, Roche Applied Science). Tissue sections are deparaffinized, rehydrated, and washed with distilled-deionized water. After treatment with proteinase K, fragmented DNA is labeled with fluorescein-dUTP, using terminal transferase. Slides are mounted with DAPI containing Vectashield. Sections are analyzed using a fluorescence microscope equipped with a fluorescence detection system. The apoptotic percentage is obtained by manual counting of TUNEL⁺ cells in groups of 4,000 or more cells.

Tissue Homogenate Preparation:

Collagen Assay:

Lungs from all groups of mice are homogenized in complete protease inhibitor (Roche Diagnostics Corp, Indianapolis, Ind., USA). Homogenates are centrifuged at 900×g for 10 minutes and frozen until time of analysis.

Sirius red reagent is added to each lung homogenate (50 ml) and mixed for 30 minutes at room temperature. The collagen-dye complex is precipitated by centrifugation at 16,000 g for 5 minutes and the pellet resuspended in 1 ml of 0.5 M NaOH. The concentration of collagen in each sample is measured as absorbance at 540 nm and values interpolated from a known standard curve as per manufacturer's instructions.

Chemokine, MMP-2 and MMP-9 levels were measured using ELISA kits from R& D Systems, CA, USA.

Results:

Animal Mortality:

For vehicle treated mice (Group 1), mortality is 100% by 8 days after administration of Bleomycine and Cyclophosphamide. For the compound treated mice that were challenged with Bleomycine+Cyclophosphamide (Group 2), survival is as high as 80% at 16 days after initiation of fibrosis (end of in-life study) whereas vehicle treated mice had a 100% mortality by 8 days.

Lung Fibrosis:

Pulmonary fibrosis was evaluated histomorphometricly by determining the percentage of field with collagen accumulation lung sections stained with Masson's Trichrome. Ten days after treatment with Bleomycine and Cyclophosphamide, vehicle treated animals showed increased lung fibrosis with a score of 31%. This was associated with the death of all these animals. However, THR-123 when given orally reduced Bleomycine+Cyclophosphamide induced lung fibrosis to 16% by the day 16, with the survival of all mice.

Other Expected Results:

Lungs in bleomycine-treated mice show EMT process, as revealed by EMT marker progression in lung tissue sections. An increased expression of several mesenchymal markers including N-cadherin, vimentin, and of the myofibroblast marker a-SMA was observed. In contrast, downregulation of the epithelial marker E-cadherin, as well as an enhanced expression of the metalloprotease MMP-2 are observed in these sections. We have also observed that this effect is primarily mediated via a Smad 2/3 dependent mechanism and can be further modulated by BMP pathway activation.

Treatment with the compounds resulted in a significant increase in survival and inhibition of bleomycine-induced increases in expression of several mesenchymal markers including N-cadherin, vimentin, MMP-2 and the myofibroblast marker a-SMA. This is associated with a marked regain of epithelial phenotype E-cadherin expression, indicating the prevention of EMT. Also, bleomycine treated animals that were treated with compound show increased phosphorylation and their nuclear translocation of Smad 1/5/8 in lung tissues, indicating involvement of the primary BMP signaling pathway. Moreover, the increased expression of collagen, and metalloproteases, MMP-2 and MMP-9 that are observed in mice treated with bleomycine, are all inhibited in animals with the compounds.

These results (including expected), in this widely used animal model of pulmonary fibrosis, are evidence that the compounds tested can alleviate pulmonary pathology by markedly reducing fibrosis, and consequently significantly improving animal survival.

### Example 7

### A BMP Agonist Peptide of the Invention Reverses Fibrosis and EMT in Kidney Tubular Epithelium Cells Via BMP7 Signaling Pathway and the Alk-3 Receptor

Molecules associated with TGFβ superfamily such as BMPs and TGFβ are key regulators of inflammation, apoptosis and cellular transitions. Here, it is demonstrate that a BMP7 receptor, activin-like kinase 3 (Alk-3), is significantly elevated in response to kidney injury and its specific deletion in the kidney tubular epithelium leads to accelerated TGFβ/Smad3 signaling, tubular epithelial injury and kidney fibrosis, suggestive of a reno-protective role for Alk-3 mediated signaling. To exploit this activity therapeutically, a structure-function analysis of ALK-3/BMP ligand-receptor complex is performed by employing synthetic organic chemistry to construct orally available, small cyclic BMP peptide mimics with specific binding to Alk-3 receptor. Screening identified a peptide, THR-123 (SEQ ID NO: 1 of Table 1), that inhibits inflammation, apoptosis and epithelial to mesenchymal transition program in several different in vitro and in vivo experiments. THR-123 suppressed and reversed renal injury and fibrosis in five different mouse models, and a combination of THR-123 with angiotensin-converting enzyme inhibitor, captopril, exhibited additive therapeutic benefit in controlling kidney fibrosis. Our results demonstrate that THR-123 is a novel anti-fibrosis agent with a potential utility in the clinic to reverse fibrosis.

### Introduction:

Bone morphogenetic protein-7 (BMP7), a member of the transforming-growth factor (TGF)-β superfamily, acts as an antagonist of TGF-β-mediated fibrogenic activity¹⁻³. BMP7 binds to activin-like kinase (Alk)-2, -3, -6 and displays distinct activities in different cell types⁴, exhibiting anti-inflammatory and anti-apoptotic functions as well as promoting bone formation^{5,6}. From a functional point of view, anti-fibrotic activity of BMP7 is an attractive candidate for testing in the clinic but its multiple activities (especially bone formation) via different receptors create certain clinical development challenges. In this regard, various studies have suggested that the bone forming action of BMP7 is mediated exclusively via Alk-6 and the anti-fibrotic action is likely mediated via Alk-3 and possibly via Alk-2⁷⁻¹². BMPs signal via Smads 1/5, while TGFβ signals via Smad 2/3⁴.

End stage renal disease (ESRD) due to many different etiologies exhibit a positive correlation with the degree of tubulo-interstitial fibrosis¹³⁻¹⁷. Here it is demonstrated that Alk-3 functions to inhibit fibrosis by controlling inflammation, apoptosis and EMT program. A small cyclic peptide (THR-123) that mimics BMP7 activity, binds to Alk-3 and reverses kidney fibrosis. THR-123 is a novel therapeutic agent against progressive fibrotic kidney diseases, for which specific and effective therapy is not available.

### Results:

(A) Alk-3 Receptor on Tubular Epithelial Cells Serves as a Negative Regulator of Fibrosis

Reno-protective properties and anti-fibrotic effects have been demonstrated for BMP7 in various kidney disease models^{2,18-20}. Some studies have suggested that BMP7 expression is suppressed in acute and chronic kidney injury^{21-24.} We screened for expression levels of the several molecules regulated by BMP7 in mice with chronic renal injury at different time points. Among the 13 different BMP7 regulated molecules evaluated, only Alk-3 expression peaked after 1 week of kidney injury (FIG. 42 A). Between 3-6 weeks, Alk-3 expression remained high when compared to control kidneys (FIG. 42 A). At nine weeks, Alk-3 expression decreased when compared to control kidney (FIG. 42 A). BMP7 expression levels decrease the most among all the molecules tested following kidney injury, reaching its minimum level at 3 weeks after injury and remained low until week 9 (FIG. 42 A).

BMP7 binds to Alk-3 and phosphorylates receptor regulated Smads 1/5⁴. In mice with NTN induced chronic kidney fibrosis (FIG. 42 B-D), phosphorylated Smad1 (pSmad1) accumulates in the nucleus of the tubules at 1 week following injury is detected. Interestingly, the pSmad1 decreased again around week 6 post kidney injury (FIG. 42 E-G), thus presenting a similar trend as that observed with Alk-3 expression (FIG. 42A). These results further suggested that BMP7/Alk-3 axis correlates negatively with renal epithelial injury and interstitial fibrosis. To functionally address this observation, we deleted Alk-3 receptor in the tubular epithelial cells using γGT-Cre mice²⁵ and mice with floxed allele of Alk-3²⁶.

Six weeks after the induction of NTN in control mice, fibrosis in these mice was less compared to fibrosis in γGT-Cre; Alk-3 fl/fl mice (Alk-3 deleted mice) (FIG. 42 T-V). Such accelerated fibrosis in the Alk-3 deleted mice is associated with enhanced activation of TGF-β pathway, as demonstrated by the increased pSmad2 in the nucleus of the tubular epithelial cells (FIG. 43). Renal function as judged by serum BUN measurement was significantly higher in the Alk3 deleted mice with fibrosis when compared to the control mice (FIG. 42 W). Collectively, these results suggested that Alk-3 may play a critical role in protecting the renal interstitium against fibrosis.

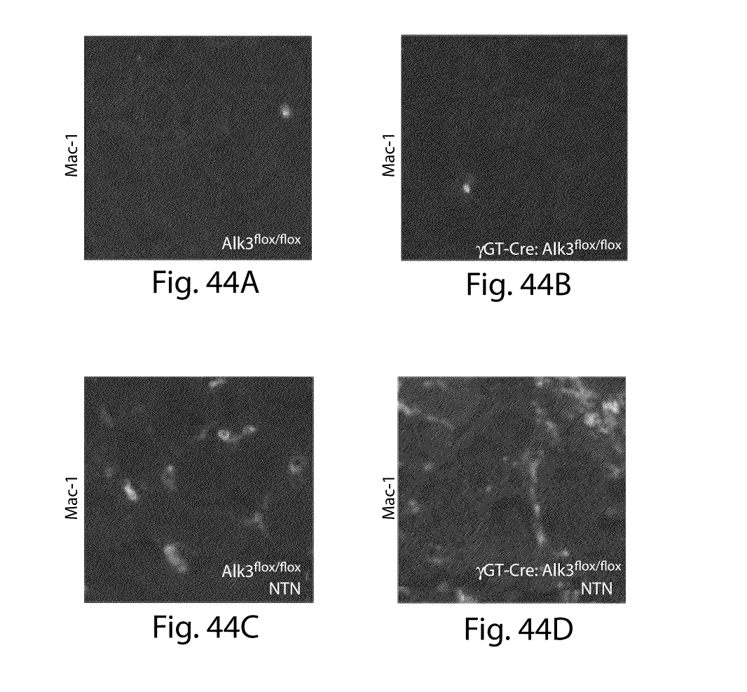

Inflammation associated with macrophage influx and renal epithelial apoptosis are considered to be important instigators of renal fibrosis²⁸. Importantly, previous studies have demonstrated the importance of BMP7/Alk-3/Smad1/5 signaling pathway in controlling inflammation, apoptosis and EMT program in the kidney. We demonstrated that fibrosis in the context of Alk3 deleted mice in the kidney tubular epithelium resulted in increased influx of MAC-1 positive macrophages (FIG. 44), and increased number of tubular epithelial cells exhibited co-localization of both epithelial marker E-cadherin and mesenchymal marker, FSP1/S100A4, indicative of the EMT program within those cells (FIG. 42 X-Z).

(B) Design of the BMP Signaling Pathway Agonist, THR-123

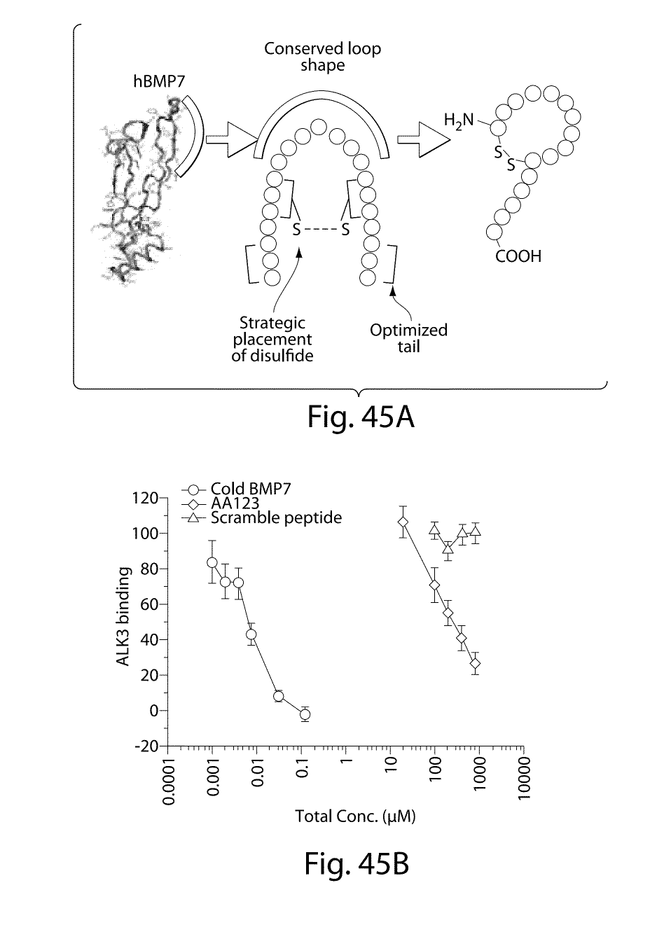

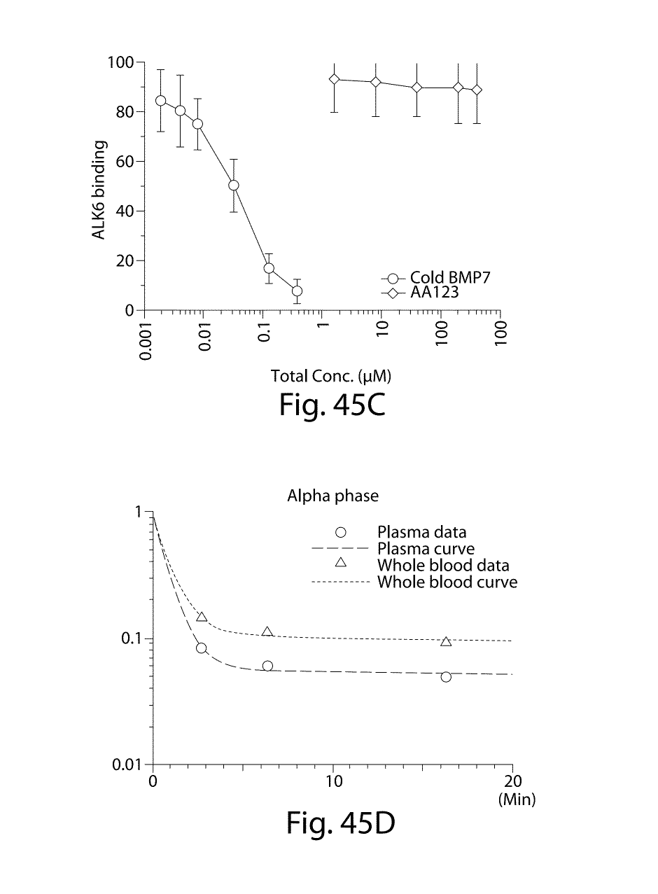

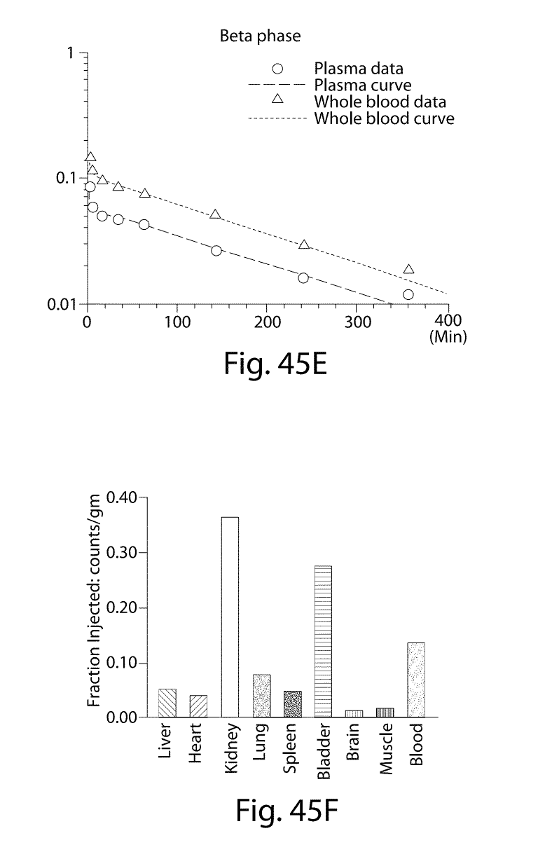

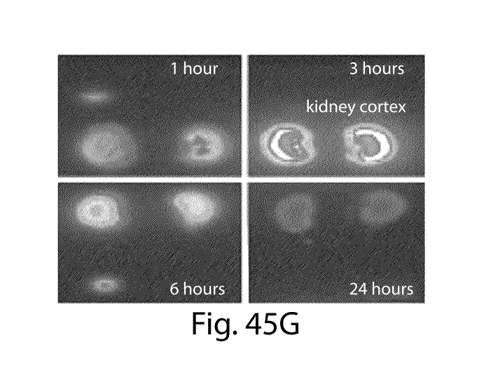

Cyclic peptide agonists of the BMP signaling pathway were designed by identifying regions of the 3D structure of TGF-β2^{29,30} and BMP7³¹ most likely involved in receptor interactions by comparing side chain solvent accessibility with the regions of the TGF-β super family aligned sequences having the highest variability³¹. To further refine the regions of interest, a structure-variance analysis (SVA) program³² was used that weighs physical-chemical residue properties at each position based on their correlation with an activity. The goal was to identify receptor-binding regions and then optimize the sequence for specific BMP activities. The highest scoring residue positions were then mapped onto the 3-D structure of BMP-7³¹. Of the three structural regions identified³¹, peptides designed around the finger 2 loop proved to be the most promising. These are 16 residue long peptides of ˜20 kDa molecular weight that are cyclized via a disulfide bond between the first and 11^{th} residue positions in order to stabilize a loop similar in conformation to the finger 2 loop of BMP7 (FIG. 45 A).

(C) Lead Optimization and Characterization of THR-123

Preliminary screening for optimization was based on anti-inflammatory efficacy in an in vitro cell-based assay using a human renal tubular epithelial cell line (HK-2). The assay tested the ability of compounds to reverse the increase in production of cytokine IL-6 that resulted from stimulation of the cells with tumor necrosis factor (TNF)-α. Sequence-activity analysis was carried out using the SVA program. After six optimization cycles, THR-123 (FIG. 2A) emerged as the lead compound, which was further evaluated in other relevant anti-fibrosis assays (vide infra).

First, specific affinity of the extra cellular domains (ECD) of the several type I receptors of BMPs to THR-123 was analyzed. Binding of cold BMP7 to immobilized receptor ECDs was determined by competition with ¹²⁵I-labeled BMP7 and analyzed by Scatchard analysis to determine effective dissociation constants of BMP7 for each of the receptor ECDs. To obtain an estimate of the effective dissociation constant of THR-123 for a particular receptor ECD, the dissociation constant for cold BMP7 was multiplied by the ratio of the ED50 of THR-123 to the ED50 for cold BMP7. The data demonstrated that THR-123 competes with BMP7 for Alk-3 (FIG. 45 B) and to a small extent with Alk-2 (data not shown), whereas absolutely no competition was observed on Alk-6 (FIG. 45 C), suggesting that of the three known BMP type I receptors Alk-3 is the predominant target for THR-123.

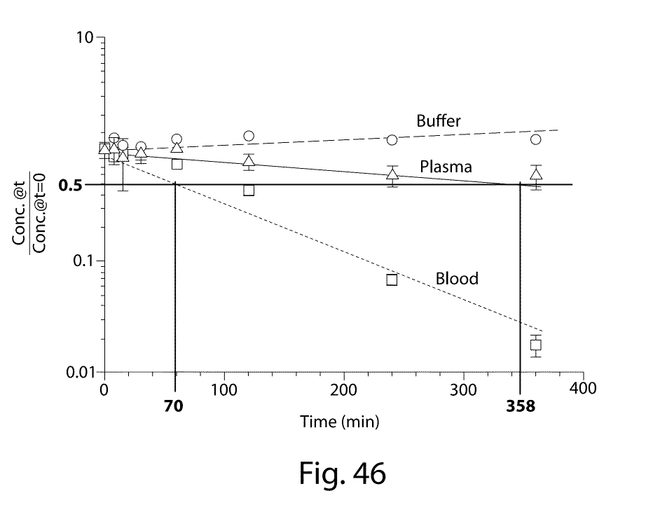

The stability of THR-123 in whole blood and plasma was tested in vitro. In PBS-mannitol buffer, THR-123 was stable for over 400 minutes (FIG. 46). In rat plasma, THR-123 is slowly degraded with a half-life of 358 minutes (FIG. 46), while in whole blood, THR-123 degraded rapidly (half-life of 70 minutes) (FIG. 46).

The persistence of THR-123 in systemic circulation was evaluated using iv-administered ¹²⁵I-labeled compound and following the radioactivity decay. In both plasma and whole blood, THR-123 levels immediately decreased within 5 minutes (by almost 90%), suggesting a very short half-life of THR-123 in the alpha-phase (FIG. 45 D). Beta-phase assessment of ¹²⁵I-THR-123 indicates a half-life of 55-58 min (FIG. 45 E). Six hours after intravenous administration of ¹²⁵I-THR-123, the majority of the radioactivity was still localized in the kidney and bladder (FIG. 45 F), suggesting that THR-123 accumulates in the kidney and is excreted via the bladder into the urine. Orally administrated ¹²⁵I-labeled THR-123 localized primarily to the kidney cortex within one hour after ingestion and peaked at about 3 hours (FIG. 45 G). Twenty-four hours after ingestion, most of the ¹²⁵I-THR-123 radioactivity was completely cleared from the kidney (FIG. 45 G).

(D) THR-123 Inhibited Inflammatory Cytokines Production, Apoptosis and EMT Program of Tubular Epithelial Cells

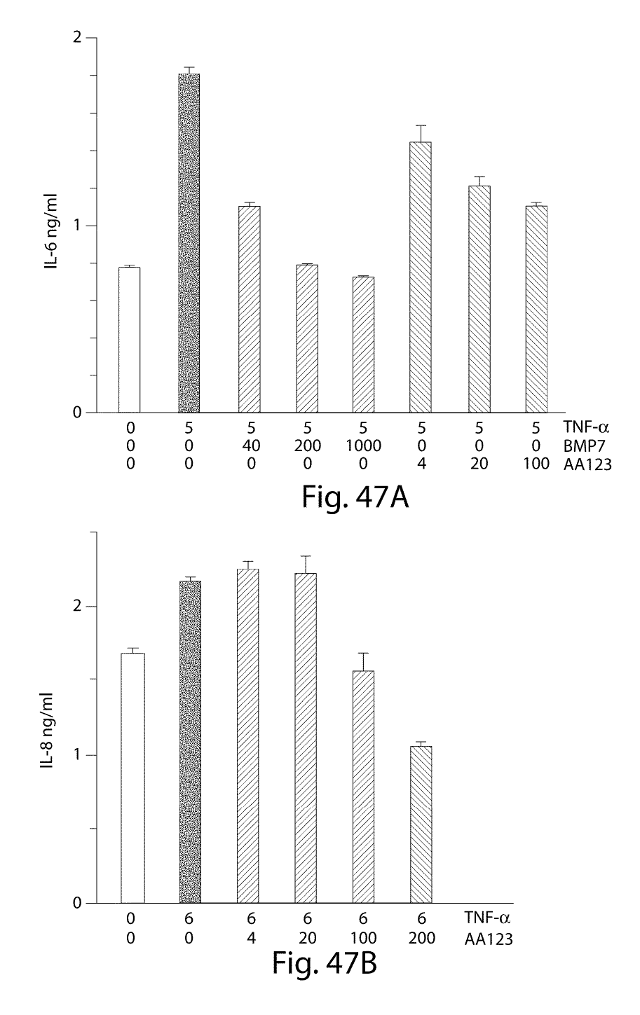

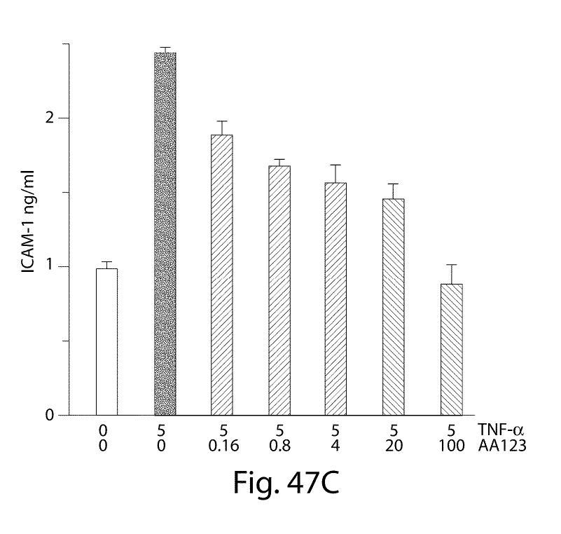

Inflammation is a key feature in renal fibrosis. BMP7 displays an anti-inflammatory activity^{5,33}, thus prompting an investigation as to the effect of THR-123 on the expression of several pro-inflammatory cytokines in a human renal tubular epithelial cell line (HK-2 cells). BMP7 and THR-123 inhibited TNF-α induced IL-6 production in dose dependent manner (FIG. 47 A). THR-123 also inhibits TNF-α-induced IL-8 (FIG. 47 B) and ICAM-1 production (FIG. 47 C) in HK-2 cells, suggesting that similar to the function of BMP7, THR-123 exhibits anti-inflammatory properties.

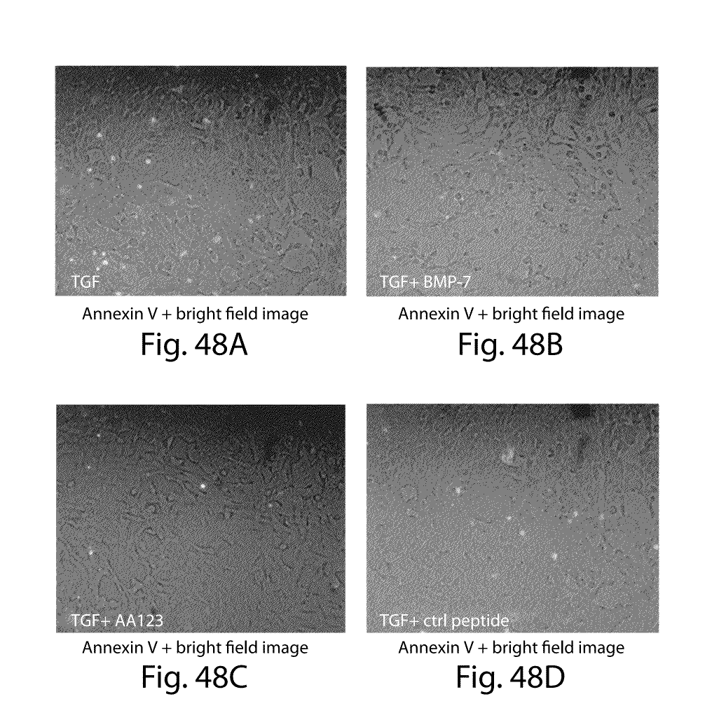

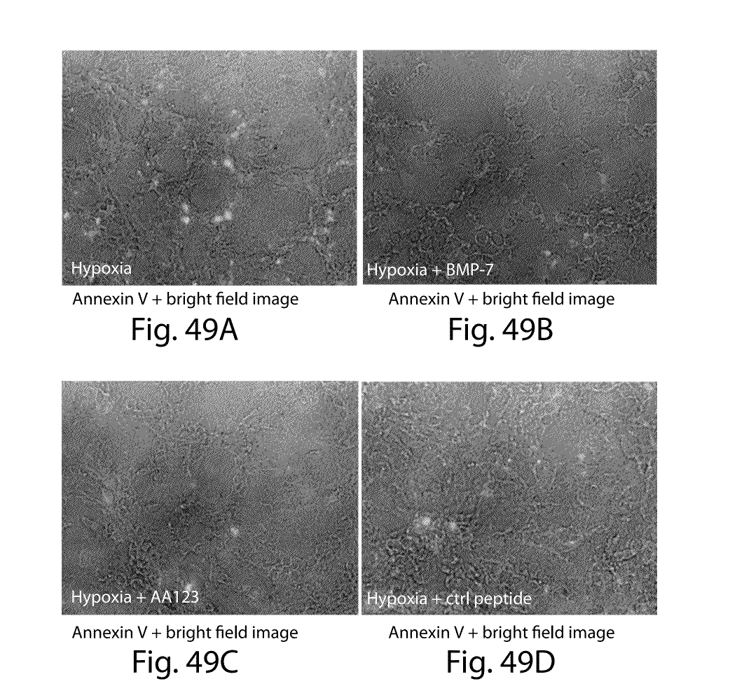

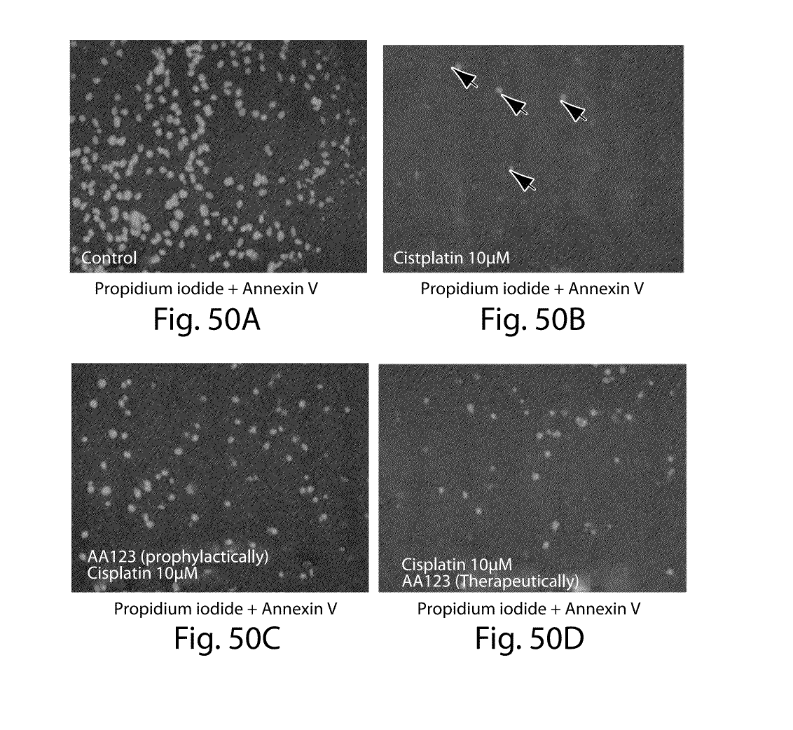

BMP-7 is also reported to protect tubular epithelial cells (TECs) from apoptosis²². TGF-β-induced apoptosis in TECs was analyzed by annexin V labeling. (FIG. 48 A). BMP7 and THR-123 exhibited similar anti-apoptotic activity (FIG. 48 B,C), while such anti-apoptotic activity was not detected when a control scrambled cyclic peptide was used (FIG. 48 C,D). Hypoxia induced apoptosis of TECs was also inhibited by BMP7 and THR-123 (FIG. 49 A-D). Additionally, cisplatin induced apoptosis was inhibited by THR-123 (FIG. 50 A-D).

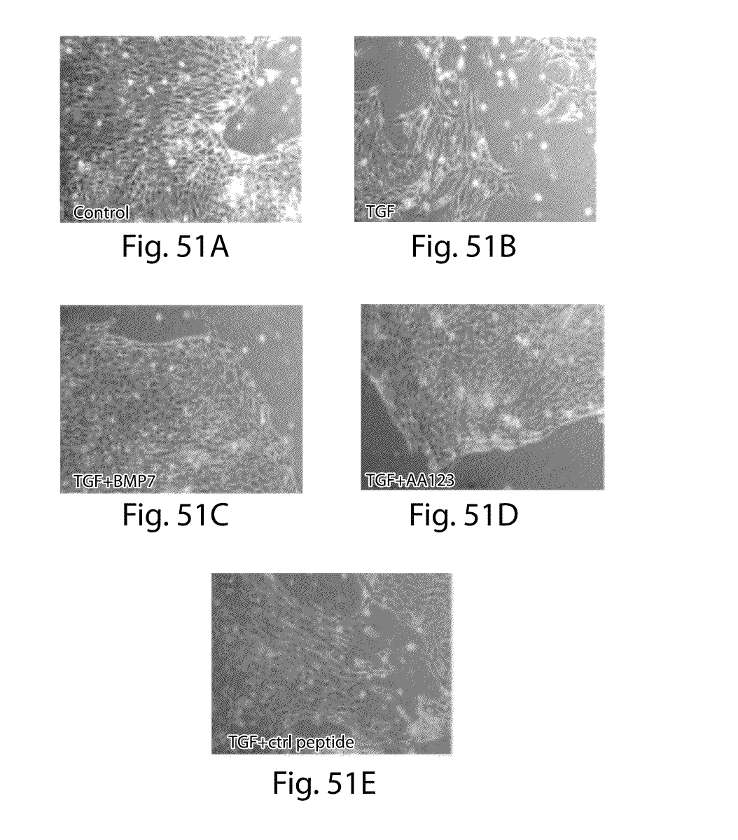

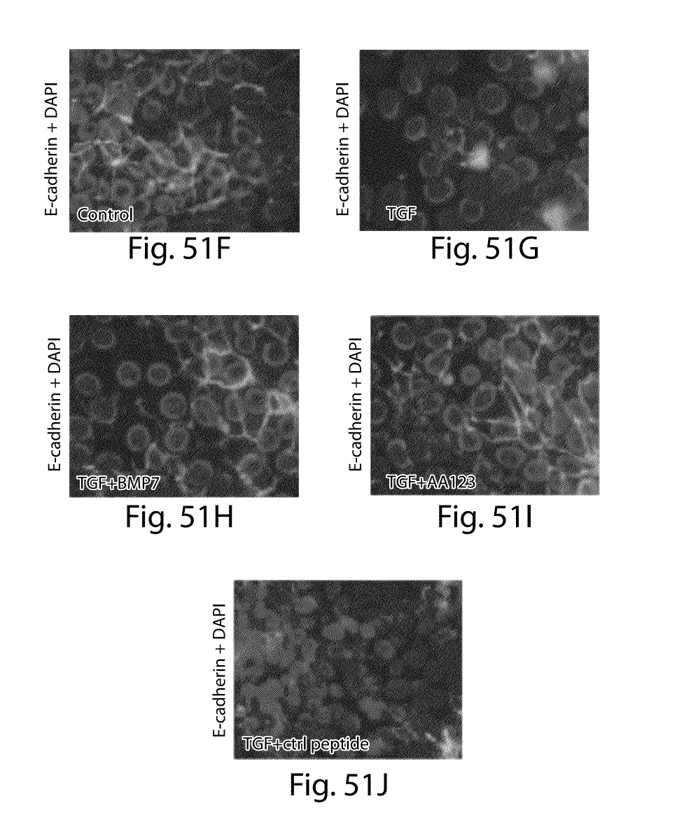

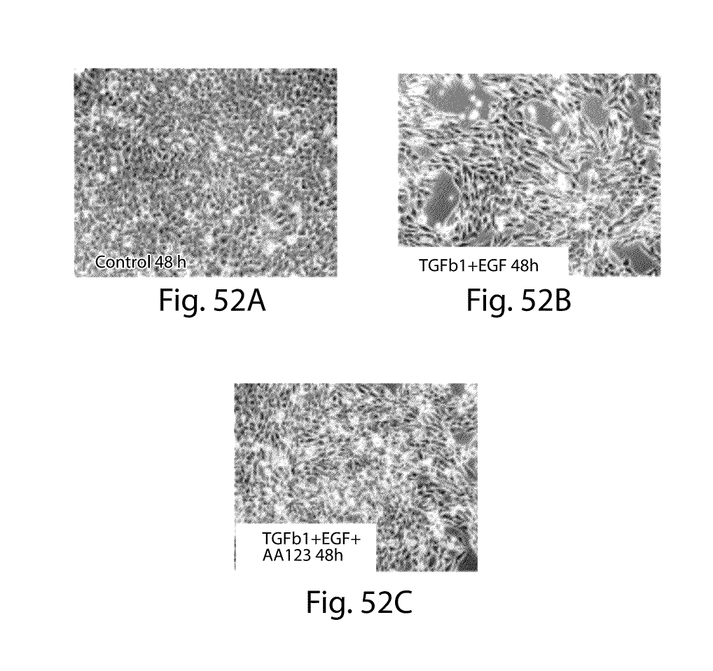

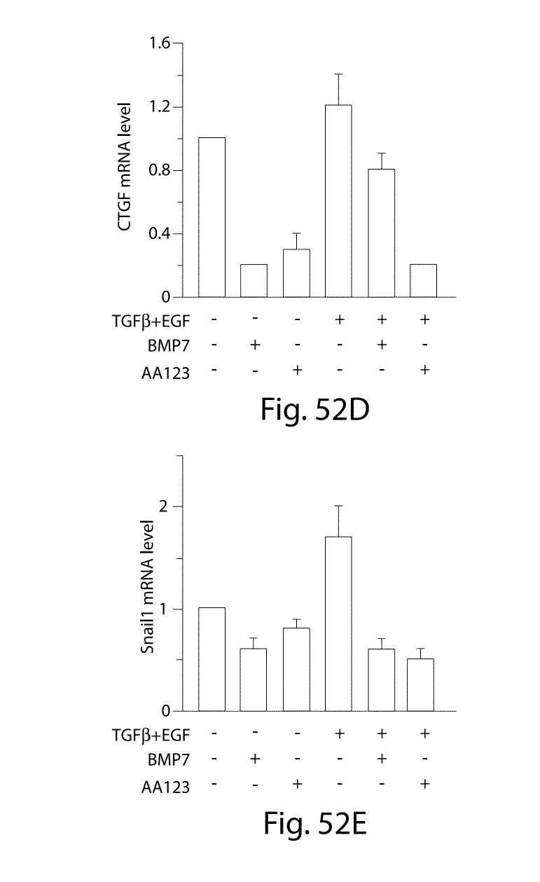

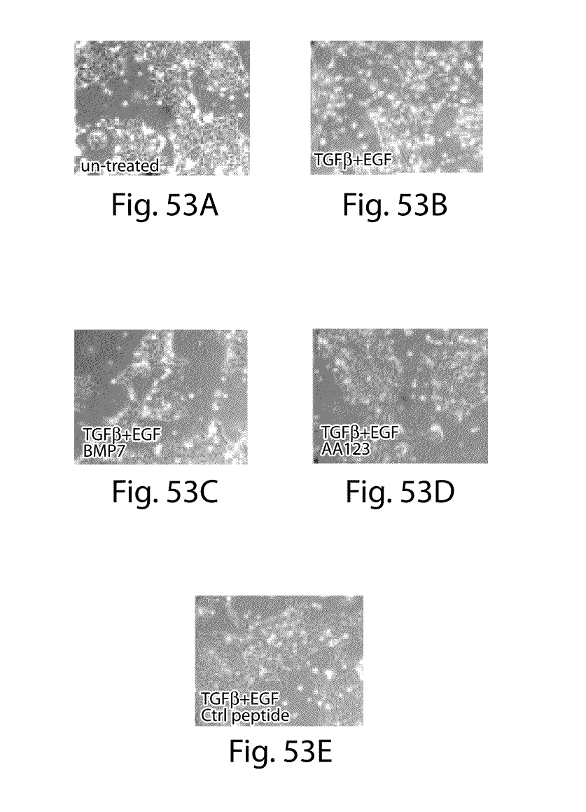

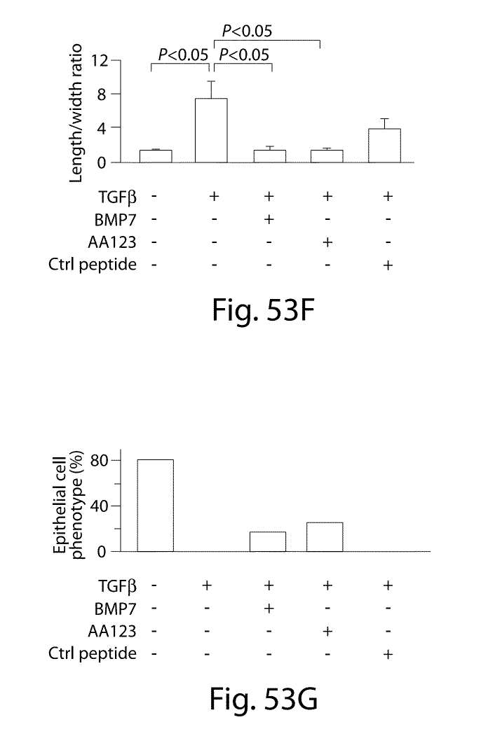

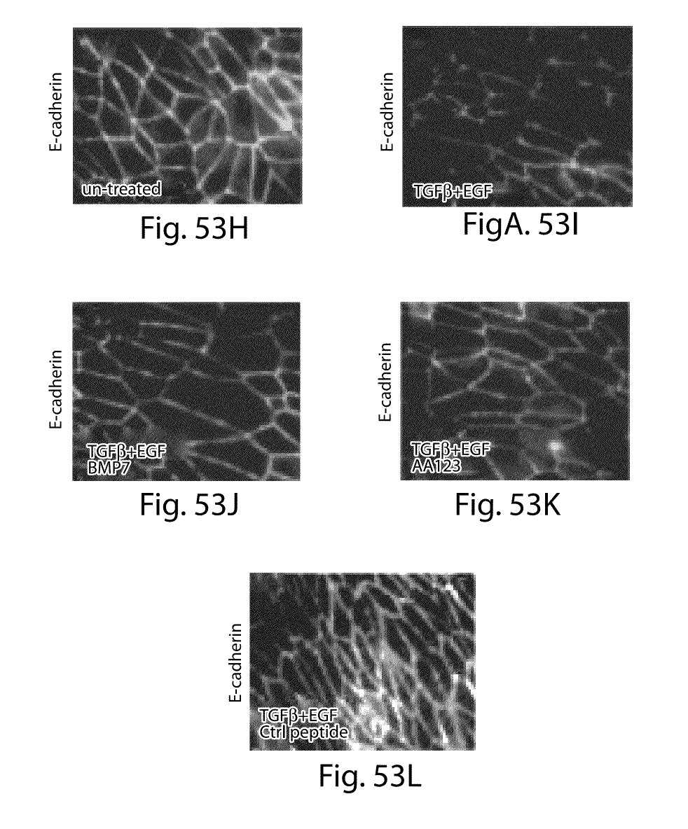

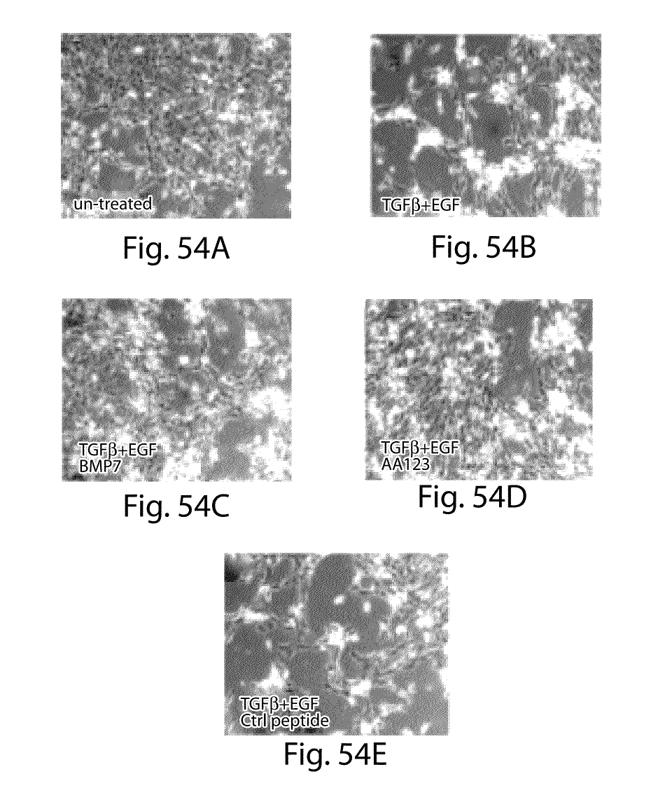

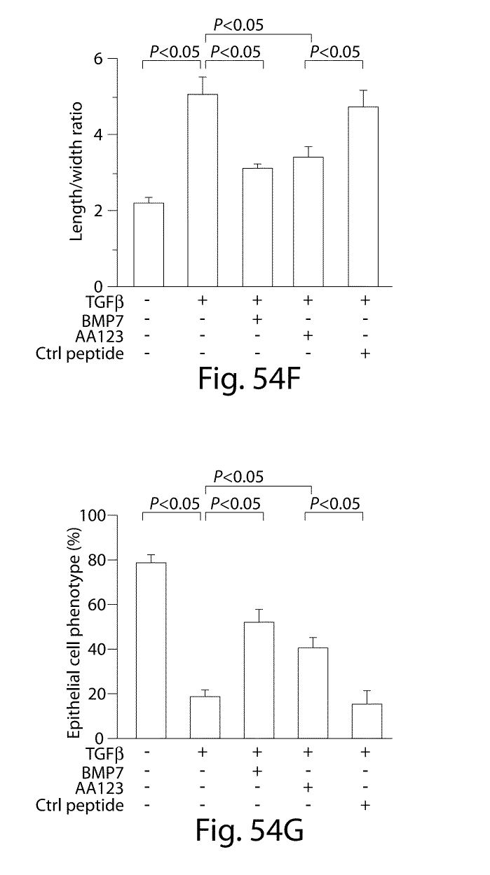

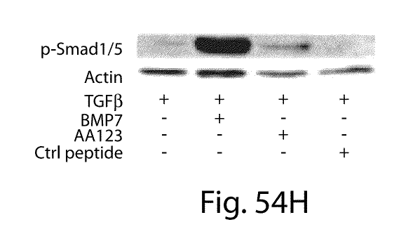

BMP7 has been shown to inhibit TGF-β induced epithelial-mesenchymal transition (EMT) program². Similar to BMP7, THR-123 inhibited TGF-β induced EMT program (FIG. 51 A-D, FIG. 52 A-C). TGF-β inhibited E-cadherin expression (FIG. 51 F and G), while both BMP7 and THR-123 restored TGF-β-suppressed E-cadherin to normal levels (FIGS. 51 H and I). Control cyclic scrambled peptide exhibited insignificant effect on the EMT program (FIG. 51 J). TGF-β induced expression of genes associated with EMT program such as snail and CTGF was inhibited by THR-123 (FIG. 52 D, E). After 48 hours of incubation with TGF-β and epidermal growth factor (EGF), renal epithelial cells exhibit an EMT program (FIG. 53 A, B, F, G and FIG. 54 A, B, F, G). The TGF-β-induced EMT in these cells was reversed by the treatment with BMP7 or THR-123 (FIG. 53 C, D, F, G and FIG. 54 C, D, F, G). Control peptide reveals insignificant effect on the EMT (FIG. 53 E and FIG. 54 E). THR-123-induced reversal of EMT was associated with restoration of E-cadherin expression (FIG. 53 H-L) and Smad1/5 phosphorylation (FIG. 54 H).

(E) THR-123 Protects Kidneys from Acute and Chronic Renal Injury and Fibrosis

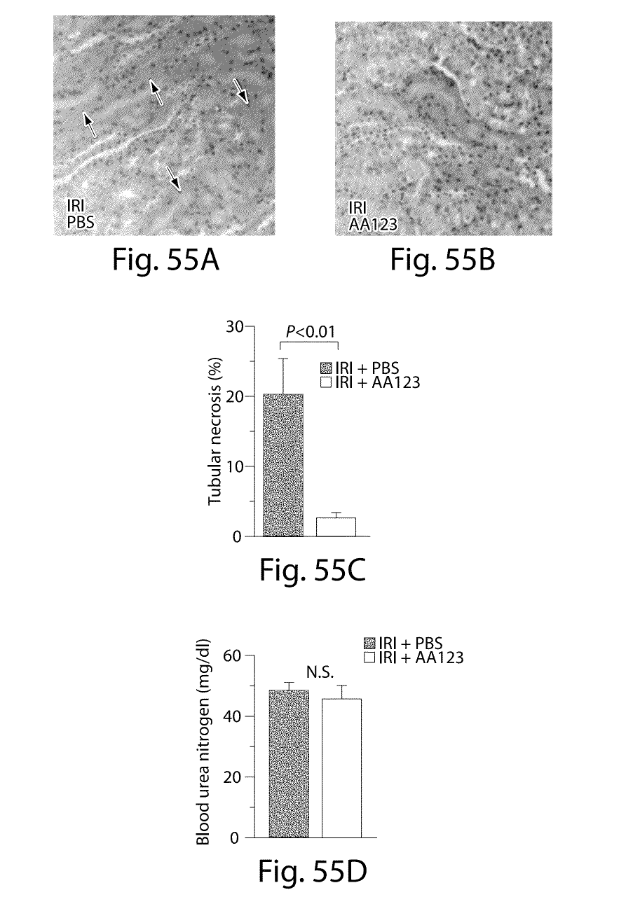

The effect of THR-123 on acute renal injury was analyzed using the ischemic re-perfusion injury (IRI) model in mice. Seven days after IRI, control mice exhibited renal morphology consistent with acute renal tubular necrosis characterized by tubular dilatation and flattened epithelial cells with eosinophilic homogenous cytoplasm (FIG. 55 A, C). THR-123-treated mice displayed significantly less tubular damage in IRI kidney when compared to control mice (FIG. 55 A-C). Blood urea nitrogen levels were similar in both groups (FIG. 55 D).

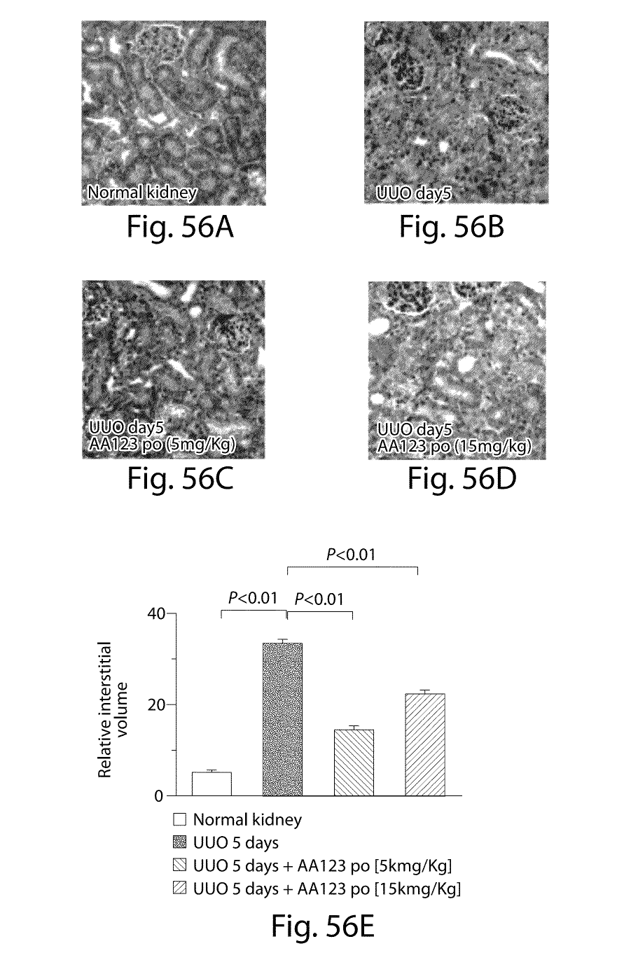

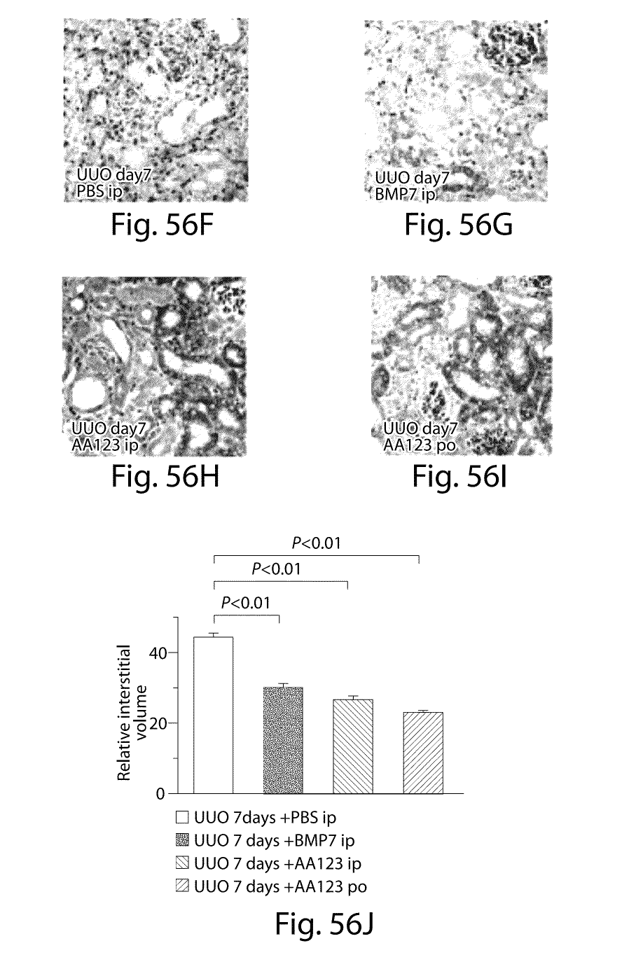

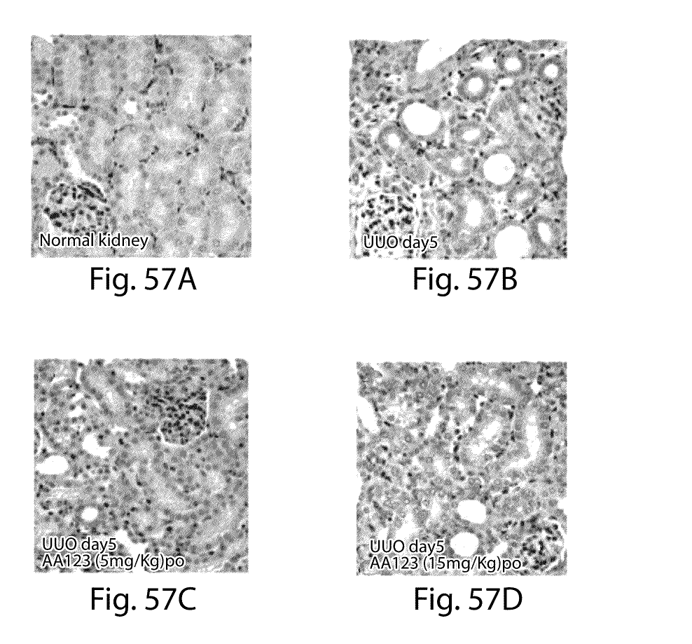

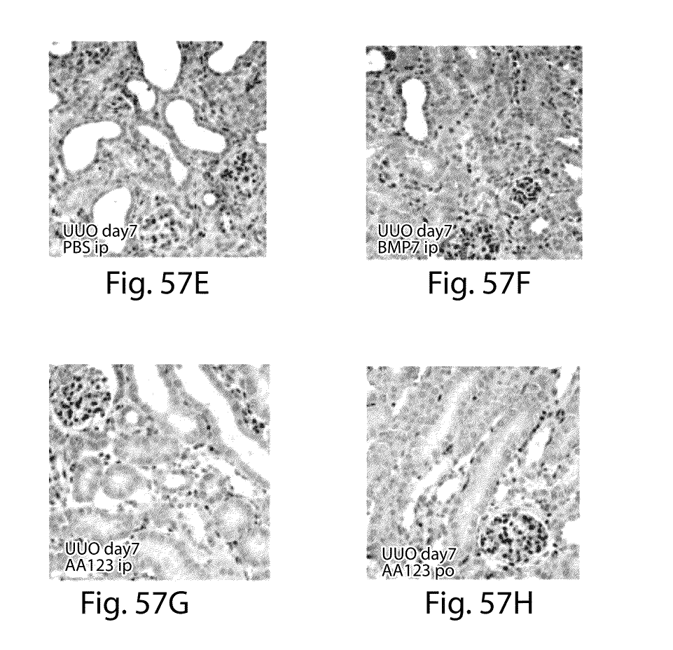

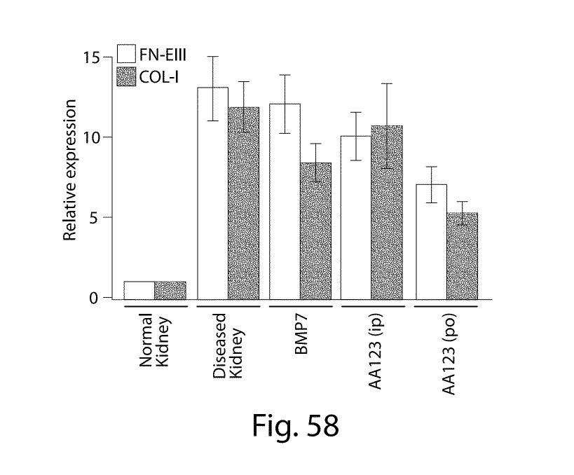

Unilateral ureteral obstruction (UUO) is a well-established model of severe renal interstitial injury and fibrosis (FIG. 56, FIG. 57). Five days after UUO, kidneys display significantly increased interstitial volume when compared to normal kidney (FIG. 56 A, B, E, and FIG. 57 A, B). Oral administration of THR-123 (5 mg/Kg or 15 mg/Kg) inhibited interstitial volume expansion in UUO kidneys when compared to untreated mice (FIG. 56 B-E and FIG. 57 C, D). Seven days after UUO, kidneys exhibited severe fibrosis with increased interstitial volume (FIG. 56 F, J and FIG. 57 E). Intraperitoneal administration of BMP-7 ameliorated interstitial volume expansion when compared to control mice (FIG. 56 F, G, J and FIG. 57 E, F). Both intraperitoneal and oral administration of THR-123 inhibited fibrosis (FIG. 56 H-J and FIG. 57 G, H). Decreased tubular damage with THR-123 treatment was associated with decreased expression of matrix components such as fibronectin and type I collagen (FIG. 58).

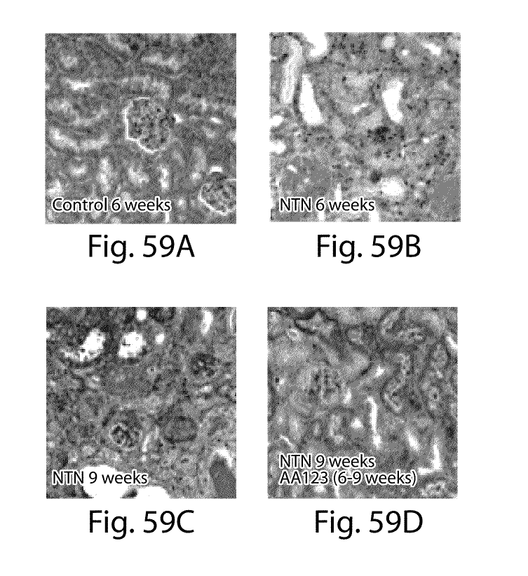

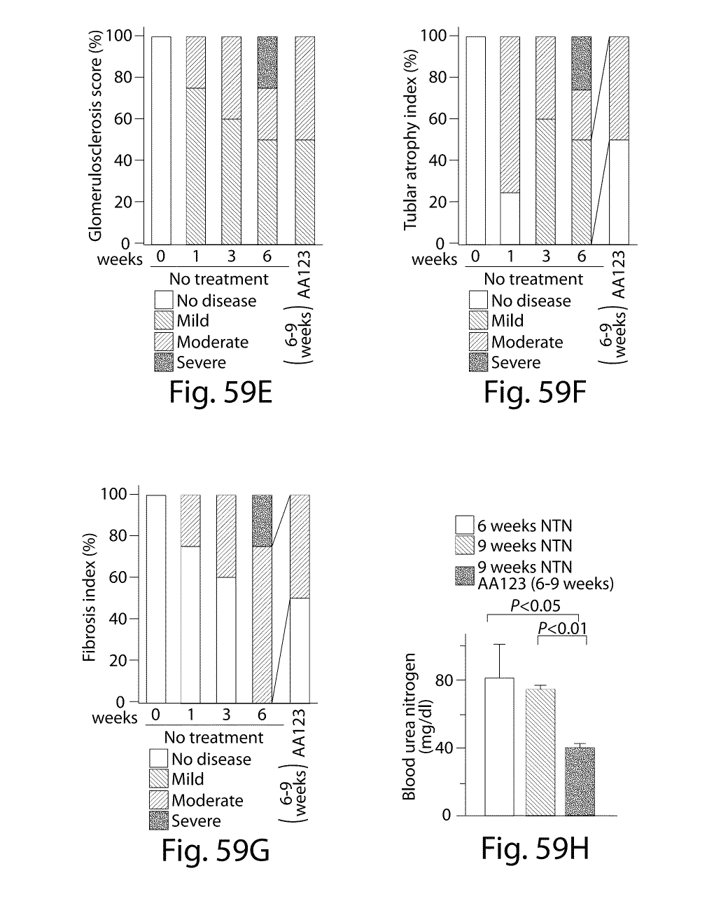

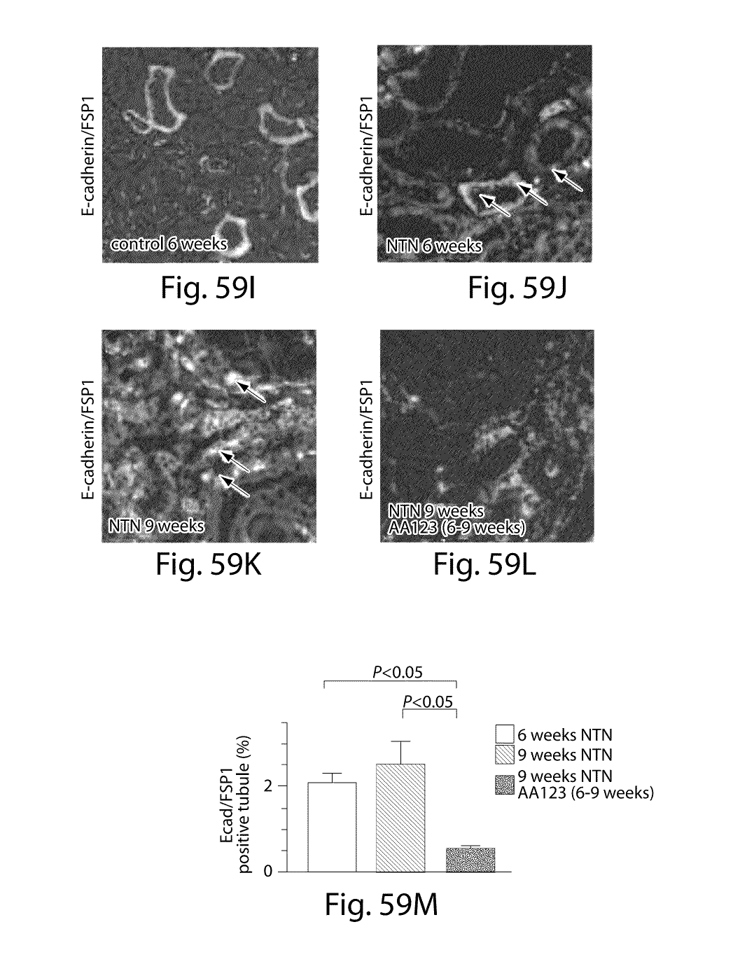

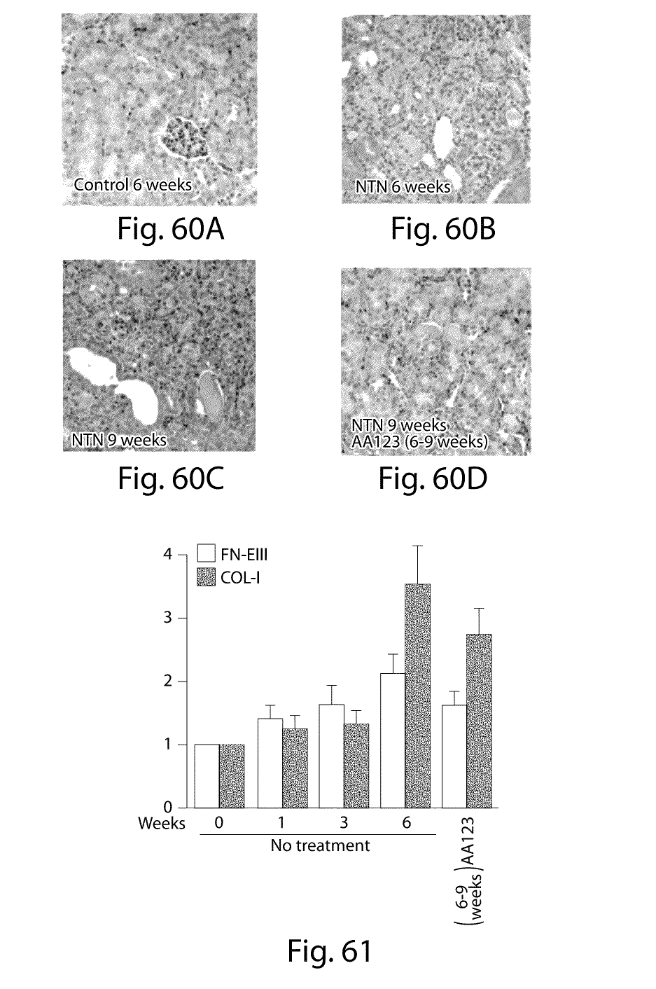

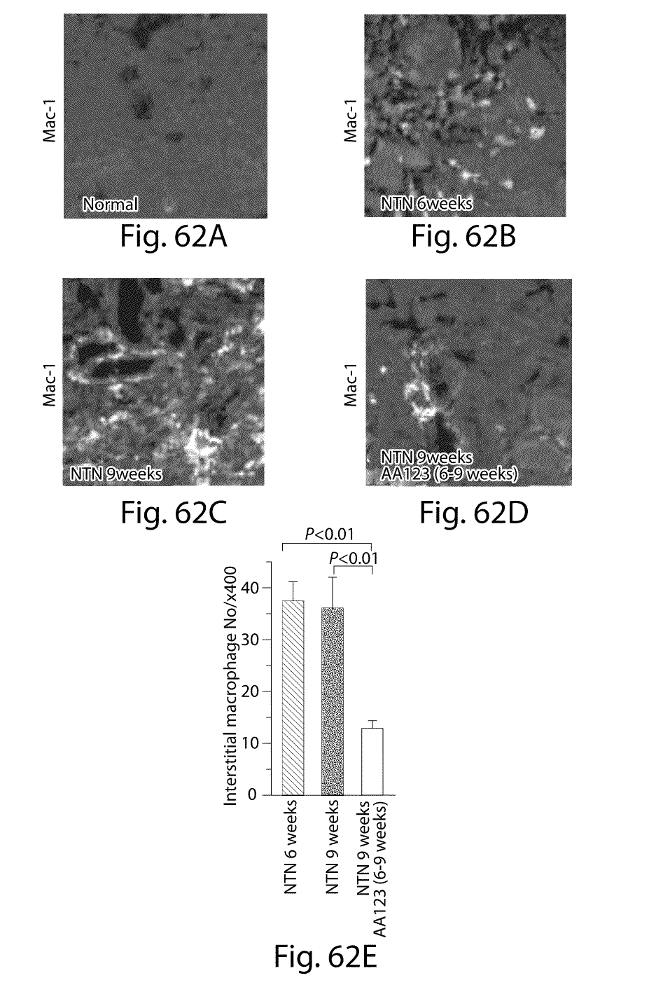

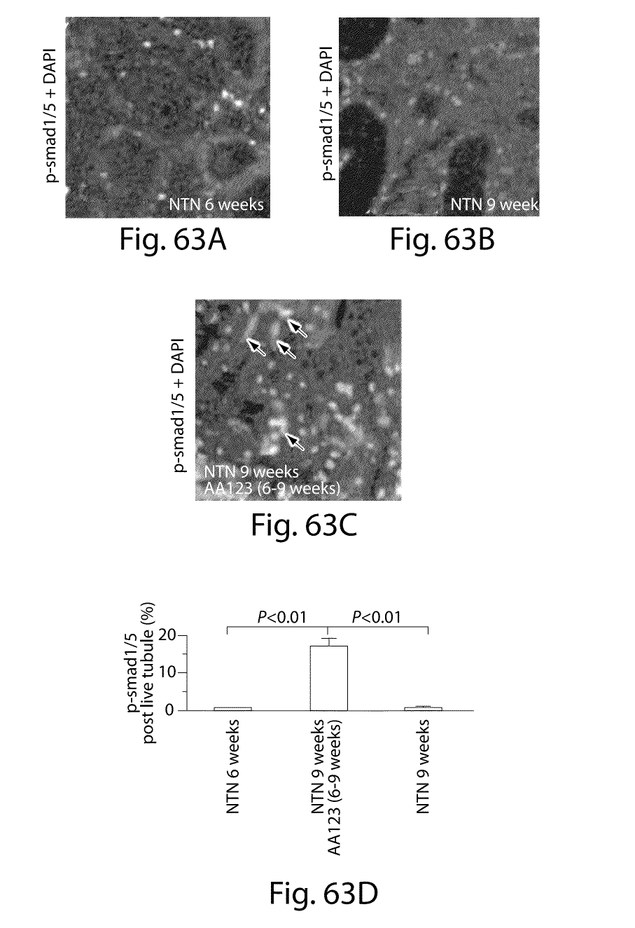

Next, the effect of THR-123 on the nephrotoxic nephritis induced by sheep anti-glomerular antibodies (NTN) model was analyzed. Kidneys with NTN exhibit severe crescentic glomerulonephritis with interstitial damage and fibrosis^{2,34}. Such lesions develop in a progressive manner in the CD-1 mice (FIGS. 59 A-C and E-G and FIG. 60 A-C). Six weeks following NTN induction, mice exhibited severe crescentic glomerulonephritis with severe interstitial damage and fibrosis (FIG. 59 B and FIG. 60 B). THR-123 treatment (initiated at 6 weeks following NTN induction) improved glomerular lesion (sclerosis) and tubular atrophy and fibrosis (FIG. 59 D-G and FIG. 60 D), associated with decreased expression of matrix components such as fibronectin and type I collagen (FIG. 61). Blood urea nitrogen was decreased after THR-123 treatment (FIG. 59 H). We identified tubular cells with EMT program, as being positive for both fibroblast specific protein (FSP)-1 and E-cadherin. Similar to previous reports², EMT program was evident in NTN kidneys when compared to normal kidney (FIG. 59 I-K, M). THR-123 treatment significantly decreased the number of cells exhibiting an EMT program (FIG. 59 L, M). NTN kidneys exhibited increased Mac-1 positive macrophages accumulation when compared to control normal kidney; and THR-123 treatment inhibited accumulation of macrophage (FIG. 62). THR-123 treated kidneys present with increased accumulation of phospho-Smad1/5, revealing a possible stimulation of Alk3-mediating pathway (FIG. 63).

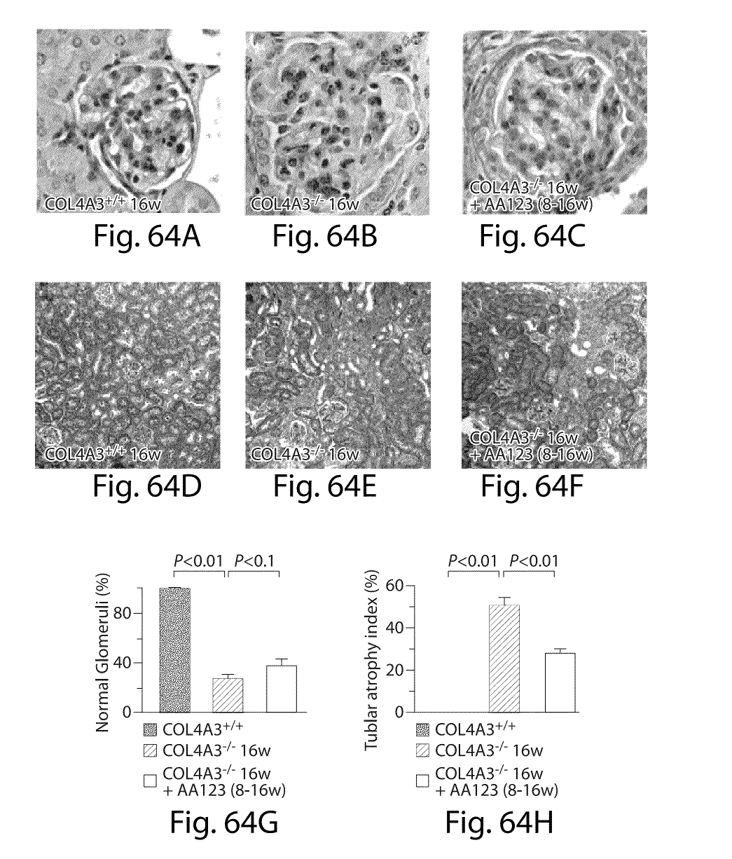

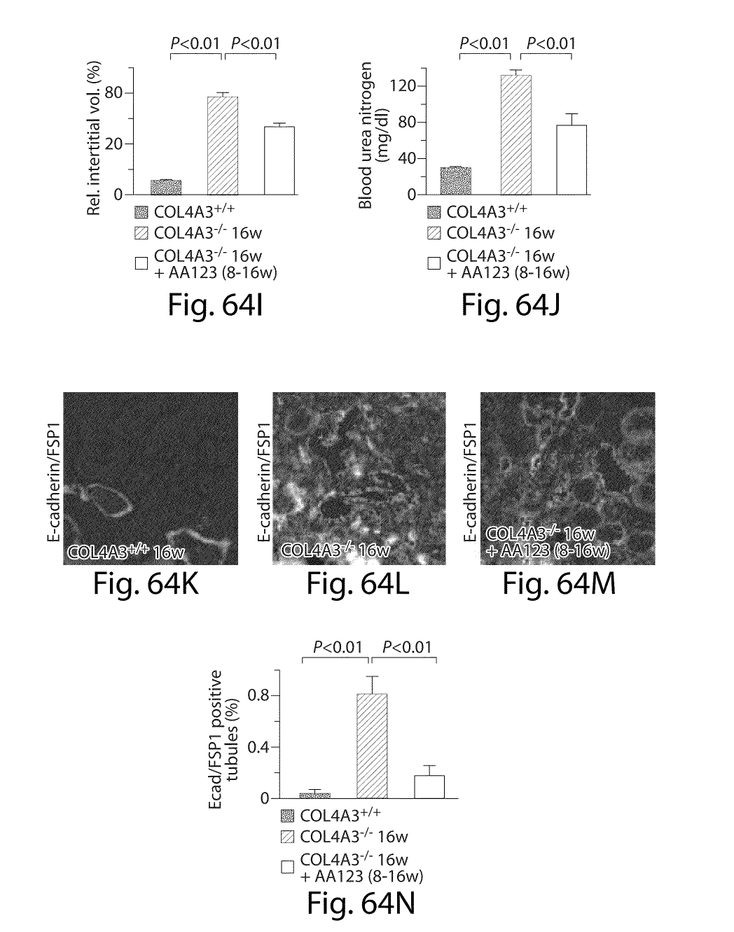

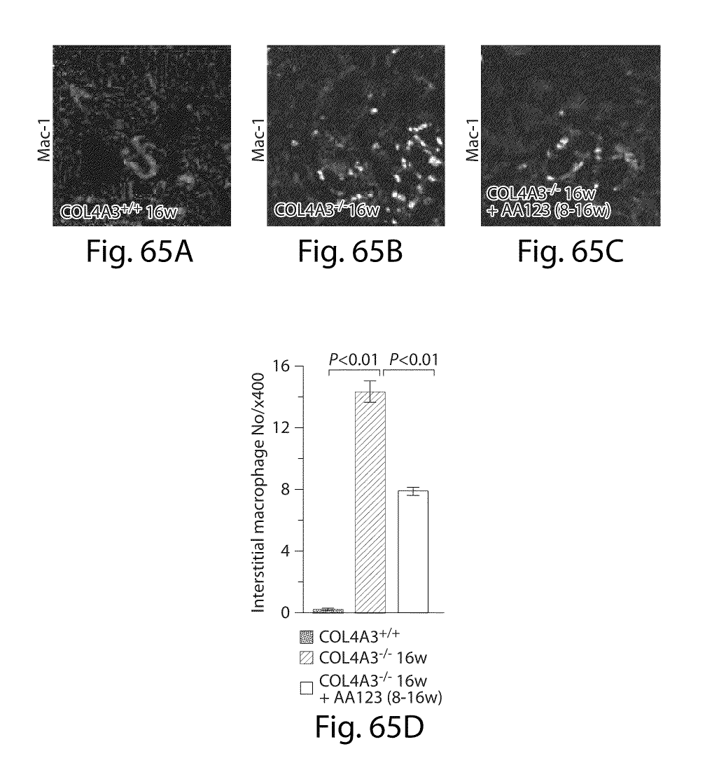

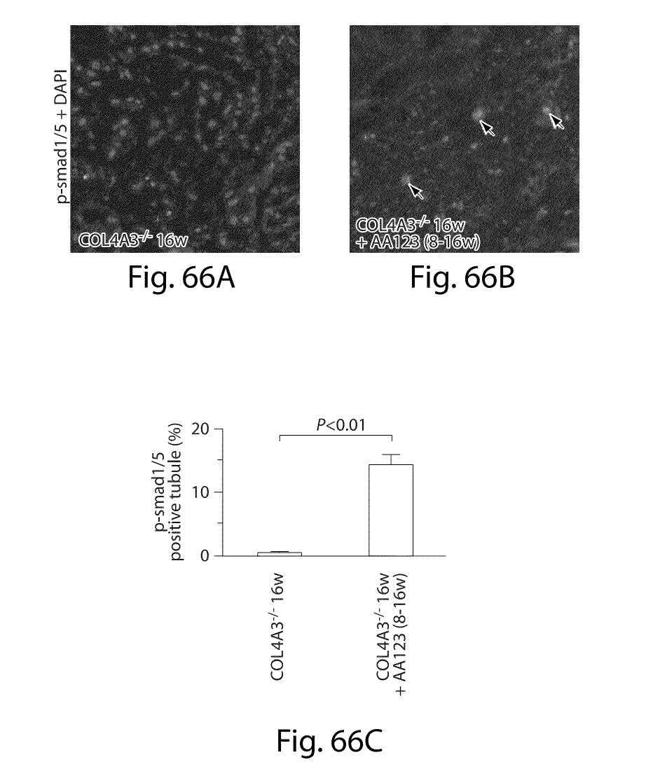

Alport syndrome is a inherited kidney disease caused by genetic mutations in genes encoding for type IV collagen proteins³⁵. The mice deficient in alpha3 chain of type IV collagen chain (COL4A3KO mice) mimic renal disease associated with Alport Syndrome. At 16 weeks of age, COL4A3KO mice exhibit increased glomerular abnormality, tubular atrophy and fibrosis when compared to wild-type kidney (FIG. 64 A-F). While THR-123 treatment did not alter glomerular abnormalities (FIG. 64 C, G), it significantly inhibited tubular atrophy and interstitial fibrosis (FIG. 64 F, H, I). Blood urea nitrogen levels are increased in COL4A3KO mice when compared to wild-type mice (FIG. 64 J). THR-123 significantly improved blood urea nitrogen level in COL4A3KO mice (FIG. 64 J). In COL4A3KO kidney, the number of cells exhibiting EMT program was significantly higher when compared to wild-type kidney (FIG. 64 K, L, N). THR-123 treatment inhibited such acquisition of an EMT program (FIG. 64 M, N). Macrophage infiltration in COL4A3KO kidney is increased when compared to control kidney and THR-123 treatment inhibited macrophage infiltration (FIG. 65), THR-123 treated COL4A3KO kidneys are associated with increased accumulation of phospho-smad1/5 (FIG. 66).

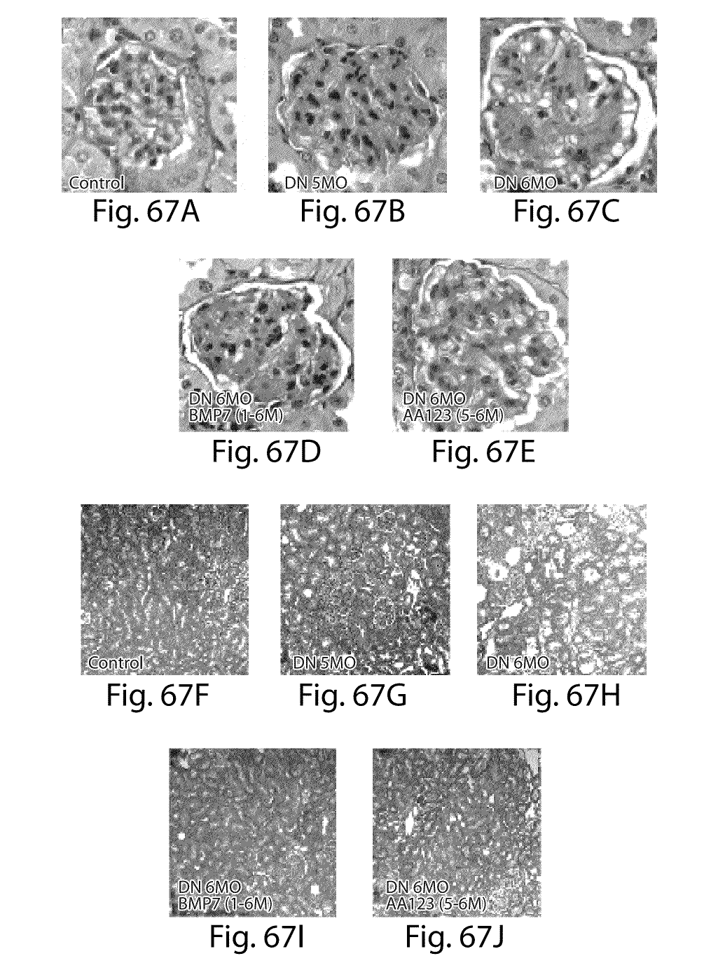

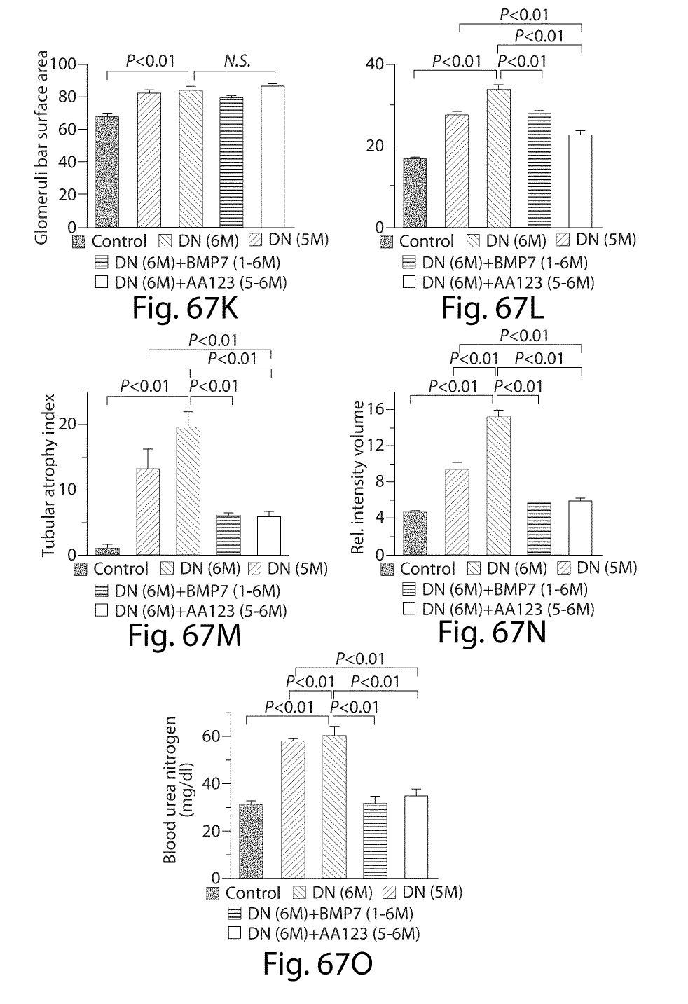

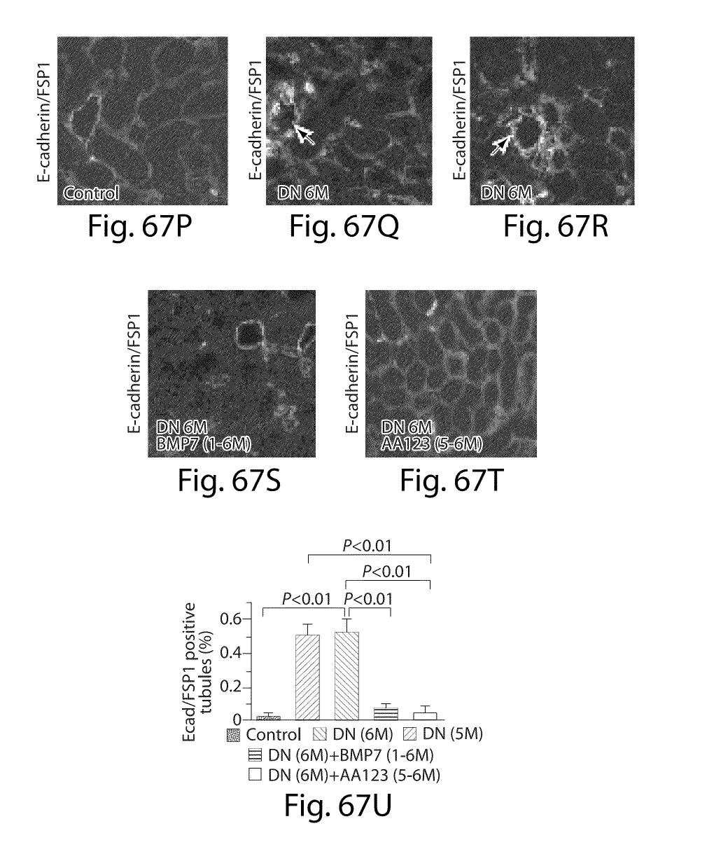

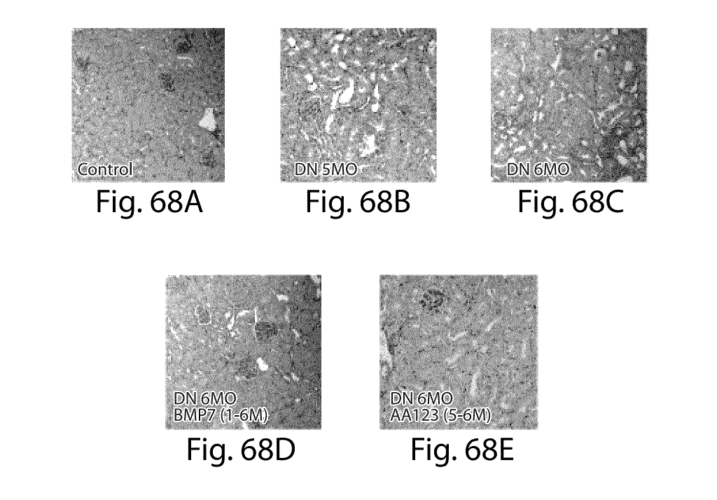

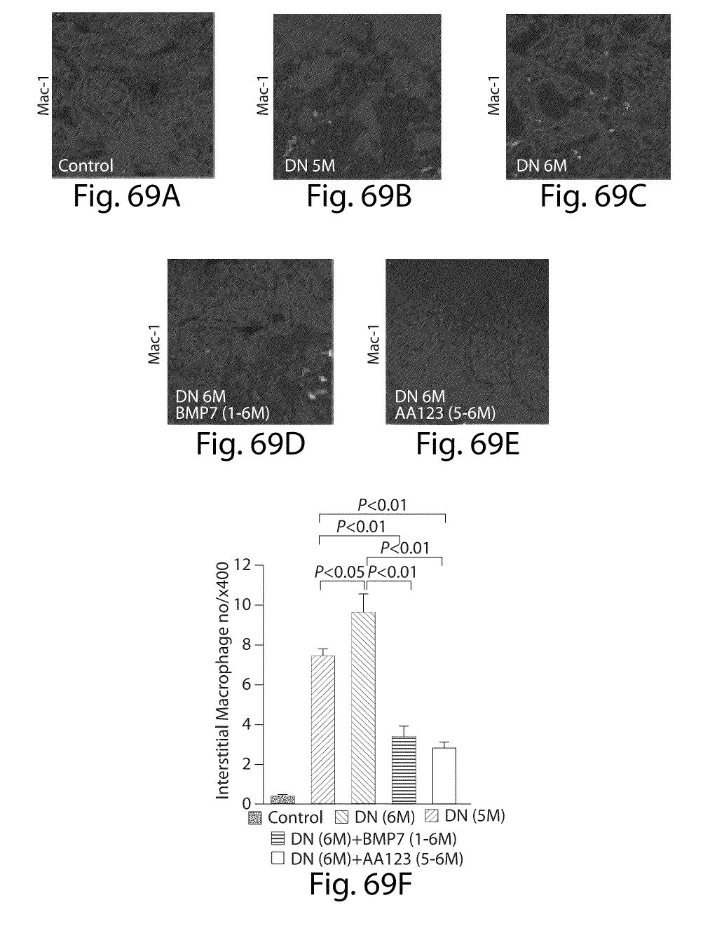

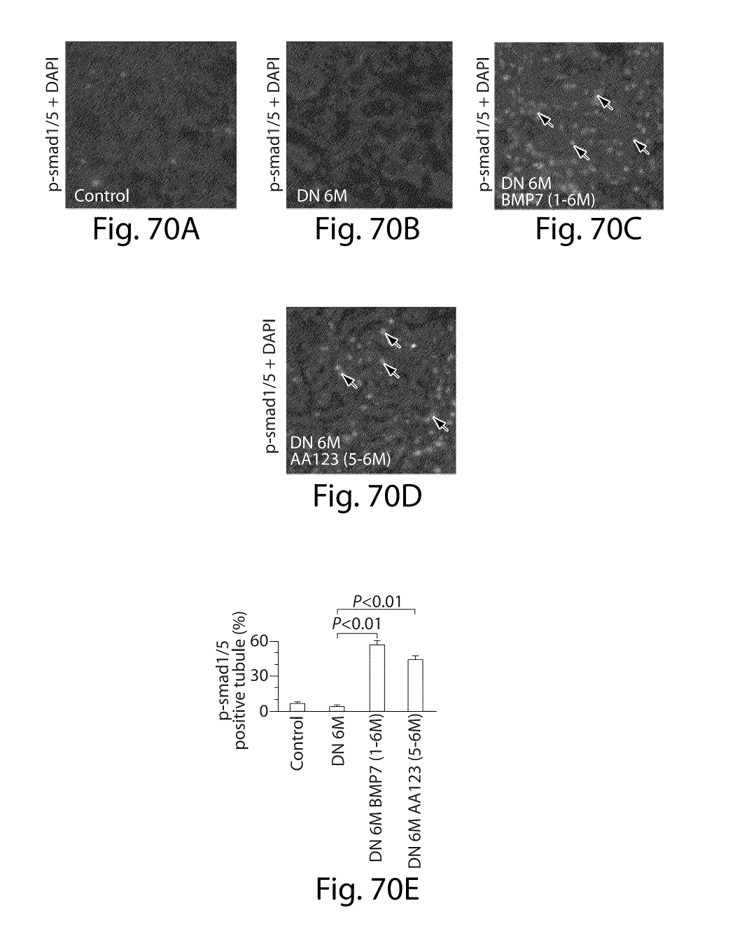

Next, the efficacy of THR-123 in controlling diabetic nephropathy (DN) in mice was evaluated. CD-1 mice injected with streptozotocin (STZ) exhibit increased glomerular mesangial matrix with increased glomerular surface area associated with interstitial damages by 6 months when compared to control mice, suggesting advanced DN (FIG. 67 A-C, F-H, FIG. 68 A-C). While neither BMP-7 nor THR-123 inhibited glomerular surface area increase in diabetic mice (FIG. 67 D, E, K), both BMP-7 and THR-123 inhibited mesangium expansion in STZ-induced 6 month DN (FIG. 67 B-E, L). Furthermore, THR-123 treatment (5-6 months) reversed mesangial matrix expansion when compared to mice with 5 months of DN (before THR-123 administration began) (FIG. 67 B, E, L). DN mice displayed increased tubular atrophy and interstitial volume when compared to control mice (FIG. 67 F-H, M, N, FIG. 68 A-C). Treatment with BMP-7 (1-6 months treatment) or THR-123 (5-6 months treatment) inhibited tubular atrophy and interstitial volume increase (FIG. 67 I, J, M, N, FIG. 68 D, E). THR-123 reversed tubular atrophy and interstitial volume expansion (FIG. 67 M, N). Blood urea nitrogen levels increased in DN when measured at 5 and 6 month following STZ administration (FIG. 67 O). Both BMP-7 and THR-123 reversed renal dysfunction in DN (FIG. 67 O). Both BMP-7 and THR-123 treatment inhibited EMT program (FIG. 67 P-R, S-U), and macrophage infiltration (FIG. 69). THR-123 treated kidneys also are associated with increased accumulation of phospho-Smad1/5 (FIG. 70).

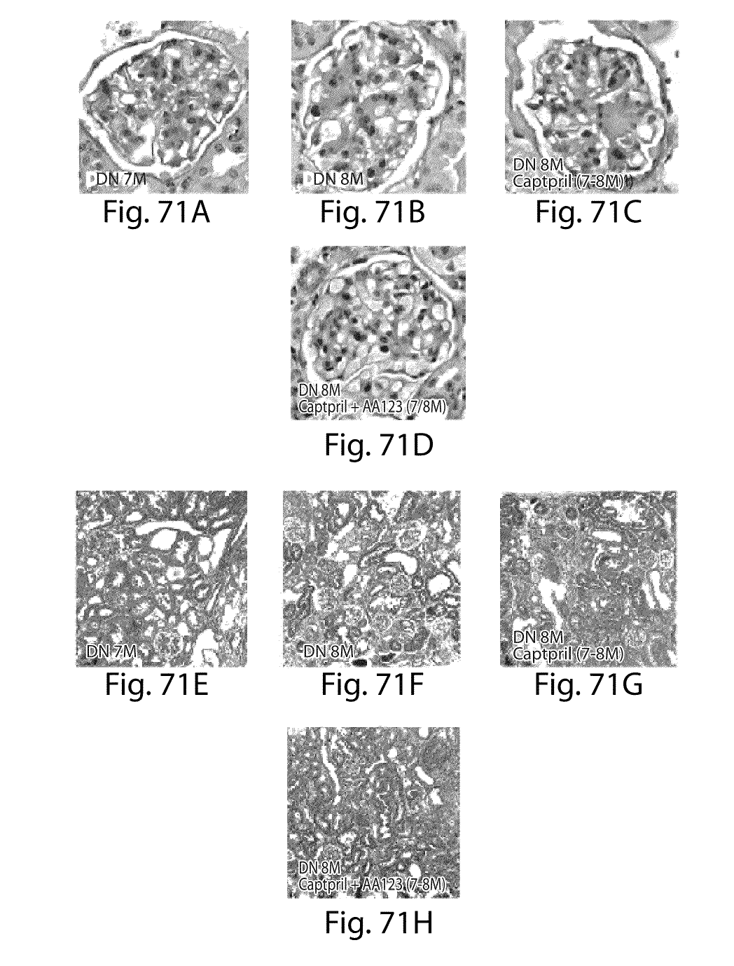

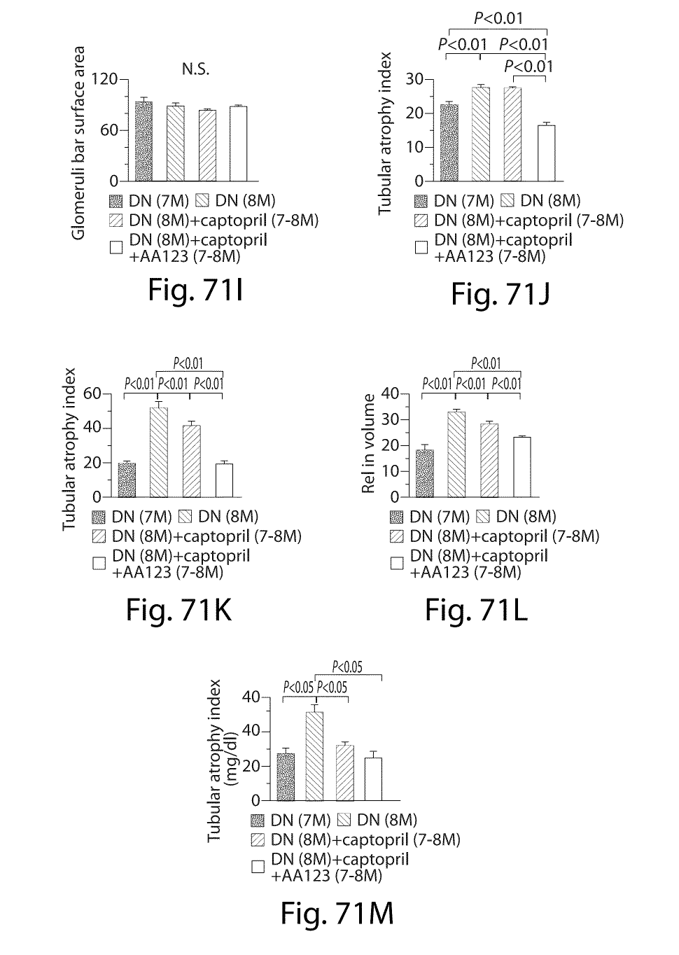

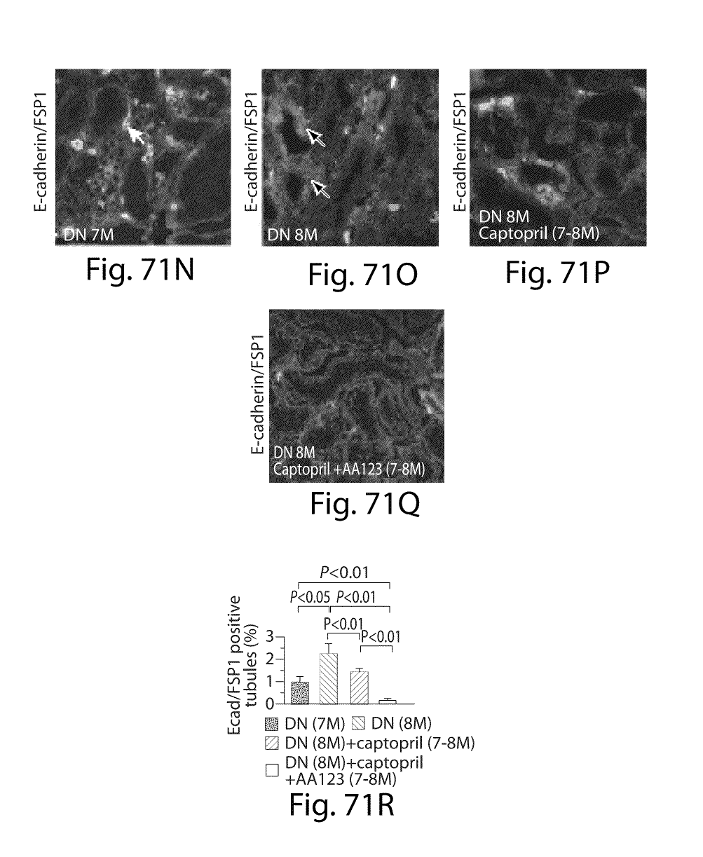

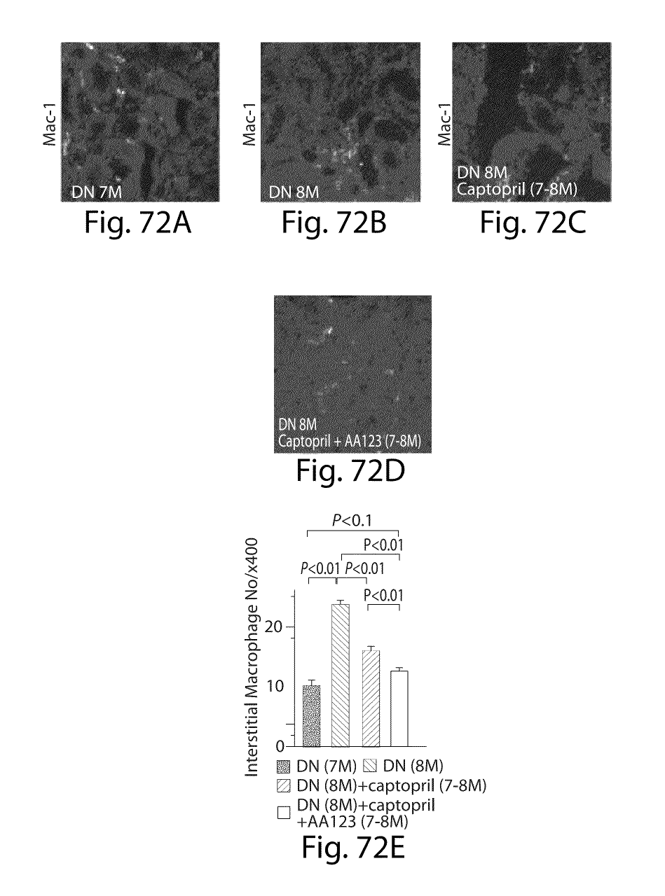

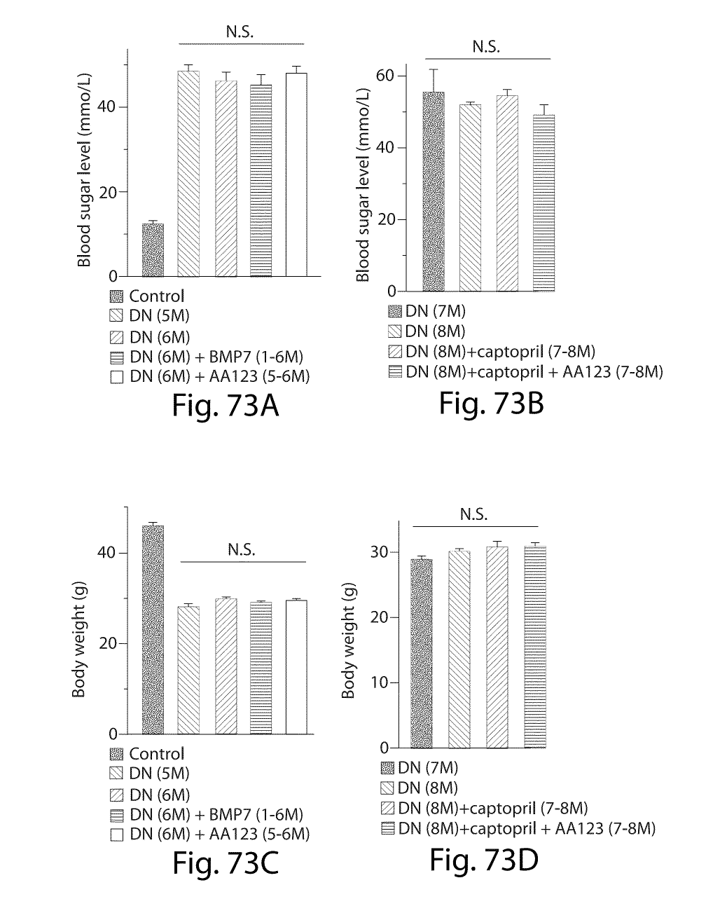

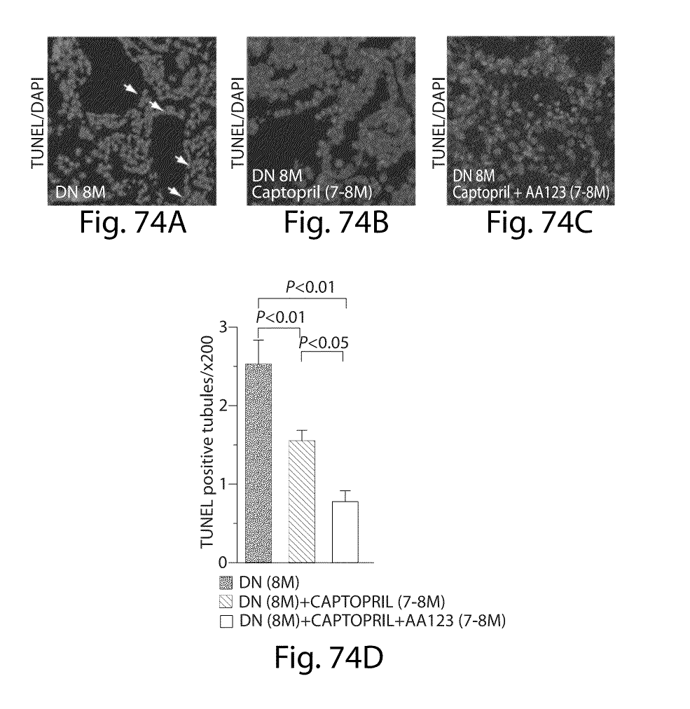

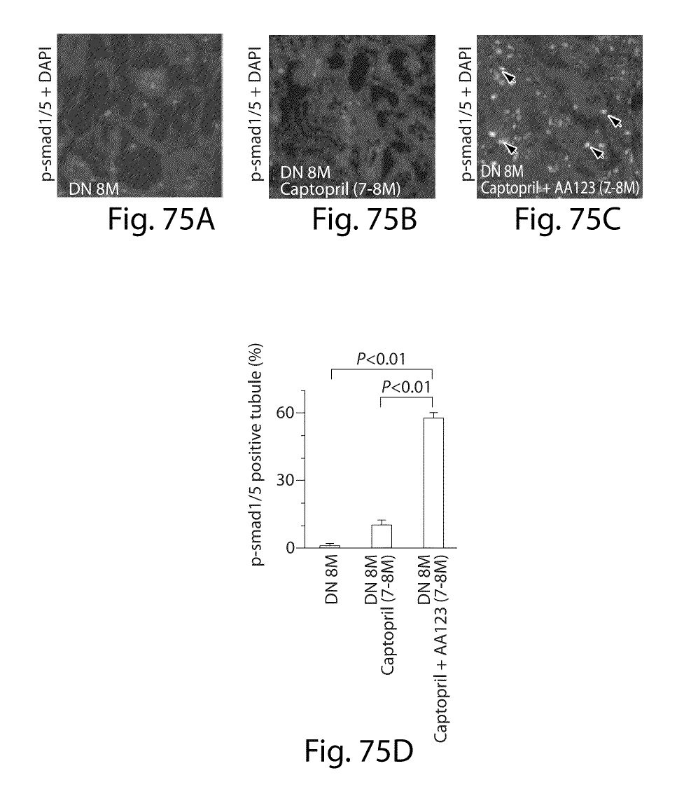

Angiotensin-converting enzyme inhibitor (ACE-I) is a well-established drug used to control progression of several chronic progressive kidney diseases including diabetic nephropathy^{36,37}. Therefore, we tested THR-123 and ACE-I [captopril (CPR)] in combination in mice with advanced diabetic kidney disease associated fibrosis. Seven months after induction of diabetes, DN kidneys display a significant increase in glomerular surface area and mesangial matrix deposition (FIG. 71 A). CPR and CPR/THR-123 combination treatment was initiated in mice with severe DN at 7 months following DN induction (FIG. 71 A-H). Glomerular surface area remained identical in all groups analyzed (FIG. 71 A-D, I). CPR treatment did not inhibit progression of mesangial matrix expansion in these experiments (FIG. 71 A-C, J), but a combination of CPR with THR-123 significantly reduced mesangial expansion and reversed it when compared to untreated control mice (FIG. 71 D, J). Between 7 to 8 months (late-stage) after induction of diabetes, DN kidney exhibited tubular atrophy and interstitial volume expansion (FIG. 71 E, F, K, L). CPR alone partially inhibited tubulo-interstitial alterations in DN kidney while a combination of CPR with THR-123 completely inhibited tubular atrophy and interstitial volume expansion (FIG. 71 G, H, K, L). Blood urea nitrogen level analysis revealed that DN mice exhibit significant renal function deterioration between 7 and 8 months after induction of diabetes (FIG. 71 M). CPR did not (p=0.08), but combination therapy significantly inhibited, progressive loss of renal function (FIG. 71 M). EMT program was inhibited by CPR alone (FIG. 71 N-P, R) and also by the CPR/THR-123 combination treatment (FIG. 71 N, O, Q, R). Both CPR and CPR-THR-123 combination therapy inhibited macrophage infiltration (FIG. 27). Blood sugar level and body weight were not altered in all the groups analyzed when compared to untreated diabetic mice at similar age (FIG. 73). CPR significantly inhibited apoptosis in diabetic kidney (FIG. 74) and CPR-THR-123 combination therapy exhibited additive anti-apoptotic effects (FIG. 74). CPR-THR-123 treated kidneys were associated with increased accumulation of phospho-smad1/5 (FIG. 75).

(F) THR-123 does not Inhibit Renal Injury and Fibrosis in Mice with Tubular Epithelial Cell Specific Deficiency in Alk-3 Receptor

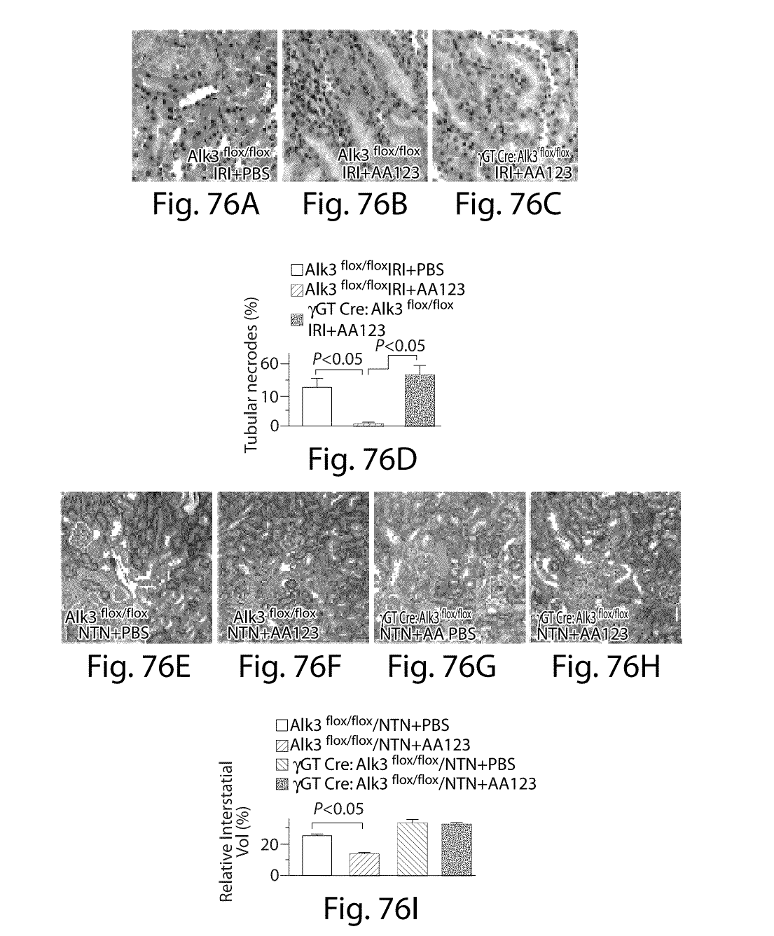

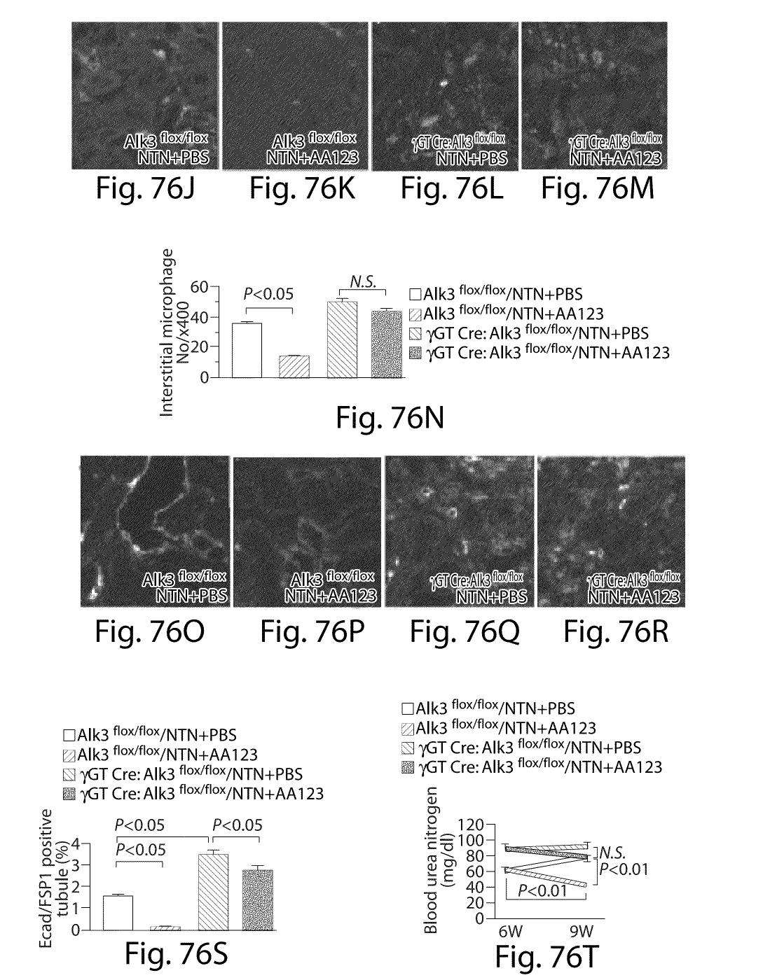

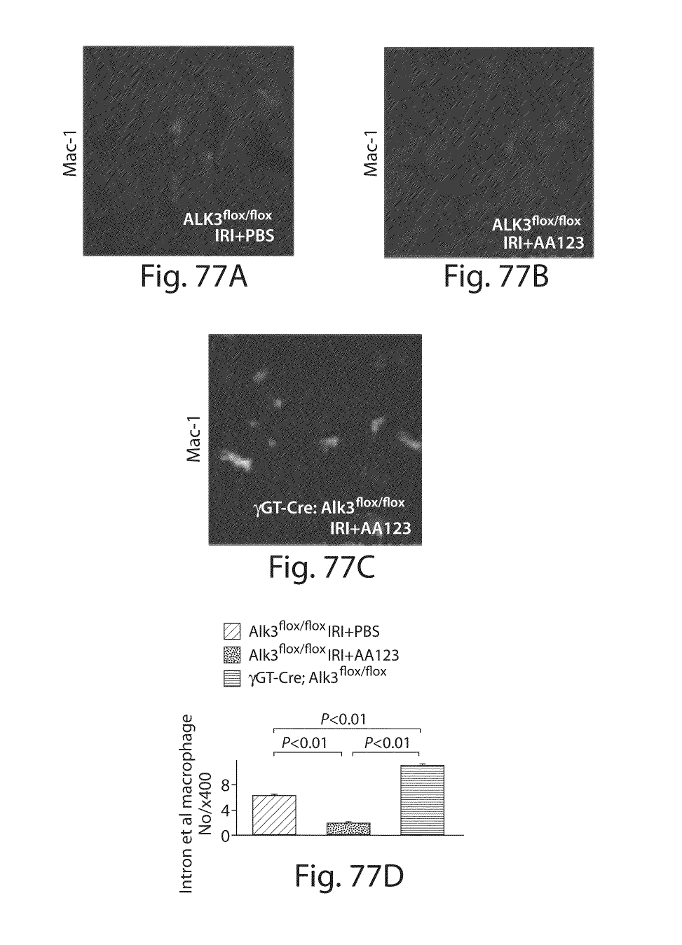

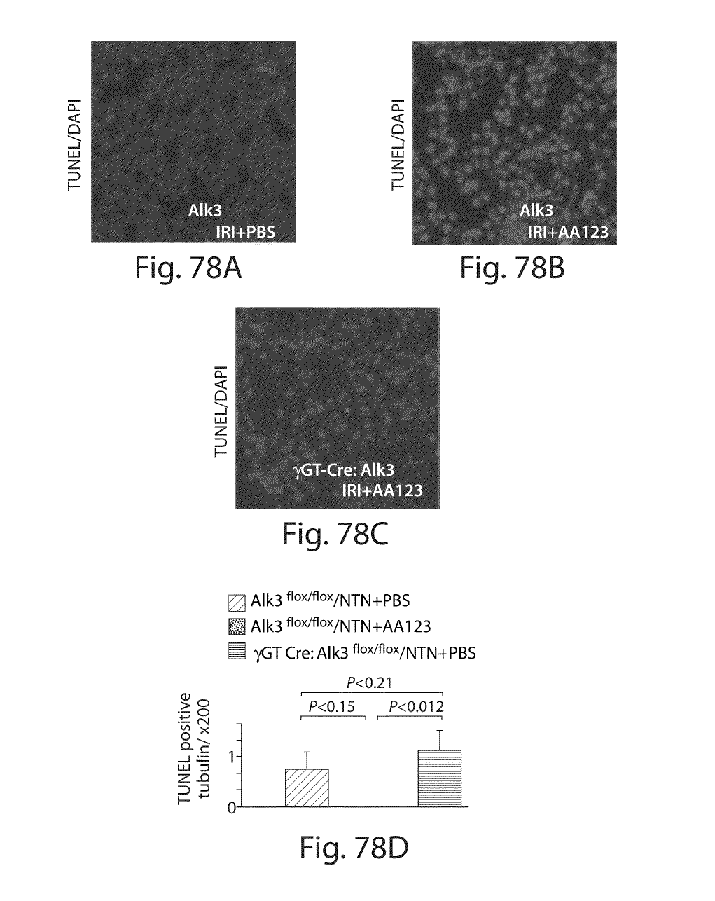

THR-123 binds to Alk-3 receptor and induced actions that mimic BMP7 (vide supra). All the above experiments suggest that THR-123 functions to suppress renal injury and fibrosis by inhibiting inflammation, apoptosis and EMT program. To functionally validate the target of THR-123 in exerting such reno-protective activity in mice, the efficacy of THR-123 in the Alk-3 deleted mice subjected to renal injury was tested (FIG. 42 K). The Alk-3 deleted mice and their littermate (control) mice were subjected to IRI. The mice with Alk-3 deficiency exhibit accelerated acute renal injury when compared to the control mice (FIG. 76 A-D). THR-123 inhibited renal injury in the control mice but does not exhibit therapeutic effect in the Alk-3 deleted mice (FIG. 76 A-D). Alk-3 dependent action of THR-123 in the control mice was associated with the reduction in macrophage accumulation (FIG. 77) and decreased tubular apoptosis (FIG. 78).

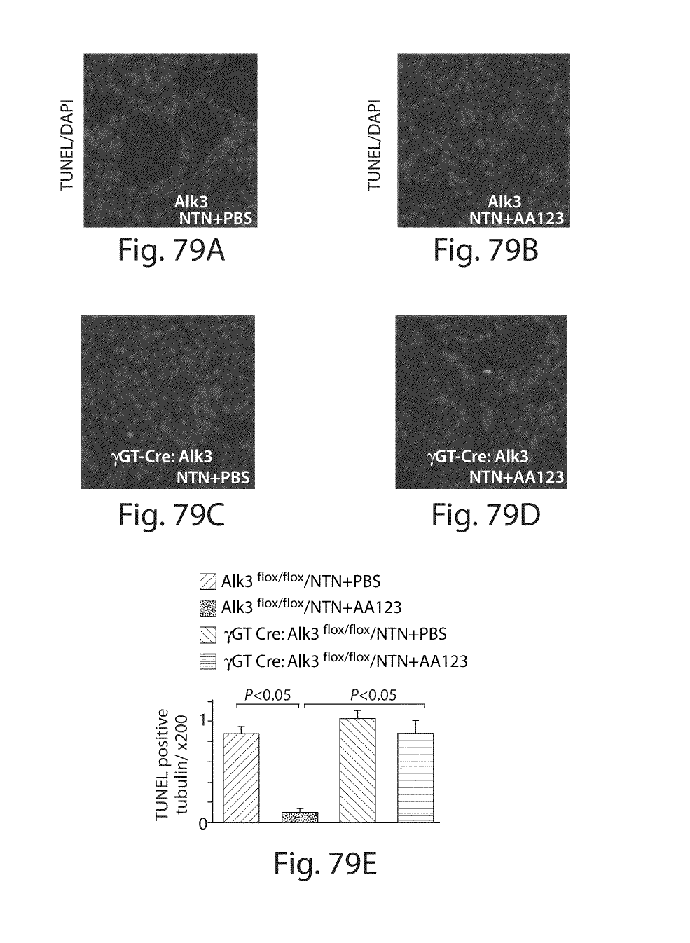

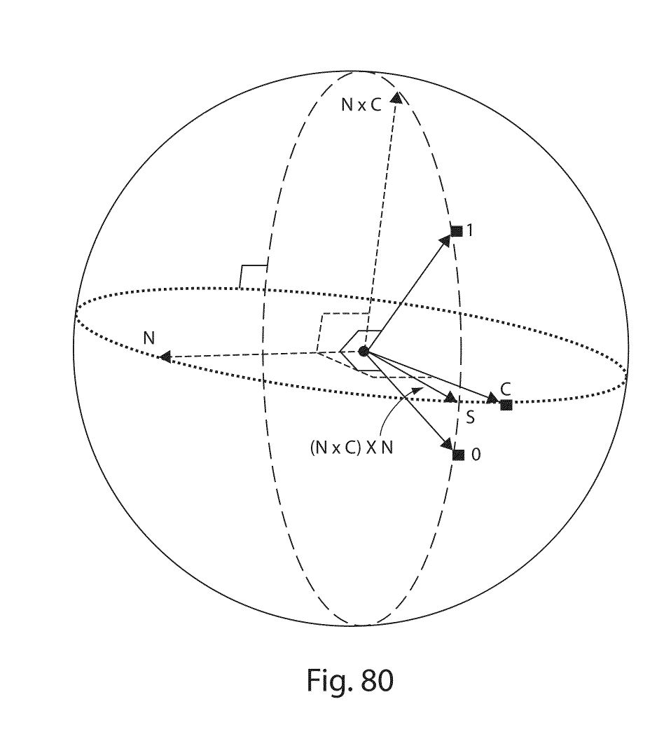

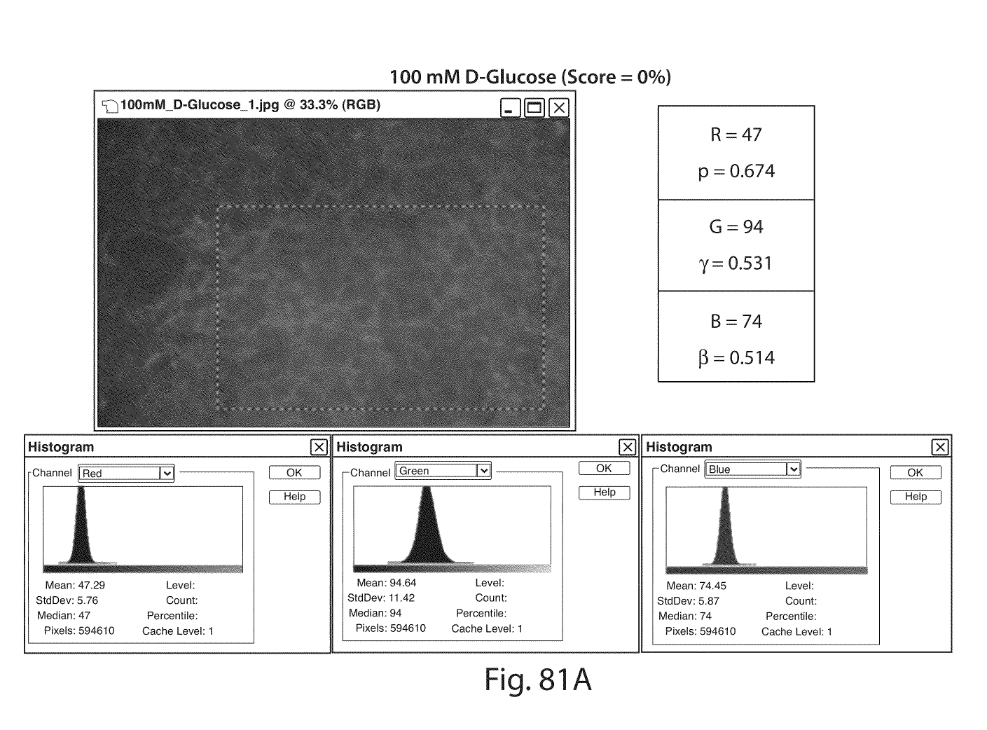

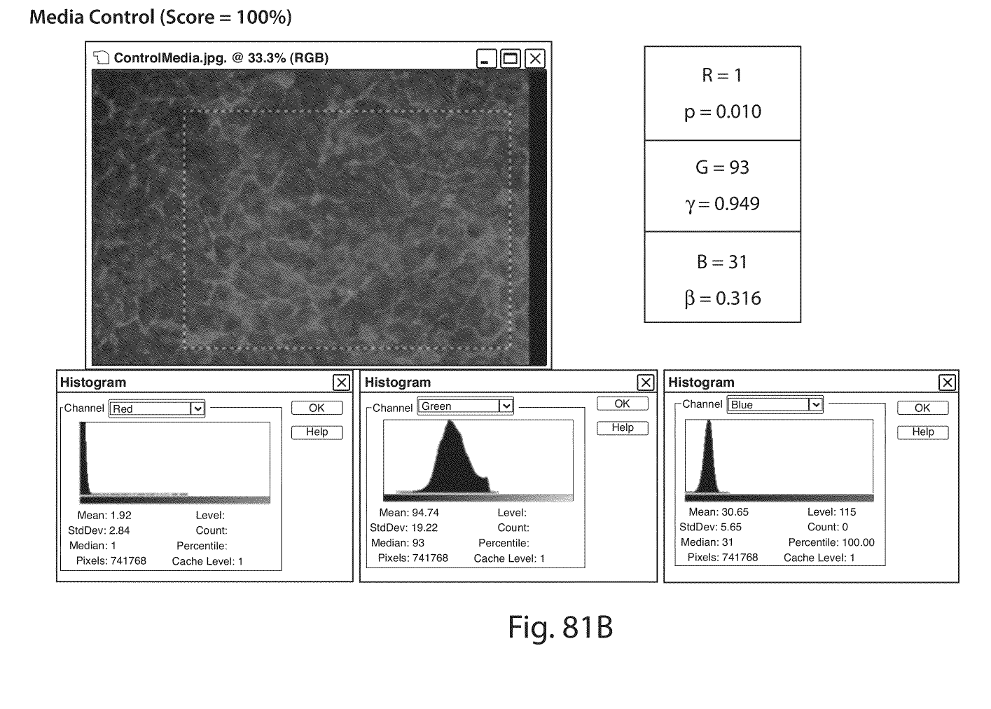

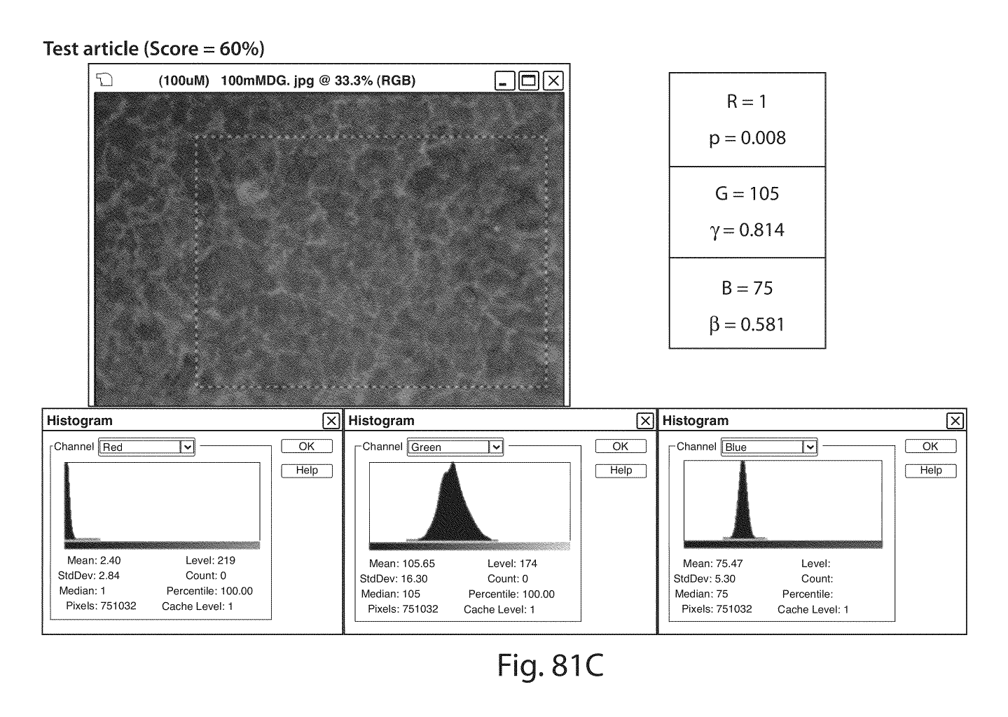

Accelerated renal failure and fibrosis was observed in Alk-3 deleted mice with NTN when compared to the control mice (FIG. 76 E, G, I). It was demonstrated that while THR-123 was successful in controlling renal injury and fibrosis in the control mice (FIG. 76 E, F, I), it has no efficacy in the Alk-3 deleted mice (FIG. 76 E-I). Such therapeutic effects of THR-123 on control mice with NTN was associated with the inhibition of macrophage accumulation in the kidney (FIG. 76 J, K, N) and the EMT program in the tubules (FIG. 76 O, P, S), whereas THR-123 did not inhibit both macrophage accumulation (FIG. 76 L, M, N) and the EMT program (FIG. 76 Q, R, S) in Alk3 deleted mice with NTN. THR-123 inhibit apoptosis in wild type kidney with NTN but had no effect on apoptosis in Alk3 deleted mice (FIG. 79). Finally, THR-123 restored renal function in control mice with NTN, but such reno-protective effect of THR-123 was not realized in Alk3-deleted mice (FIG. 76 T).

### Discussion:

TGF superfamily proteins wield considerable influence on the pathogenesis of renal fibrosis³⁸. For most part, many of the molecules in this family, most importantly TGFβ1 and TGFβ2, have been identified as positive regulators of fibrosis due to their ability to recruit myofibroblasts, facilitate EMT program, influence inflammation and induce epithelial cell apoptosis^{2,5,22,33}. While much focus has been placed on the role of TGFβ1 in fibrosis, many studies in the past decade have also identified that BMP-7 (another molecule in the TGF superfamily) serves to inhibit and reverse fibrosis². BMP-7 action is realized via its anti-inflammatory, anti-apoptosis and EMT suppressive actions^{2,5,22,33}. BMP-7 counter-balances the actions of TGFβ1 via Smad-dependent pathways².

In this regard, BMP-7 can also bind to Alk-6 receptor on osteoblasts and induce bone formation^{7,9,11,12}. In the kidney, tubular epithelial cells predominantly express Alk-3 receptor³⁹. Therefore, an ideal therapeutic molecule would be one that binds to Alk-3 but not to Alk-6 receptor. Additionally, while the levels of BMP-7 decreases in the context of renal injury, the role of Alk-3 in the progression of renal disease is unknown²¹⁻²⁴. Therefore, in this study the role of Alk-3 in renal fibrosis was studied and a strategy was developed to construct new molecules that can bind to Alk-3 but not to Alk-6 and tested these molecules for their mechanism of action and therapeutic efficacy. It was demonstrated that systemic administration of recombinant human BMP-7 reverses renal fibrosis via binding to Alk-3 receptor on the kidney tubular epithelial cells. These results also suggested that expression of BMP-7 and Alk-3 inversely correlate with progression of fibrosis. Collectively, these results suggest that BMP-7 plays a likely reno-protective role in opposition to TGFβ1 action².

Similarly, in this study it was identified that Alk-3 is also a positive regulator of renal health during injury. It responds to renal injury in a protective fashion and its loss augments the progression of renal fibrosis. These results, coupled with the anti-fibrotic activity of BMP-7, provided the necessary rationale to design small peptide mimics of BMP-7 action that bind to Alk-3 receptor.

THR-123, is a novel orally available peptide agonist of Alk-3 receptor and a BMP-7 mimic. THR-123 suppressed progression of kidney disease and reverses established kidney fibrosis.

Importantly, it was demonstrated that a combination of THR-123 and captopril showed dramatic additive therapeutic effect in controlling renal fibrosis associated with diabetic kidney disease. Collectively, these results indicated that THR-123 inhibits inflammation, apoptosis, EMT program and reverses renal fibrosis. This action was mediated by Alk-3 receptor³⁹. It is possible that Alk-2 receptor may also contribute to the action of THR-123, but the genetic mouse model experiments suggest that such Alk-2 mediated activity, if present, is insignificant.

In summary, this study suggests that Alk-3 receptor is a negative regulator of fibrosis when kidney is injured. Such reno-protective property mirrored the action of its ligand, BMP-7, in the kidney. The design AA-123 exploited this synergy and exhibited substantial therapeutic efficacy when administered orally to mice with fibrosis. These pre-clinical studies offer insights into design of possible clinical testing of this agent as an anti-fibrosis drug in the future.

### Method and Materials Used for Example 7:

Reagent

Monoclonal antibody for E-cadherin was purchased from BD Biosciences (Franklin Lakes, N.J.). Polyclonal antibody for FSP1 was provided by Dr. Eric Neilson, Vanderbilt University Medical Center. Mac-1 antibody was purchased from AbD Serotec (Oxford, United Kingdom). Phospho-smad1/5 antibody was purchased from Cell Signaling Technology (Beverly Mass.). Measurements of BUN were performed by QuantiChrom™ Urea Assay Kit (BioAssay System, Hayward, Calif.) or colorimetric kit DIUR-500 QuantiChrom™ Urea Assay Kit (Gentaur, Kampenhout Belgium). Elisa kit for IL-6, IL-8 and ICAM-1 were purchased from R&D system (Minneapolis, Minn.).

Microarray Analysis in NTN Kidney

NTN is induced in C57BL6 mice by pre-immunizing with a subcutaneous injection of normal sheep IgG (200 μg) in complete Freund's adjuvant (day 1) followed by intravenous injection of nephrotoxic serum injection (50 μl, from day 5 to day 7). Mice were sacrificed at 1, 3, 6, 9 weeks after induction of NTN. Total RNA was isolated from the kidney by Trizol/Invitrogen PureLink RNA Mini Kit for RNA extraction. Ten nanogram of total RNA was used for generating complementary cDNA using the TaqMan One-Step RT-PCR Master Mix (Applied Biosystems, Foster City, Calif.). Quantitative PCR was performed to analyze the gene expression profile of BMP7, BMP receptors Alk2, Alk-3, Alk6 and BMPR II, and to BMP-binding proteins chordin, criml, fibrillinl, follistatin, KCP, USAG1, gremlin and noggin are analyzed (Table: Primer Sequences (below)) using ABIprism 7000 (Applied Biosystems).

TABLE 1
Primer sequences

| | Forward primer | Reverse primer |
|---|---|---|
| nail | CTTGTGTCTGCACGACCTGT | AGGAGAATGGCTTCTCACCA |
| TGF | GTGGAATATTGCCGGTGCA | CCATTGAAGCATCTTGGTTCG |
| OL-I | TGTAAACTCCCTCCACCCCA | TCGTCTGTTTCCAGGGTTGG |
| N-EIIIA | ATCCGGGAGCTTTTCCCTG | TGCAAGGCAACCACACTGAC |
| MPR2 | TCCACCTGGGTCATCTCCA | CCCTGTCACTGCCATTGTTG |
| lk2 | TGGCCTGACTGGTTGTCAGA | TTCCGTCAAAGCAGCCACT |
| lk3 | GGACATGCGTGAGGTTGTGT | CGCTGTTCCAGCGGTTAGAC |
| lk6 | GCGGCCTATGCCATTTACAC | AGTCTCGATGGGCGATTGC |
| MP7 | CCTCTGTTCTTGCTGCGCTC | AAGCTGGAGTGCACCTCGTT |
| hordin | GTAGCGAGGTGGTGGCCAT | CAGGACAGTGCGCTGGTTC |
| rim | GGACAGCTACGAAACGCAAGT | CATCTTGCTGGCAGGGTACA |
| ibril- | TCGACGAGTGTCAGAATGGC | TGCCTGCAGTGTTGATGCA |
| lin1 | | |
| olli- | TGCCAGTGACAATGCCACAT | CCAGAAGAGCAGGCAGCTTC |
| statin | | |
| CP | AGTTCCAACCCATGCCTCC | GGCACTTCACAGGCACACAT |
| SAG1 | TTAAACCTGTCCCGGCACA | CTGCCTCCATTCCTGGCTT |
| remlin | TGAAGCAGACCATCCACGAG | GGCCATAACAGAAGCGGTTG |
| oggin | AGCGAGATCAAAGGGCTGG | CTCAGGCGCTGTTTCTTGC |

Conditional Deletion of Alk-3 in Renal Tubule

Alk-3 flox mice are provided by Dr. Yuji Mishina, National Institutes of Health with material transfer agreements. γGT-Cre mice are provided from Dr. Eric Neilson, Vanderbilt University Medical Center. NTN is induced by the method described above.

Ischemic Reperfusion Injury

Eight Weeks Old C5

7B16 mice are used in this study. Mice are anesthetized with the mixture of ketamine and xylazine and the left renal pedicle clamped for 25 minutes. At same day after surgery, THR-123 (p.o. 5 mg/Kg/day) or vehicle treatments are started. At 7 days after surgery, mice are sacrificed.

Unilateral Ureteral Obstruction

Mice are anesthetized with the mixture of ketamine and xylazine. Prepare the ureter from the surrounding tissues and place two ligatures about 5 mm apart in upper two-thirds of the ureter of the left kidney to obtain reliable obstruction. At same day after surgery, mice were initiated treatment with BMP7 (300 μg i.p./Kg/every other day), THR-123 (p.o. 5 mg or 15 mg/Kg/day, i.p. 5 mg/Kg/day) or PBS (i.p.) as control. Mice are sacrificed at day 5 or 7 after operation.

Nephrotoxic Serum Induced Nephritis (NTN)

NTN is induced in CD1 mice by the method described above. Six weeks after NTN induction, THR-123 (p.o. 5 mg/Kg/day) are started until 9 weeks. Mice are sacrificed at week 1, week 3, week 6 and week 9.

For the analysis of glomerulosclerosis, 20 glomeruli per each mouse were randomly picked and each glomerulus was evaluated according to the following scale: no sclerosis 0, 0 to ¼ of a glomerular surface area was sclerosed 1, ¼ to ½ was sclerosed 2, ½ to ¾ was sclerosed 3 and more than ¾ was sclerosed or with crescent 4. A glomerulosclerosis score was calculated as an arithmetic mean of these numbers for each mouse. The glomerulosclerosis scores from all mice were arbitrarily divided into 4 groups; that is no disease, mild, moderate and severe. The percentage of mice with these 4 groups was calculated.

For tubular atrophy score, ten 200× visual fields were randomly selected for each slide and tubular atrophy was assessed according to the following scale: no atrophy 0, 0 to ¼ of a visual field was occupied by atrophied tubules 1, ¼ to ½ 2, ½ to ¾ 3 and more than ¾ 4. A tubular atrophy score was then calculated as an arithmetic mean of these numbers per each mouse. The tubular atrophy scores from all mice were arbitrarily divided into 4 groups; that is no disease, mild, moderate and severe. The percentage of mice with these 4 groups was calculated and shown on the graph.

For the analysis of interstitial fibrosis, ten 200× visual fields were also selected randomly for each Masson trichrome stained kidney section and interstitial fibrosis was evaluated according to the following scale: no fibrosis 0, 0 to ¼ of a visual field was affected by interstitial fibrosis 1, ¼ to ½ 2, ½ to ¾ 3 and more than ¾ 4. A fibrosis index was calculated as an arithmetic mean of these numbers per each mouse. The fibrosis indices from all mice were arbitrarily divided into 4 groups; that is no disease, mild, moderate and severe. The percentage of mice with these 4 groups was calculated.

Type IV Collagen a 3 Chain Knockout Mice (COL4A3^{-/-})

Eight weeks old COL4A3^{-/-} mice are treated either THR-123 (p.o. 5 mg/Kg/day) or vehicle. COL4A3^{-/-} mice are sacrificed at 16 weeks of age.

For morphometric analyses of percent normal glomeruli, 100 glomeruli were counted per slide from random fields of view, and five slides were counted per experimental group. The number of normal glomeruli was expressed as a percentage of the total number of glomeruli counted.

Diabetic Nephropathy

Eight weeks old male CD-1 mice are used all the diabetic experiment. Mice are performed single intraperitoneal (i.p.) injection of streptozotocin (STZ: 200 mg/Kg). Induction of diabetes is defined as blood glucose level >16 mM by 2 weeks after STZ injection. One month after induction of diabetes, mice were separated into three groups (BMP7, vehicle and non-treatment) and BMP7 (i.p. 300 μg/Kg/every other day) or vehicle injection was initiated. Five months after induction of diabetes, THR-123 (p.o. 5 mg/Kg/day) administration is initiated in diabetic mice. Mice are sacrificed at 5 (before the treatment) or 6 months after induction of diabetes.

For the captopril (CPR) and THR-123 combination therapy trial, diabetic mice are separated into three groups at 7 months after induction of diabetes (vehicle, CPR and CPR-THR-123 combination). CPR (p.o. 50 mg/Kg/day) or combination of CPR and THR-123 (p.o. 5 mg/Kg/day) treatments were initiated. Mice are sacrificed at 7 (before the treatment) or 8 months after induction of diabetes.

For glomerular damage, we evaluated mesangial matrix expansion and enlargement of the glomeruli. A point counting method was used to quantify mesangial matrix deposition. 20 PAS-stained glomeruli from each mouse were analyzed on a digital microscope screen grid containing 667 (29×23) points. The number of grid points that hit pink or red mesangial matrix deposition were divided by the total number of points in the glomerulus to obtain the percentage of mesangial matrix deposition in a given glomerulus.

Morphometric Analysis

Kidney sections were stained with hematoxylin and eosin, Masson's trichrome, and periodic acid-Schiff. The extent of renal injury was estimated by morphometric assessment of the tubulointerstitial injury and glomerular damage. The relative interstitial volume was evaluated by morphometric analysis using a 10-mm² graticule fitted into the microscope. Five to ten randomly selected cortical areas were evaluated for each animal. Three hundred to five hundred tubules were evaluated for their widened lumen and thickened basement membranes to estimate percentage of atrophic tubules. This method was used for UUO, COL4A3KO and diabetic study.

Detection of LacZ

Kidney samples (1 mm²) from 6 week old R26Rstop LacZ flox mice²⁷ with or without -Cre were fixed at 4° C. for 4 h in 4% paraformaldehyde. Samples were washed 3 times with PBS pH 7.3 and then stained overnight at 37° C. with LacZ staining buffer (1 mg/ml X-gal, 35 mM potassium ferrocyanide, 35 mM potassium ferricyanide, 2 mM MgCl₂, 0.02% NP-40, 0.01% sodium deoxycholate in PBS). After washing with PBS pH 7.3, samples were embedded into paraffin. Sections (10 μm) were then deparaffinized and counterstained with eosin.

In Vitro EMT

EMT was induced in NP1 cells or MCT cells by incubation with 3 ng/ml recombinant human TGF-μ1 for 48 h. When EMT occurred, the medium was removed and replaced with THR-123 (10 μM) or recombinant human BMP7 (100 ng/ml) in DMEM. After 48 h, the cells were characterized by immunocytochemistry using primary monoclonal antibodies to E-cadherin (2.5 g/ml) and rhodamine-conjugated secondary antibodies (Jackson Immunoresearch, West Grove, Pa.) as previously described. The staining was visualized by fluorescence microscopy and documented representative pictures using Spot advanced software (Carl Zeiss, Oberkochen, Germany). Also protein and total RNA were harvested at the end of experiment. For the morphometric analysis for EMT, length/width of cells in bright field pictures are measured by image J software. The ratio of length/width was calculated.

Inflammatory Cytokine Production

Human proximal tubular epithelial cells-derived HK-2 cells were culture on 24-well plate (30,000 cells/well). Cells are exposed to K-SFM medium alone or TNF-β (5 ng/ml). Twenty hours after TNF-α incubation, cells are washed twice by pre-warmed culture media and subsequently cells are incubated with various concentration of THR-123 or BMP7 for 60 hours. At the end of incubation, culture medias are harvested and ELISA analysis for IL-6, IL-8 and ICAM-1 are performed.

Apoptosis

HK-2 cells are passaged on 24-well plates (25,000-30,000 cell/well). Once cells attached on the well, cells are exposed either K-SFM media alone or K-SFM medium containing THR-123. BMP7 serves as a positive control of experiment. Two hours after incubation, cells are exposed to cisplatin for 60 hours. Apoptosis is determined by staining of AnnexinV-FITC, followed by fluorescence microscopy. Final concentration: THR-123 250 μM, BMP7 1 μg/ml, cisplatin 10 μM.

Statistical Analysis

The data are expressed as means±s.e.m. Analysis of variance (ANOVA) followed by Bonferroni/Dunn's test for multiple comparisons of mouse samples were used to determine significant. Statistical significance was defined as P<0.05. Graph-pad Prism software was used for statistical analysis.

### REFERENCE AS CITED IN EXAMPLE 7

The following references are cited in Example 7, the contents each of which are incorporated herein by reference.
1 Zeisberg, E. M. et al. Endothelial-to-mesenchymal transition contributes to cardiac fibrosis. Nat Med 13, 952-961 (2007).
2 Zeisberg, M. et al. BMP-7 counteracts TGF-beta1-induced epithelial-to-mesenchymal transition and reverses chronic renal injury. Nat Med 9, 964-968 (2003).
3 Zeisberg, M. & Kalluri, R. The role of epithelial-to-mesenchymal transition in renal fibrosis. J Mol Med 82, 175-181 (2004).
4 Miyazono, K., Maeda, S. & Imamura, T. BMP receptor signaling: transcriptional targets, regulation of signals, and signaling cross-talk. Cytokine Growth Factor Rev 16, 251-263 (2005).
5 Simic, P. & Vukicevic, S. Bone morphogenetic proteins in development and homeostasis of kidney. Cytokine Growth Factor Rev 16, 299-308 (2005).
6 Simic, P. & Vukicevic, S. Bone morphogenetic proteins: from developmental signals to tissue regeneration. Conference on bone morphogenetic proteins. EMBO Rep 8, 327-331 (2007).
7 Ide, H. et al. Growth regulation of human prostate cancer cells by bone morphogenetic protein-2. Cancer Res 57, 5022-5027 (1997).
8 Chen, D. et al. Differential roles for bone morphogenetic protein (BMP) receptor type IB and IA in differentiation and specification of mesenchymal precursor cells to osteoblast and adipocyte lineages. J Cell Biol 142, 295-305 (1998).
9 Haaijman, A. et al. Correlation between ALK-6 (BMPR-IB) distribution and responsiveness to osteogenic protein-1 (BMP-7) in embryonic mouse bone rudiments. Growth Factors 17, 177-192 (2000).
10 Zhao, M. et al. Bone morphogenetic protein receptor signaling is necessary for normal murine postnatal bone formation. J Cell Biol 157, 1049-1060, doi:10.1083/jcb.200109012 (2002).
11 Archdeacon, P. & Detwiler, R. K. Bone morphogenetic protein 7 (BMP7): a critical role in kidney development and a putative modulator of kidney injury. Adv Chronic Kidney Dis 15, 314-320 (2008).
12 Hojo, H., Ohba, S., Yano, F. & Chung, U. I. Coordination of chondrogenesis and osteogenesis by hypertrophic chondrocytes in endochondral bone development. J Bone Miner Metab 28, 489-502, doi:10.1007/s00774-010-0199-7 (2010).
13 Mackensen-Haen, S., Bader, R., Grund, K. E. & Bohle, A. Correlations between renal cortical interstitial fibrosis, atrophy of the proximal tubules and impairment of the glomerular filtration rate. Clin Nephrol 15, 167-171 (1981).
14 Nath, K. A. Tubulointerstitial changes as a major determinant in the progression of renal damage. Am J Kidney Dis 20, 1-17 (1992).
15 Risdon, R. A., Sloper, J. C. & de Wardener, H. E. Relationship between renal function and histological changes found in renal biopsy specimens from patients with persistent glomerular nephritis. Lancet 2, 363 (1968).
16 Schainuck, L. I., Striker, G. E., Cutler, R. E. & Benditt, E. P. Structural-functional correlations in renal disease. II. The correlations. Hum Pathol 1, 631-641 (1970).
17 Striker, G. E., Schainuck, L. I., Cutler, R. E. & Benditt, E. P. Structural-functional correlations in renal disease. I. A method for assaying and classifying histopathologic changes in renal disease. Hum Pathol 1, 615-630 (1970).
18 Zeisberg, M. et al. Bone morphogenic protein-7 inhibits progression of chronic renal fibrosis associated with two genetic mouse models. Am J Physiol Renal Physiol 285, F1060-1067 (2003).
19 Zeisberg, M., Shah, A. A. & Kalluri, R. Bone morphogenic protein-7 induces mesenchymal to epithelial transition in adult renal fibroblasts and facilitates regeneration of injured kidney. J Biol Chem 280, 8094-8100 (2005).
20 Sugimoto, H., Grahovac, G., Zeisberg, M. & Kalluri, R. Renal fibrosis and glomerulosclerosis in a new mouse model of diabetic nephropathy and its regression by bone morphogenic protein-7 and advanced glycation end product inhibitors. Diabetes 56, 1825-1833 (2007).
21 Simon, M. et al. Expression of bone morphogenetic protein-7 mRNA in normal and ischemic adult rat kidney. Am J Physiol 276, F382-389 (1999).
22 Vukicevic, S. et al. Osteogenic protein-1 (bone morphogenetic protein-7) reduces severity of injury after ischemic acute renal failure in rat. J Clin Invest 102, 202-214 (1998).
23 Wang, S. N., Lapage, J. & Hirschberg, R. Loss of tubular bone morphogenetic protein-7 in diabetic nephropathy. J Am Soc Nephrol 12, 2392-2399 (2001).
24 Tuglular, S. et al. Cyclosporine-A induced nephrotoxicity is associated with decreased renal bone morphogenetic protein-7 expression in rats. Transplant Proc 36, 131-133, doi:10.1016/j.transproceed.2003.11.18 (2004).
25 Iwano, M. et al. Evidence that fibroblasts derive from epithelium during tissue fibrosis. J Clin Invest 110, 341-350 (2002).
26 Mishina, Y., Hanks, M. C., Miura, S., Tallquist, M. D. & Behringer, R. R. Generation of Bmpr/Alk3 conditional knockout mice. Genesis 32, 69-72 (2002).
27 Srinivas, S. et al. Cre reporter strains produced by targeted insertion of EYFP and ECFP into the ROSA26 locus. BMC Dev Biol 1, 4 (2001).
28 Rodriguez-Iturbe, B. & Garcia Garcia, G. The role of tubulointerstitial inflammation in the progression of chronic renal failure. Nephron Clin Pract 116, c81-88 (2010).
29 Daopin, S., Piez, K. A., Ogawa, Y. & Davies, D. R. Crystal structure of transforming growth factor-beta 2: an unusual fold for the superfamily. Science 257, 369-373 (1992).
30 Schlunegger, M. P. & Grutter, M. G. An unusual feature revealed by the crystal structure at 2.2 A resolution of human transforming growth factor-beta 2. Nature 358, 430-434 (1992).
31 Griffith, D. L., Keck, P. C., Sampath, T. K., Rueger, D. C. & Carlson, W. D. Three-dimensional structure of recombinant human osteogenic protein 1: structural paradigm for the transforming growth factor beta superfamily. Proc Natl Acad Sci USA 93, 878-883 (1996).
32 Keck, P. C. Computer Method And Apparatus For Classifying Objects. WO/2002/037313. (2002).
33 Gould, S. E., Day, M., Jones, S. S. & Dorai, H. BMP-7 regulates chemokine, cytokine, and hemodynamic gene expression in proximal tubule cells. Kidney Int 61, 51-60 (2002).
34 Lloyd, C. M. et al. RANTES and monocyte chemoattractant protein-1 (MCP-1) play an important role in the inflammatory phase of crescentic nephritis, but only MCP-1 is involved in crescent formation and interstitial fibrosis. J Exp Med 185, 1371-1380 (1997).
35 Kashtan, C. E. Alport syndrome. An inherited disorder of renal, ocular, and cochlear basement membranes. Medicine (Baltimore) 78, 338-360 (1999).
36 Lewis, E. J., Hunsicker, L. G., Bain, R. P. & Rohde, R. D. The effect of angiotensin-converting-enzyme inhibition on diabetic nephropathy. The Collaborative Study Group. N Engl J Med 329, 1456-1462 (1993).
37 Parving, H. H., Hommel, E., Damkjaer Nielsen, M. & Giese, J. Effect of captopril on blood pressure and kidney function in normotensive insulin dependent diabetics with nephropathy. Bmj 299, 533-536 (1989).
38 Border, W. A. & Noble, N. A. Transforming growth factor beta in tissue fibrosis. N Engl J Med 331, 1286-1292, doi:10.1056/NEJM199411103311907 (1994).
39 Wetzel, P. et al. Bone morphogenetic protein-7 expression and activity in the human adult normal kidney is predominantly localized to the distal nephron. Kidney Int 70, 717-723, doi:10.1038/sj.ki.5001653 (2006).

### ADDITIONAL REFERENCES CITED HEREIN

The following references are cited throughout the specification, the contents each of which are incorporated herein by reference.
1. LeRoy, E. C., Trojanowska, M. I., and Smith, E. A. (1990) Cytokines and human fibrosis. Eur. Cytokine Netw. 1, 215-219
2. Bradham, D. M., Igarashi, A., Potter, R. L., and Grotendorst, G. R. (1991) Connective tissue growth factor: a cysteine-rich mitogen secreted by human vascular endothelial cells is related to the SRC-induced immediate early gene product CEF-10. J. Cell Biol. 114, 1285-1294
3. Leask, A., Denton, C. P., and Abraham, D. J. (2004) Insights into the molecular mechanism of sustained fibrosis: The role of connective tissue growth factor in scleroderma. J. Invest. Dermatol. 122, 1-6
4. Grotendorst, G. R. (1997) Connective tissue growth factor: a mediator of TGF-beta action on fibroblasts. Cytokine GrowthFactor Rev. 8, 171-179
5. Oyama, F., Murata, Y., Suganuma, N., Kimura, T., Titani, K., and Sekiguchi, K. (1989) Patterns of alternative splicing of fibronectin pre-mRNA in human adult and fetal tissues. Biochemistry 28, 1428-1434
6. Serini, G., Bochaton-Piallat, M. L., Ropraz, P., Geinoz, A., Borsi, L., Zardi, L., and Gabbiani, G. (1998) The fibronectin domain ED-A is crucial for myofibroblastic phenotype induction by transforming growth factor-beta1. J. Cell Biol. 142, 873-881
7. Sime P J, O'Reilly K M A. Fibrosis of the lung and other tissues: new concepts in pathogenesis and treatment. Clin Immunol 99: 308-319,2001.
8. Zhao J, Shi W, Wang Y L, Chen H, Bringas P Jr, Datto M B, Frederick J P, Wang X F, Warburton D. Smad3 deficiency attenuates bleomycin-induced pulmonary fibrosis in mice. Am J Physiol Lung Cell Mol Physiol 282: L585-L593, 2002.
9. Miettinen P J, Ebner R, Lopez A R, Derynck R. TGF-beta induced transdifferentiation of mammary epithelial cells to mesenchymal cells: involvement of type I receptors. J Cell Biol 127: 2021-2036, 1994.
10. Fan J M, Ng Y Y, Hill P A, Nikolic-Paterson D J, Mu W, Atkins R C, Lan H Y. Transforming growth factor-_{—} regulates tubular epithelialmyofibroblast transdifferentiation in vitro. Kidney Int 56: 1455-1467, 1999
11. Hales A M, Schulz M W, Chamberlain C G, McAvoy J W. TGF-beta 1 induces lens cells to accumulate alpha-smooth muscle actin, a marker for subcapsular cataracts. Curr Eye Res 13: 885-890, 1994.
12. Kasai H, Allen J T, Mason R M, Kamimura T, Zhang Z. TGF-_{—}1 induces human alveolar epithelial to mesenchymal cell transition (EMT). Respir Res 6: 56, 2005.
13. Saika S, Kono-Saika S, Ohnishi Y, Sato M, Muragaki Y, Ooshima A, Flanders K C, Yoo J, Anzano M, Liu C Y, Kao W W, Roberts A B. Smad3 signaling is required for epithelial-mesenchymal transition of lens epithelium after injury. Am J Pathol 164: 651-663, 2004.
14. Willis B C, Liebler J M, Luby-Phelps K, Nicholson A G, Crandall E D, du Bois R M, Borok Z. Induction of epithelial-mesenchymal transition in alveolar epithelial cells by transforming growth factor-beta1: potential role in idiopathic pulmonary fibrosis. Am J Pathol 166: 1321-1332, 2005
15. Saika S, Kono-Saika S, Ohnishi Y, Sato M, Muragaki Y, Ooshima A, Flanders K C, Yoo J, Anzano M, Liu C Y, Kao W W, Roberts A B. Smad3 signaling is required for epithelial-mesenchymal transition of lens epithelium after injury. Am J Pathol 164: 651-663, 2004.
16. Sato M, Muragaki Y, Saika S, Roberts A B, Ooshima A. Targeted disruption of TGF-_{—}1/Smad3 signaling protects against renal tubulointerstitial fibrosis induced by unilateral ureteral obstruction. J Clin Invest 112: 1486-1494, 2003.
17. Phanish M K, Wahab N A, Colville-Nash P, Hendry B M, Dockrell M E. The differential role of Smad2 and Smad3 in the regulation of pro-fibrotic TGF β1 responses in human proximal-tubule epithelial cells. Biochem J 393: 601-607, 2006.
18. Valcourt U, Kowanetz M, Niimi H, Heldin C H, Moustakas A. TGF-beta and the Smad signaling pathway support transcriptomic reprogramming during epithelial-mesenchymal cell transition. Mol Biol Cell 16: 1987-2002, 2005.
19. Zavadil J, Bottinger E P. TGF-β and epithelial-to-mesenchymal transitions. Oncogene 24: 5764-5774, 2005.
20 Zeisberg M, Shah A A, Kalluri R. Bone morphogenic protein-7 induces mesenchymal to epithelial transition in adult renal fibroblasts and facilitates regeneration of injured kidney. J Biol Chem 280: 8094-8100, 2005.
21 Saika S, Ikeda K, Yamanaka 0, Flanders K C, Ohnishi Y, Nakajima Y, Muragaki Y, Ooshima A. Adenoviral gene transfer of BMP-7, Id2, or Id3 suppresses injury-induced epithelial-to-mesenchymal transition of lens epithelium in mice. Am J Physiol Cell Physiol 290: C282-C289, 2006.
22. Zeisberg M, Hanai Ji Sugimoto H, Mammoto T, Charytan D, Strutz F, Kalluri R. BMP-7 counteracts TGF-β1-induced epithelial-to-mesenchymal transition and reverses chronic renal injury. Nat Med 9: 964-968, 2003.
23. Yang J, Dai C, Liu Y. A novel mechanism by which hepatocyte growth factor blocks tubular epithelial to mesenchymal transition. J Am Soc Nephrol 16: 68-78, 2005.
24. Hay E D. An overview of epithelio-mesenchymal transformation. Acta Anat 154: 8-20, 1995.
25. Vanderburg C R, Hay E D. E-cadherin transforms embryonic corneal fibroblasts to stratified epithelium with desmosomes. Acta Anat 157: 87-104, 1996.
26. Hills C E, Siamantouras E, Smith S W, Cockwell P, Liu K K, Squires P E. TGFβ modulates cell-to-cell communication in early epithelial-to-mesenchymal transition. Diabetologia. 2012 March; 55(3):812-24.
27. Gharaee-Kermani M, Hu B, Phan S H, Gyetko M R. Recent advances in molecular targets and treatment of idiopathic pulmonary fibrosis: focus on TGF beta signaling and the myofibroblast. Curr Med Chem. 2009; 16(11):1400-17.
28. Willis B C, duBois R M, Borok Z. Epithelial origin of myofibroblasts during fibrosis in the lung. Proc Am Thorac Soc. 2006 June; 3(4):377-82
29. Kevin K. Kim, Ying Wei, Charles Szekeres, Matthias C. Kugler, Paul J. Wolters, Marla L. Hill, James A. Frank, Alexis N. Brumwell, Sarah E. Wheeler, Jordan A. Kreidberg, and Harold A. Chapman. Epithelial cell α3β1 integrin links β-catenin and Smad signaling to promote myofibroblast formation and pulmonary fibrosis.) Clin Invest. 2009 Jan. 5; 119(1): 213-224.
30. Zeisberg M, Hanai J, Sugimoto H, Mammoto T, Charytan D, Strutz F, Kalluri R. BMP-7 counteracts TGF-beta1-induced epithelial-to-mesenchymal transition and reverses chronic renal injury. Nat Med. 2003 July; 9(7):964-8.
31. Myllarniemi M, Lindholm P, Ryynanen M J, Kliment C R, Salmenkivi K, Keski-Oja J, Kinnula V L, Oury T D, Koli K. Gremlin-mediated decrease in bone morphogenetic protein signaling promotes pulmonary fibrosis. Am J Respir Crit Care Med. 2008 Feb. 1; 177(3):321-9.
32. Xu Y, Wan J, Jiang D, Wu X. BMP-7 counteracts TGF-beta1-induced epithelial-to-mesenchymal transition in human renal proximal tubular epithelial cells. J Nephrol. 2009 May-June; 22(3):403-10.
33. Zeisberg M, Kalluri R. Reversal of experimental renal fibrosis by BMP7 provides insights into novel therapeutic strategies for chronic kidney disease. Pediatr Nephrol. 2008 September; 23(9):1395-8.
34. Sugimoto H, Grahovac G, Zeisberg M, Kalluri R. Renal fibrosis and glomerulosclerosis in a new mouse model of diabetic nephropathy and its regression by bone morphogenic protein-7 and advanced glycation end product inhibitors. Diabetes. 2007 July; 56(7):1825-33.
35. Zeisberg M, Bottiglio C, Kumar N, Maeshima Y, Strutz F, Müller G A, Kalluri R. Bone morphogenic protein-7 inhibits progression of chronic renal fibrosis associated with two genetic mouse models. Am J Physiol Renal Physiol. 2003 December; 285(6):F1060-7
36. Kalluri R, Zeisberg M. Exploring the connection between chronic renal fibrosis and bone morphogenic protein-7. Histol Histopathol. 2003 January; 18(1):217-24
37. Izumi N, Mizuguchi S Inagaki, Y, Saika S, Kawada N, Nakajima Y, Kiyotoshi, Inoue K, Suehiro S, Friedman, S L and Ikeda K. BMP-7 opposes TGF-_{—}1-mediated collagen induction in mouse pulmonary myofibroblasts through Id2. Am J Physiol Lung Cell Mol Physiol 290: L120-L126, 2006.
38. Cao H, Shu X, Chen L B, Zhang K, Xu Q H, Li G. The relationship of expression of BMP-7 in the liver and hepatic inflammation and fibrosis in patients with chronic HBV infection. Zhonghua Shi Yan He Lin Chuang Bing Du Xue Za Zhi. 2010 April; 24(2):101-3.
39. Zeisberg E M, Tarnayski 0, Zeisberg M, Dorfman A L, McMullen J R, Gustafsson E, Chandraker A, Yuan X, Pu W T, Roberts A B, Neilson E G, Sayegh M H, Izumo S, Kalluri R. Endothelial-to-mesenchymal transition contributes to cardiac fibrosis. Nat Med. 2007 August; 13(8):952-61
40. Davies M R, Lund R J, Hruska K A. BMP-7 is an efficacious treatment of vascular calcification in a murine model of atherosclerosis and chronic renal failure. J Am Soc Nephrol. 2003 June; 14(6):1559-67
41. Sugimoto H, LeBleu V S, Bosukonda D, Keck P, Taduri G, Bechtel W, Okada H, Carlson W Jr, Bey P, Rusckowski M, Tampe B, Tampe D, Kanasaki K, Zeisberg M, Kalluri R. Activin-like kinase 3 is important for kidney regeneration and reversal of fibrosis. Nat Med. 2012 Feb. 5; 18(3):396-404.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a subject having a disease or disorder characterized by fibrosis, comprising administering to the subject an effective amount of a peptide comprising SEQ ID NO:4, thereby treating the subject.

2.-3. (canceled)

4. The method of claim 1, wherein the peptide is formulated with a pharmaceutically acceptable carrier.

5. The method of claim 1, wherein the peptide is administered to the subject orally.

6. The method of claim 1, wherein the peptide is administered to the subject topically, enterally, or parenterally.

7. The method of claim 1, wherein the disease or disorder is diabetic nephropathy, liver cirrhosis, idiopathic pulmonary fibrosis, rheumatoid arthritis, atherosclerosis, cardiac fibrosis, systemic sclerosis, nepthritis, and scleroderma.

8. The method of claim 1, wherein the disease or disorder is chronic kidney disease (CKD).

9. The method of claim 1, wherein a dosage of 0.0001 to 10,000 mg/kg body weight is administered to the subject per day.

10. The method of claim 11, wherein the administered dosage is from 1 to 100 mg/kg body weight per day.

11. A peptide for treating a disease or disorder characterized by fibrosis comprising the amino acid sequence set forth as SEQ ID NO:4.

12. (canceled)

13. The peptide of claim 11, consisting of the amino acid sequence set forth as SEQ ID NO:4.

14. A pharmaceutical composition comprising the peptide of claim 11 and a pharmaceutically acceptable carrier.

15. A kit comprising the peptide claim 11 and instructions for use.

16. A kit comprising the pharmaceutical composition of claim 14 and instructions for use.

17. A pharmaceutical composition comprising the peptide of claim 13 and a pharmaceutically acceptable carrier.

18. A kit comprising the peptide of claim 13 and instructions for use.

19. A kit comprising the pharmaceutical composition of claim 17 and instructions for use.


SEQUENCE LISTING (111 sequences)

SEQ ID NO: 1 - protein, 16 aa, Artificial Sequence
  Description of Artificial Sequence Synthetic peptide
CYFDDSSNVLCKKYRS

SEQ ID NO: 2 - protein, 16 aa, Artificial Sequence
  Description of Artificial Sequence Synthetic peptide
DYFDDSSNVLXKKYRS

SEQ ID NO: 3 - protein, 16 aa, Artificial Sequence
  Description of Artificial Sequence Synthetic peptide
XYFDDSSNVLDKKYRS

SEQ ID NO: 4 - protein, 16 aa, Artificial Sequence
  Description of Artificial Sequence Synthetic peptide
CYYDNSSSVLCKRYRS

SEQ ID NO: 5 - protein, 16 aa, Artificial Sequence
  Description of Artificial Sequence Synthetic peptide
CYYDNSSSVLCKRYRS

SEQ ID NO: 6 - protein, 16 aa, Artificial Sequence
  Description of Artificial Sequence Synthetic peptide
CYYDNSSSVLCKRYRS

SEQ ID NO: 7 - protein, 16 aa, Artificial Sequence
  Description of Artificial Sequence Synthetic peptide
CYYDNSSSVLCKRYRS

SEQ ID NO: 8 - protein, 16 aa, Artificial Sequence
  Description of Artificial Sequence Synthetic peptide
CYYDNSSSVLCKKYRS

SEQ ID NO: 9 - protein, 16 aa, Artificial Sequence
  Description of Artificial Sequence Synthetic peptide
XYYDNSSSVLDKRYRS

SEQ ID NO: 10 - protein, 16 aa, Artificial Sequence
  Description of Artificial Sequence Synthetic peptide
CYFNDSSQVLCKRYRS

SEQ ID NO: 11 - protein, 16 aa, Artificial Sequence
  Description of Artificial Sequence Synthetic peptide
CYYDDSSSVLCKKYRS

SEQ ID NO: 12 - protein, 16 aa, Artificial Sequence
  Description of Artificial Sequence Synthetic peptide
CYFDDSSQVLCKKYRS

SEQ ID NO: 13 - protein, 16 aa, Artificial Sequence
  Description of Artificial Sequence Synthetic peptide
CYFDDSSSVLCKKYRS

SEQ ID NO: 14 - protein, 16 aa, Artificial Sequence
  Description of Artificial Sequence Synthetic peptide
CYFNDSSNVLCKKYRS

SEQ ID NO: 15 - protein, 16 aa, Artificial Sequence
  Description of Artificial Sequence Synthetic peptide
CYYDDSSNVLCKKYRS

SEQ ID NO: 16 - protein, 16 aa, Artificial Sequence
  Description of Artificial Sequence Synthetic peptide
CYFDDSSQVLCKRYRS

SEQ ID NO: 17 - protein, 16 aa, Artificial Sequence
  Description of Artificial Sequence Synthetic peptide
CYYDDSSQVLCKRYRS

SEQ ID NO: 18 - protein, 16 aa, Artificial Sequence
  Description of Artificial Sequence Synthetic peptide
CYFDNSSQVLCKRYRS

SEQ ID NO: 19 - protein, 16 aa, Artificial Sequence
  Description of Artificial Sequence Synthetic peptide
CYFDNSSSVLCKRYRS

SEQ ID NO: 20 - protein, 16 aa, Artificial Sequence
  Description of Artificial Sequence Synthetic peptide
CYFDNSSSVLCKKYRS

SEQ ID NO: 21 - protein, 16 aa, Artificial Sequence
  Description of Artificial Sequence Synthetic peptide
CYFDDSSNVLCKRYRS

SEQ ID NO: 22 - protein, 16 aa, Artificial Sequence
  Description of Artificial Sequence Synthetic peptide
CYFDDSSSVLCKRYRS

SEQ ID NO: 23 - protein, 16 aa, Artificial Sequence
  Description of Artificial Sequence Synthetic peptide
CYFDNSSNVLCKKYRS

SEQ ID NO: 24 - protein, 16 aa, Artificial Sequence
  Description of Artificial Sequence Synthetic peptide
CYFDNSSNVLCKRYRS

SEQ ID NO: 25 - protein, 16 aa, Artificial Sequence
  Description of Artificial Sequence Synthetic peptide
CYFDNSSQVLCKKYRS

SEQ ID NO: 26 - protein, 16 aa, Artificial Sequence
  Description of Artificial Sequence Synthetic peptide
CYFNDSSNVLCKRYRS

SEQ ID NO: 27 - protein, 16 aa, Artificial Sequence
  Description of Artificial Sequence Synthetic peptide
CYFNDSSQVLCKKYRS

SEQ ID NO: 28 - protein, 16 aa, Artificial Sequence
  Description of Artificial Sequence Synthetic peptide
CYFNDSSSVLCKKYRS

SEQ ID NO: 29 - protein, 16 aa, Artificial Sequence
  Description of Artificial Sequence Synthetic peptide
CYFNDSSSVLCKRYRS

SEQ ID NO: 30 - protein, 16 aa, Artificial Sequence
  Description of Artificial Sequence Synthetic peptide
CYFNNSSNVLCKKYRS

SEQ ID NO: 31 - protein, 16 aa, Artificial Sequence
  Description of Artificial Sequence Synthetic peptide
CYFNNSSNVLCKRYRS

SEQ ID NO: 32 - protein, 16 aa, Artificial Sequence
  Description of Artificial Sequence Synthetic peptide
CYFNNSSQVLCKKYRS

SEQ ID NO: 33 - protein, 16 aa, Artificial Sequence
  Description of Artificial Sequence Synthetic peptide
CYFNNSSQVLCKRYRS

SEQ ID NO: 34 - protein, 16 aa, Artificial Sequence
  Description of Artificial Sequence Synthetic peptide
CYFNNSSSVLCKKYRS

SEQ ID NO: 35 - protein, 16 aa, Artificial Sequence
  Description of Artificial Sequence Synthetic peptide
CYFNNSSSVLCKRYRS

SEQ ID NO: 36 - protein, 16 aa, Artificial Sequence
  Description of Artificial Sequence Synthetic peptide
CYYDDSSNVLCKRYRS

SEQ ID NO: 37 - protein, 16 aa, Artificial Sequence
  Description of Artificial Sequence Synthetic peptide
CYYDDSSQVLCKKYRS

SEQ ID NO: 38 - protein, 16 aa, Artificial Sequence
  Description of Artificial Sequence Synthetic peptide
CYYDDSSSVLCKRYRS

SEQ ID NO: 39 - protein, 16 aa, Artificial Sequence
  Description of Artificial Sequence Synthetic peptide
CYYDNSSNVLCKKYRS

SEQ ID NO: 40 - protein, 16 aa, Artificial Sequence
  Description of Artificial Sequence Synthetic peptide
CYYDNSSNVLCKRYRS

SEQ ID NO: 41 - protein, 16 aa, Artificial Sequence
  Description of Artificial Sequence Synthetic peptide
CYYDNSSQVLCKKYRS

SEQ ID NO: 42 - protein, 16 aa, Artificial Sequence
  Description of Artificial Sequence Synthetic peptide
CYYDNSSQVLCKRYRS

SEQ ID NO: 43 - protein, 16 aa, Artificial Sequence
  Description of Artificial Sequence Synthetic peptide
CYYNDSSNVLCKKYRS

SEQ ID NO: 44 - protein, 16 aa, Artificial Sequence
  Description of Artificial Sequence Synthetic peptide
CYYNDSSNVLCKRYRS

SEQ ID NO: 45 - protein, 16 aa, Artificial Sequence
  Description of Artificial Sequence Synthetic peptide
CYYNDSSQVLCKKYRS

SEQ ID NO: 46 - protein, 16 aa, Artificial Sequence
  Description of Artificial Sequence Synthetic peptide
CYYNDSSQVLCKRYRS

SEQ ID NO: 47 - protein, 16 aa, Artificial Sequence
  Description of Artificial Sequence Synthetic peptide
CYYNDSSSVLCKKYRS

SEQ ID NO: 48 - protein, 16 aa, Artificial Sequence
  Description of Artificial Sequence Synthetic peptide
CYYNDSSSVLCKRYRS

SEQ ID NO: 49 - protein, 16 aa, Artificial Sequence
  Description of Artificial Sequence Synthetic peptide
CYYNNSSNVLCKKYRS

SEQ ID NO: 50 - protein, 16 aa, Artificial Sequence
  Description of Artificial Sequence Synthetic peptide
CYYNNSSNVLCKRYRS

SEQ ID NO: 51 - protein, 16 aa, Artificial Sequence
  Description of Artificial Sequence Synthetic peptide
CYYNNSSQVLCKKYRS

SEQ ID NO: 52 - protein, 16 aa, Artificial Sequence
  Description of Artificial Sequence Synthetic peptide
CYYNNSSQVLCKRYRS

SEQ ID NO: 53 - protein, 16 aa, Artificial Sequence
  Description of Artificial Sequence Synthetic peptide
CYYNNSSSVLCKKYRS

SEQ ID NO: 54 - protein, 16 aa, Artificial Sequence
  Description of Artificial Sequence Synthetic peptide
CYYNNSSSVLCKRYRS

SEQ ID NO: 55 - protein, 16 aa, Artificial Sequence
  Description of Artificial Sequence Synthetic peptide
CYXXXXSXVXCKXYRS

SEQ ID NO: 56 - protein, 16 aa, Artificial Sequence
  Description of Artificial Sequence Synthetic peptide
CYYXXXSXVXCKXYRS

SEQ ID NO: 57 - protein, 16 aa, Artificial Sequence
  Description of Artificial Sequence Synthetic peptide
CYFXXXSXVXCKXYRS

SEQ ID NO: 58 - protein, 16 aa, Artificial Sequence
  Description of Artificial Sequence Synthetic peptide
CYXNXXSXVXCKXYRS

SEQ ID NO: 59 - protein, 16 aa, Artificial Sequence
  Description of Artificial Sequence Synthetic peptide
CYXDXXSXVXCKXYRS

SEQ ID NO: 60 - protein, 16 aa, Artificial Sequence
  Description of Artificial Sequence Synthetic peptide
CYXXNXSXVXCKXYRS

SEQ ID NO: 61 - protein, 16 aa, Artificial Sequence
  Description of Artificial Sequence Synthetic peptide
CYXXDXSXVXCKXYRS

SEQ ID NO: 62 - protein, 16 aa, Artificial Sequence
  Description of Artificial Sequence Synthetic peptide
CYXXXSSXVXCKXYRS

SEQ ID NO: 63 - protein, 16 aa, Artificial Sequence
  Description of Artificial Sequence Synthetic peptide
CYXXXNSXVXCKXYRS

SEQ ID NO: 64 - protein, 16 aa, Artificial Sequence
  Description of Artificial Sequence Synthetic peptide
CYXXXXSSVXCKXYRS

SEQ ID NO: 65 - protein, 16 aa, Artificial Sequence
  Description of Artificial Sequence Synthetic peptide
CYXXXXSNVXCKXYRS

SEQ ID NO: 66 - protein, 16 aa, Artificial Sequence
  Description of Artificial Sequence Synthetic peptide
CYXXXXSQVXCKXYRS

SEQ ID NO: 67 - protein, 16 aa, Artificial Sequence
  Description of Artificial Sequence Synthetic peptide
CYXXXXSXVLCKXYRS

SEQ ID NO: 68 - protein, 16 aa, Artificial Sequence
  Description of Artificial Sequence Synthetic peptide
CYXXXXSXVICKXYRS

SEQ ID NO: 69 - protein, 16 aa, Artificial Sequence
  Description of Artificial Sequence Synthetic peptide
CYXXXXSXVXCKRYRS

SEQ ID NO: 70 - protein, 16 aa, Artificial Sequence
  Description of Artificial Sequence Synthetic peptide
CYXXXXSXVXCKKYRS

SEQ ID NO: 71 - protein, 16 aa, Artificial Sequence
  Description of Artificial Sequence Synthetic peptide
CYFDDNSNVICKKYRS

SEQ ID NO: 72 - protein, 16 aa, Artificial Sequence
  Description of Artificial Sequence Synthetic peptide
CYFDDNSNVLCKKYRS

SEQ ID NO: 73 - protein, 16 aa, Artificial Sequence
  Description of Artificial Sequence Synthetic peptide
CYFDDNSQVICKKYRS

SEQ ID NO: 74 - protein, 16 aa, Artificial Sequence
  Description of Artificial Sequence Synthetic peptide
CYFDDNSQVLCKKYRS

SEQ ID NO: 75 - protein, 16 aa, Artificial Sequence
  Description of Artificial Sequence Synthetic peptide
CYFDDSSNVICKRYRS

SEQ ID NO: 76 - protein, 16 aa, Artificial Sequence
  Description of Artificial Sequence Synthetic peptide
CYFDDSSNVLCKKYRS

SEQ ID NO: 77 - protein, 16 aa, Artificial Sequence
  Description of Artificial Sequence Synthetic peptide
CYFDDSSQVICKKYRS

SEQ ID NO: 78 - DNA, 20 bp, Artificial Sequence
  Description of Artificial Sequence Synthetic primer
cttgtgtctgcacgacctgt

SEQ ID NO: 79 - DNA, 19 bp, Artificial Sequence
  Description of Artificial Sequence Synthetic primer
gtggaatattgccggtgca

SEQ ID NO: 80 - DNA, 20 bp, Artificial Sequence
  Description of Artificial Sequence Synthetic primer
tgtaaactccctccacccca

SEQ ID NO: 81 - DNA, 19 bp, Artificial Sequence
  Description of Artificial Sequence Synthetic primer
atccgggagcttttccctg

SEQ ID NO: 82 - DNA, 19 bp, Artificial Sequence
  Description of Artificial Sequence Synthetic primer
tccacctgggtcatctcca

SEQ ID NO: 83 - DNA, 20 bp, Artificial Sequence
  Description of Artificial Sequence Synthetic primer
tggcctgactggttgtcaga

SEQ ID NO: 84 - DNA, 20 bp, Artificial Sequence
  Description of Artificial Sequence Synthetic primer
ggacatgcgtgaggttgtgt

SEQ ID NO: 85 - DNA, 20 bp, Artificial Sequence
  Description of Artificial Sequence Synthetic primer
gcggcctatgccatttacac

SEQ ID NO: 86 - DNA, 20 bp, Artificial Sequence
  Description of Artificial Sequence Synthetic primer
cctctgttcttgctgcgctc

SEQ ID NO: 87 - DNA, 19 bp, Artificial Sequence
  Description of Artificial Sequence Synthetic primer
gtagcgaggtggtggccat

SEQ ID NO: 88 - DNA, 21 bp, Artificial Sequence
  Description of Artificial Sequence Synthetic primer
ggacagctacgaaacgcaagt

SEQ ID NO: 89 - DNA, 20 bp, Artificial Sequence
  Description of Artificial Sequence Synthetic primer
tcgacgagtgtcagaatggc

SEQ ID NO: 90 - DNA, 20 bp, Artificial Sequence
  Description of Artificial Sequence Synthetic primer
tgccagtgacaatgccacat

SEQ ID NO: 91 - DNA, 19 bp, Artificial Sequence
  Description of Artificial Sequence Synthetic primer
agttccaacccatgcctcc

SEQ ID NO: 92 - DNA, 19 bp, Artificial Sequence
  Description of Artificial Sequence Synthetic primer
ttaaacctgtcccggcaca

SEQ ID NO: 93 - DNA, 20 bp, Artificial Sequence
  Description of Artificial Sequence Synthetic primer
tgaagcagaccatccacgag

SEQ ID NO: 94 - DNA, 19 bp, Artificial Sequence
  Description of Artificial Sequence Synthetic primer
agcgagatcaaagggctgg

SEQ ID NO: 95 - DNA, 20 bp, Artificial Sequence
  Description of Artificial Sequence Synthetic primer
aggagaatggcttctcacca

SEQ ID NO: 96 - DNA, 21 bp, Artificial Sequence
  Description of Artificial Sequence Synthetic primer
ccattgaagcatcttggttcg

SEQ ID NO: 97 - DNA, 20 bp, Artificial Sequence
  Description of Artificial Sequence Synthetic primer
tcgtctgtttccagggttgg

SEQ ID NO: 98 - DNA, 20 bp, Artificial Sequence
  Description of Artificial Sequence Synthetic primer
tgcaaggcaaccacactgac

SEQ ID NO: 99 - DNA, 20 bp, Artificial Sequence
  Description of Artificial Sequence Synthetic primer
ccctgtcactgccattgttg

SEQ ID NO: 100 - DNA, 19 bp, Artificial Sequence
  Description of Artificial Sequence Synthetic primer
ttccgtcaaagcagccact

SEQ ID NO: 101 - DNA, 20 bp, Artificial Sequence
  Description of Artificial Sequence Synthetic primer
cgctgttccagcggttagac

SEQ ID NO: 102 - DNA, 19 bp, Artificial Sequence
  Description of Artificial Sequence Synthetic primer
agtctcgatgggcgattgc

SEQ ID NO: 103 - DNA, 20 bp, Artificial Sequence
  Description of Artificial Sequence Synthetic primer
aagctggagtgcacctcgtt

SEQ ID NO: 104 - DNA, 19 bp, Artificial Sequence
  Description of Artificial Sequence Synthetic primer
caggacagtgcgctggttc

SEQ ID NO: 105 - DNA, 20 bp, Artificial Sequence
  Description of Artificial Sequence Synthetic primer
catcttgctggcagggtaca

SEQ ID NO: 106 - DNA, 19 bp, Artificial Sequence
  Description of Artificial Sequence Synthetic primer
tgcctgcagtgttgatgca

SEQ ID NO: 107 - DNA, 20 bp, Artificial Sequence
  Description of Artificial Sequence Synthetic primer
ccagaagagcaggcagcttc

SEQ ID NO: 108 - DNA, 20 bp, Artificial Sequence
  Description of Artificial Sequence Synthetic primer
ggcacttcacaggcacacat

SEQ ID NO: 109 - DNA, 19 bp, Artificial Sequence
  Description of Artificial Sequence Synthetic primer
ctgcctccattcctggctt

SEQ ID NO: 110 - DNA, 20 bp, Artificial Sequence
  Description of Artificial Sequence Synthetic primer
ggccataacagaagcggttg

SEQ ID NO: 111 - DNA, 19 bp, Artificial Sequence
  Description of Artificial Sequence Synthetic primer
ctcaggcgctgtttcttgc